SEQUENCE LISTING (146 sequences)

SEQ ID NO: 1 - DNA, 388 bp, Artificial Sequence
  QUEL-0101 VH domain nucleotide
caggttcaactggtggagtctgggggaggcgtggtccagcctgggaggtccctgagactctcctgtgcagcctctggattcgccttcaataactatggcatgcactgggtccgccagggtccaggcagggggctggaatgggtggctattatatcatatgatggaacaactcaatactctgtagactccgtgaagggccgattcaccatctccagagacaattccaagaacacgttgtatctgcaaatgaacagcctgagaactgaggacacggctctatattactgtgcgaaagaggcgggggatttctatggttcggggaattatgggtaccacaagtatggtttggacgtctggggccaagggaccacggtcaccgtctcctcag

SEQ ID NO: 2 - protein, 129 aa, Artificial Sequence
  QUEL-0101 VH domain amino acid
QVQLVESGGGVVQPGRSLRLSCAASGFAFNNYGMHWVRQGPGRGLEWVAIISYDGTTQYSVDSVKGRFTISRDNSKNTLYLQMNSLRTEDTALYYCAKEAGDFYGSGNYGYHKYGLDVWGQGTTVTVSS

SEQ ID NO: 3 - protein, 8 aa, Artificial Sequence
  QUEL-0101 HCDR1
GFAFNNYG

SEQ ID NO: 4 - protein, 8 aa, Artificial Sequence
  QUEL-0101 HCDR2
ISYDGTTQ

SEQ ID NO: 5 - protein, 22 aa, Artificial Sequence
  QUEL-0101 HCDR3, QUEL-0103 HCDR3
AKEAGDFYGSGNYGYHKYGLDV

SEQ ID NO: 6 - protein, 322 aa, Artificial Sequence
  QUEL-0101 VL domain nucleotide
GACATCCAGATGACCCAGTCTCCATCCTCCCTGTCTGCATCTGTAGGAGACAGAGTCACCATCACTTGCCGGGCGAGTCAGGACATTAGCAATTATTTAGCCTGGTATCAGCAGAAGCCAGGGAAAATTCCTAAGCTCCTGATCTATGCTGCATCCACTTTGCAATCAGGGGTCCCATCTCGGTTCAGTGGCAGTGGATCTGGGACAGATTTCACTCTCACCATCAGCAGCCTGCAGCCTGAAGATGTTGCAAGTTATTACTGTCAAAAATATAGCAGTGCCCCGCTCACTTTCGGCGGAGGGACCAAGGTGGAGATCAAAC

SEQ ID NO: 7 - protein, 107 aa, Artificial Sequence
  QUEL-0101 VL domain amino acid
DIQMTQSPSSLSASVGDRVTITCRASQDISNYLAWYQQKPGKIPKLLIYAASTLQSGVPSRFSGSGSGTDFTLTISSLQPEDVASYYCQKYSSAPLTFGGGTKVEIK

SEQ ID NO: 8 - protein, 6 aa, Artificial Sequence
  QUEL-0101 LCDR1, QUEL-0102 LCDR1, QUEL-0103 LCDR1, QUEL-0104 LCDR1, QUEL-0105 LCDR1
QDISNY

SEQ ID NO: 9 - skipped

SEQ ID NO: 10 - protein, 9 aa, Artificial Sequence
  QUEL-0101 LCDR3
QKYSSAPLT

SEQ ID NO: 11 - DNA, 373 bp, Artificial Sequence
  QUEL-0201 VH domain nucleotide
caggtccagcttgtgcagtctggggctgaggtgaaggagcctggggcctcagtgagggtttcctgtaaggcttctggatacaccttcattagtcacgctatacattgggtgcgccaggcccccggacaaagacttgagtggatgggatggatcaacgctgtcaatggaaacacaaaatattcacagaagttccaggacagagtcacctttaccagggacacatccgcgagcacagcctacatggacctgaacagcctgagagctgaagacacggctctttattactgtgcgagagagggatacacctatggtgtccactactcgtacggtatggacgtctggggccaagggaccacggtcaccgtctcgtcag

SEQ ID NO: 12 - protein, 124 aa, Artificial Sequence
  QUEL-0201 VH domain amino acid
QVQLVQSGAEVKEPGASVRVSCKASGYTFISHAIHWVRQAPGQRLEWMGWINAVNGNTKYSQKFQDRVTFTRDTSASTAYMDLNSLRAEDTALYYCAREGYTYGVHYSYGMDVWGQGTTVTVSS

SEQ ID NO: 13 - protein, 8 aa, Artificial Sequence
  QUEL-0201 HCDR1
GYTFISHA

SEQ ID NO: 14 - protein, 8 aa, Artificial Sequence
  QUEL-0201 HCDR2
INAVNGNT

SEQ ID NO: 15 - protein, 17 aa, Artificial Sequence
  QUEL-0201 HCDR3
AREGYTYGVHYSYGMDV

SEQ ID NO: 16 - DNA, 337 bp, Artificial Sequence
  QUEL-0201 VL domain nucleotide
cagtctgtgctgacgcagccgccctcagtgtctggggccccagggcagagggtcaccatctcctgcactgggagcagttccaatatcggggcaggttatgatgtacactggtaccagcagcttccaggaacagtcctcaaactcctcatttatggtaacaacaatcggccctcaggggtccctgaccgattctctggctccaactctggcacctcagcctccctggccatcactgggctccagcctgaggatgaggctgattattactgccagtcctatgacagcagcctgagtggttattgggtgttcggcggagggaccaagctgaccgtcctag

SEQ ID NO: 17 - protein, 112 aa, Artificial Sequence
  QUEL-0201 VL domain amino acid
QSVLTQPPSVSGAPGQRVTISCTGSSSNIGAGYDVHWYQQLPGTVLKLLIYGNNNRPSGVPDRFSGSNSGTSASLAITGLQPEDEADYYCQSYDSSLSGYWVFGGGTKLTVL

SEQ ID NO: 18 - protein, 9 aa, Artificial Sequence
  QUEL-0201 LCDR1
SSNIGAGYD

SEQ ID NO: 19 - skipped

SEQ ID NO: 20 - protein, 12 aa, Artificial Sequence
  QUEL-0201 LCDR3
QSYDSSLSGYWV

SEQ ID NO: 21 - DNA, 355 bp, Artificial Sequence
  QUEL-0301 VH domain nucleotide
gaggtgcaactggtggagtctgggggaggcttggtccagcctggggggtccctgagactctcctgcgcagcttctggattcacctttagtaactcttggatgaactgggtccgccagactccagggaaggggctggagtgggtggccaatatagaccaagatggagatcagaaatactatgtggactctgtgaagggccgattcaccatctccagagacaacgccaagaactcactgtatctgcaaatgaacagcctgagagccgaggacacggctgtctattactgtgcgagagaaataactggaactacatttgactactggggccagggaaccctggtcaccgtctcctcag

SEQ ID NO: 22 - protein, 118 aa, Artificial Sequence
  QUEL-0301 VH domain amino acid
EVQLVESGGGLVQPGGSLRLSCAASGFTFSNSWMNWVRQTPGKGLEWVANIDQDGDQKYYVDSVKGRFTISRDNAKNSLYLQMNSLRAEDTAVYYCAREITGTTFDYWGQGTLVTVSS

SEQ ID NO: 23 - protein, 8 aa, Artificial Sequence
  QUEL-0301 HCDR1, QUEL-0302 HCDR1
GFTFSNSW

SEQ ID NO: 24 - protein, 8 aa, Artificial Sequence
  QUEL-0301 HCDR2
IDQDGDQK

SEQ ID NO: 25 - protein, 11 aa, Artificial Sequence
  QUEL-0301 HCDR3
AREITGTTFDY

SEQ ID NO: 26 - DNA, 331 bp, Artificial Sequence
  QUEL-0301 VL domain nucleotide
cagtctgtgctgactcagccaccctcagcgtctgggacccccgggcagagggtcaccatctcttgttctggaagcagctccaacatcggaagtaatattgtaaactggtaccagcaactcccaggaacggcccccaaactcctcatctctagtaataatcagcggccctcaggggtccctgaccgattctctggctccaagtctggcacctcagcctccctggccatcagtgggctccagtctgaggatgaggctgattattactgtacaacatgggatgacagcctggatggtccggtgttcggcggagggaccaagctgaccgtcctgg

SEQ ID NO: 27 - protein, 110 aa, Artificial Sequence
  QUEL-0301 VL domain amino acid
QSVLTQPPSASGTPGQRVTISCSGSSSNIGSNIVNWYQQLPGTAPKLLISSNNQRPSGVPDRFSGSKSGTSASLAISGLQSEDEADYYCTTWDDSLDGPVFGGGTKLTVL

SEQ ID NO: 28 - protein, 8 aa, Artificial Sequence
  QUEL-0301 LCDR1
SSNIGSNI

SEQ ID NO: 29 - skipped

SEQ ID NO: 30 - protein, 11 aa, Artificial Sequence
  QUEL-0301 LCDR3
TTWDDSLDGPV

SEQ ID NO: 31 - protein, 394 aa, Homo sapiens
  MISC_FEATURE (1)..(394): human GFRAL
MIVFIFLAMGLSLENEYTSQTNNCTYLREQCLRDANGCKHAWRVMEDACNDSDPGDPCKMRNSSYCNLSIQYLVESNFQFKECLCTDDFYCTVNKLLGKKCINKSDNVKEDKFKWNLTTRSHHGFKGMWSCLEVAEACVGDVVCNAQLASYLKACSANGNPCDLKQCQAAIRFFYQNIPFNIAQMLAFCDCAQSDIPCQQSKEALHSKTCAVNMVPPPTCLSVIRSCQNDELCRRHYRTFQSKCWQRVTRKCHEDENCISTLSKQDLTCSGSDDCKAAYIDILGTVLQVQCTCRTITQSEESLCKIFQHMLHRKSCFNYPTLSNVKGMALYTRKHANKITLTGFHSPFNGEVIYAAMCMTVTCGILLLVMVKLRTSRISSKARDPSPIQIPGEL

SEQ ID NO: 32 - protein, 393 aa, Mus musculus
  MISC_FEATURE (1)..(393): mouse GFRAL
MLVFIFLAVTLSSENESSSQTNDCAHLIQKCLIDANGCEQSWRSMEDTCLTPGDSCKINNSLHCNLSIQALVEKNFQFKECLCMDDLHCTVNKLFGKKCTNKTDNMEKDNKDKWNLTTTPFYHGFKQMQSCLEVTEACVGDVVCNAQLALYLKACSANGNLCDVKHCQAAIRFFYQNMPFNTAQMLAFCDCAQSDIPCQQSKETLHSKPCALNIVPPPTCLSVIHTCRNDELCRTHYRTFQTECWPHITGKCHEDETCISMLGKQDLTCSGSESCRAAFLGTFGTVLQVPCACRGVTQAEEHVCMIFQHMLHSKSCFNYPTPNVKDISSYEKKNSKEITLTGFNSFFNGELLYVVVCMAVTCGILFLVMLKLRIQSEKRDPSSIEIAGGVIIQ

SEQ ID NO: 33 - DNA, 990 bp, Homo sapiens
  misc_feature (1)..(990): Human Heavy Chain Constant Region (IGHG1*01) Nucleotide Sequence
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 34 - protein, 330 aa, Homo sapiens [sequence omitted]

SEQ ID NO: 35 - DNA, 990 bp, Homo sapiens
  misc_feature (1)..(990): Human Heavy Chain Constant Region (IGHG1*02 or IGHG1*05) Nucleotide Sequence
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 36 - protein, 330 aa, Homo sapiens [sequence omitted]

SEQ ID NO: 37 - DNA, 990 bp, Homo sapiens
  misc_feature (1)..(990): Human Heavy Chain Constant Region (IGHG1*03) Nucleotide Sequence (Y14737)
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 38 - protein, 330 aa, Homo sapiens
  MISC_FEATURE (1)..(330): Human Heavy Chain Constant Region (IGHG1*03) Protein Sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - DNA, 990 bp, Homo sapiens
  misc_feature: Human Heavy Chain Constant Region (IGHG1*04) Nucleotide Sequence
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacatcttctcatgctccgtgatgcatgaggctctgcacaaccactacacgcagaagagcctctccctgtctccgggtaaa

SEQ ID NO: 40 - protein, 330 aa, Homo sapiens
  MISC_FEATURE (1)..(330): Human Heavy Chain Constant Region (IGHG1*04) Protein Sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNIFSCSVMHEALHNHYTQKSLSLSPGK

SEQ ID NO: 41 - DNA, 990 bp, Artificial Sequence
  Disabled Human IGHG1*01 Heavy Chain Constant Region Nucleotide Sequence.
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagtggagcccaaatcttgtgacaaaactcacacatgcccaccgtgcccagcacctgaactcgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 42 - protein, 330 aa, Artificial Sequence
  Disabled Human IGHG1*01 Heavy Chain Constant Region Amino Acid Sequence.
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - DNA, 978 bp, Homo sapiens
  misc_feature (1)..(978): Human Heavy Chain Constant Region (IGHG2*01 or IGHG2*03 or IGHG2*05) Nucleotide Sequence
gcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 44 - protein, 326 aa, Homo sapiens
  MISC_FEATURE (1)..(326): Human Heavy Chain Constant Region (IGHG2*01) Protein Sequence
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 45 - DNA, 978 bp, Homo sapiens
  misc_feature (1)..(978): Human Heavy Chain Constant Region (IGHG2*02) Nucleotide Sequence
gcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccggctgtcctacagtcctcaggactctactccctcagcagcgtggtgaccgtgacctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcatggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 46 - protein, 326 aa, Homo sapiens
  MISC_FEATURE (1)..(326): Human Heavy Chain Constant Region (IGHG2*02) Protein Sequence
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 47 - DNA, 978 bp, Homo sapiens
  misc_feature (1)..(978): Human Heavy Chain Constant Region (IGHG2*04) Nucleotide Sequence
gcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcagcttgggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 48 - protein, 326 aa, Homo sapiens
  MISC_FEATURE (1)..(326): Human Heavy Chain Constant Region (IGHG2*04) Protein Sequence
ASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 49 - DNA, 978 bp, Homo sapiens
  misc_feature (1)..(978): Human Heavy Chain Constant Region (IGHG2*06) Nucleotide Sequence
gcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccggctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatctccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 50 - protein, 326 aa, Homo sapiens
  MISC_FEATURE (1)..(326): Human Heavy Chain Constant Region (IGHG2*06) Protein Sequence
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 51 - DNA, 981 bp, Homo sapiens
  misc_feature (1)..(981): Human Heavy Chain Constant Region (IGHG4*01 or IGHG4*04) Nucleotide Sequence
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 52 - protein, 327 aa, Homo sapiens
  MISC_FEATURE (1)..(327): Human Heavy Chain Constant Region (IGHG4*01) Protein Sequence (P01861)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 53 - DNA, 981 bp, Homo sapiens
  misc_feature (1)..(981): Human Heavy Chain Constant Region (IGHG4*02) Nucleotide Sequence
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccgtgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcgtgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacgcagaagagcctctccctgtctctgggtaaa

SEQ ID NO: 54 - protein, 327 aa, Homo sapiens
  MISC_FEATURE (1)..(327): Human Heavy Chain Constant Region (IGHG4*02) Protein Sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 55 - DNA, 981 bp, Homo sapiens
  misc_feature (1)..(981): Human Heavy Chain Constant Region (IGHG4*03) Nucleotide Sequence
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaagctcaccgtggacaagagcaggtggcaggaggggaacgtcttctcatgctccgtgatgcatgaggctctgcacaaccactacacgcagaagagcctctccctgtctctgggtaaa

SEQ ID NO: 56 - protein, 327 aa, Homo sapiens
  MISC_FEATURE (1)..(327): Human Heavy Chain Constant Region (IGHG4*03) Protein Sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 57 - DNA, 981 bp, Artificial Sequence
  Human Heavy Chain Constant Region (IGHG4-PE) Nucleotide Sequence Version A
gcctccaccaagggcccatccgtcttccccctggcgccctgctccaggagcacctccgagagcacggccgccctgggctgcctggtcaaggactacttccccgaaccagtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcgcctgaatttgaggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcatcgatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggatccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 58 - DNA, 981 bp, Artificial Sequence
  Human Heavy Chain Constant Region (IGHG4-PE) Nucleotide Sequence Version B
gcctccaccaagggacctagcgtgttccctctcgccccctgttccaggtccacaagcgagtccaccgctgccctcggctgtctggtgaaagactactttcccgagcccgtgaccgtctcctggaatagcggagccctgacctccggcgtgcacacatttcccgccgtgctgcagagcagcggactgtatagcctgagcagcgtggtgaccgtgcccagctccagcctcggcaccaaaacctacacctgcaacgtggaccacaagccctccaacaccaaggtggacaagcgggtggagagcaagtacggccccccttgccctccttgtcctgcccctgagttcgagggaggaccctccgtgttcctgtttccccccaaacccaaggacaccctgatgatctcccggacacccgaggtgacctgtgtggtcgtggacgtcagccaggaggaccccgaggtgcagttcaactggtatgtggacggcgtggaggtgcacaatgccaaaaccaagcccagggaggagcagttcaattccacctacagggtggtgagcgtgctgaccgtcctgcatcaggattggctgaacggcaaggagtacaagtgcaaggtgtccaacaagggactgcccagctccatcgagaagaccatcagcaaggctaagggccagccgagggagccccaggtgtataccctgcctcctagccaggaagagatgaccaagaaccaagtgtccctgacctgcctggtgaagggattctacccctccgacatcgccgtggagtgggagagcaatggccagcccgagaacaactacaaaacaacccctcccgtgctcgatagcgacggcagcttctttctctacagccggctgacagtggacaagagcaggtggcaggagggcaacgtgttctcctgttccgtgatgcacgaggccctgcacaatcactacacccagaagagcctctccctgtccctgggcaag

SEQ ID NO: 59 - DNA, 981 bp, Artificial Sequence
  Human Heavy Chain Constant Region (IGHG4-PE) Nucleotide Sequence Version C
gccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 60 - protein, 327 aa, Artificial Sequence
  Human Heavy Chain Constant Region (IGHG4-PE) Protein Sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 61 - DNA, 981 bp, Artificial Sequence
  Inactivated Human Heavy Chain Constant Region (IGHG4) Nucleotide Sequence
gcctccaccaagggcccatccgtcttccccctggcgccctgctccaggagcacctccgagagcacggccgccctgggctgcctggtcaaggactacttccccgaaccagtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcgcctccagttgcggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcatcgatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggatccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 62 - protein, 327 aa, Artificial Sequence
  Inactivated Human Heavy Chain Constant Region (IGHG4) Protein Sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 63 - DNA, 321 bp, Homo sapiens
  misc_feature (1)..(321): Human Ck Light Chain Constant Region (IGKC*01) Nucleotide Sequence
cgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 64 - protein, 107 aa, Homo sapiens [sequence omitted]

SEQ ID NO: 65 - DNA, 321 bp, Homo sapiens
  misc_feature (1)..(321): Ck Light Chain Constant Region (IGKC*02) Nucleotide Sequence
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggagagcaaggacagcacctacagcctcagcagcaccctgacgctgagcaaagcagactacgagaaacacaaagtctacgccggcgaagtcacccatcagggcctgagctcgcccgtcacaaagagcttcaacaggggagagtgt

SEQ ID NO: 66 - protein, 107 aa, Homo sapiens
  MISC_FEATURE (1)..(107): Ck Light Chain Constant Region (IGKC*02) Amino Acid Sequence
RTVAAPSVFIFPPSDEQLKSGTASVVCLLNNFYPREAKVQWKVDNALQSGNSQESVTEQESKDSTYSLSSTLTLSKADYEKHKVYAGEVTHQGLSSPVTKSFNRGEC

SEQ ID NO: 67 - DNA, 321 bp, Homo sapiens
  misc_feature (1)..(321): Ck Light Chain Constant Region (IGKC*03) Nucleotide Sequence
cgaactgtggctgcaccatctgtcttcatcttcccgccatctgatgagcagttgaaatctggaactgcctctgttgtgtgcctgctgaataacttctatcccagagaggccaaagtacagcggaaggtggataacgccctccaatcgggtaactcccaggagagtgtcacagagcaggagagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 68 - protein, 107 aa, Homo sapiens
  MISC_FEATURE (1)..(107): Ck Light Chain Constant Region (IGKC*03) Amino Acid Sequence
RTVAAPSVFIFPPSDEQLKSGTASVVCLLNNFYPREAKVQRKVDNALQSGNSQESVTEQESKDSTYSLSSTLTLSKADYEKHKVYACEVTHQGLSSPVTKSFNRGEC

SEQ ID NO: 69 - DNA, 321 bp, Homo sapiens
  misc_feature (1)..(321): Ck Light Chain Constant Region (IGKC*04) Nucleotide Sequence
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgt

SEQ ID NO: 70 - protein, 107 aa, Homo sapiens
  MISC_FEATURE (1)..(107): Ck Light Chain Constant Region (IGKC*04) Amino Acid Sequence
RTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 71 - DNA, 321 bp, Homo sapiens
  misc_feature (1)..(321): Ck Light Chain Constant Region (IGKC*05) Nucleotide Sequence
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcaacaccctgacgctgagcaaagcagactacgagaaacacaaagtctacgcctgcgaagtcacccatcagggcctgagctcgcccgtcacaaagagcttcaacaggggagagtgc

SEQ ID NO: 72 - protein, 107 aa, Homo sapiens
  MISC_FEATURE (1)..(107): Ck Light Chain Constant Region (IGKC*05) Amino Acid Sequence
RTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 73 - DNA, 312 bp, Homo sapiens
  misc_feature (1)..(312): Cl Light Chain Constant Region (IGLC1*01) Nucleotide Sequence (ENST00000390321.2
cccaaggccaaccccacggtcactctgttcccgccctcctctgaggagctccaagccaacaaggccacactagtgtgtctgatcagtgacttctacccgggagctgtgacagtggcttggaaggcagatggcagccccgtcaaggcgggagtggagacgaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 74 - protein, 104 aa, Homo sapiens
  MISC_FEATURE (1)..(104): Cl Light Chain Constant Region (IGLC1*01) Amino Acid Sequence (A0A075B6K8)
PKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 75 - DNA, 318 bp, Homo sapiens
  misc_feature (1)..(318): Cl Light Chain Constant Region (IGLC1*02) Nucleotide Sequence Version A
ggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 76 - DNA, 318 bp, Homo sapiens; identical to SEQ ID NO: 75
  misc_feature (1)..(318): CL Light Chain Constant Region (IGLC1*02) Nucleotide Sequence Version B

SEQ ID NO: 77 - protein, 106 aa, Homo sapiens
  MISC_FEATURE (1)..(106): Cl Light Chain Constant Region (IGLC1*02) Amino Acid Sequence
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 78 - DNA, 318 bp, Homo sapiens
  misc_feature (1)..(318): Cl Light Chain Constant Region (IGLC2*01) Nucleotide Sequence Version A
ggccagcctaaggccgctccttctgtgaccctgttccccccatcctccgaggaactgcaggctaacaaggccaccctcgtgtgcctgatcagcgacttctaccctggcgccgtgaccgtggcctggaaggctgatagctctcctgtgaaggccggcgtggaaaccaccaccccttccaagcagtccaacaacaaatacgccgcctcctcctacctgtccctgacccctgagcagtggaagtcccaccggtcctacagctgccaagtgacccacgagggctccaccgtggaaaagaccgtggctcctaccgagtgctcc

SEQ ID NO: 79 - DNA, 318 bp, Homo sapiens
  misc_feature (1)..(318): Cl Light Chain Constant Region (IGLC2*01) Nucleotide Sequence Version B
ggccagcctaaagctgcccccagcgtcaccctgtttcctccctccagcgaggagctccaggccaacaaggccaccctcgtgtgcctgatctccgacttctatcccggcgctgtgaccgtggcttggaaagccgactccagccctgtcaaagccggcgtggagaccaccacaccctccaagcagtccaacaacaagtacgccgcctccagctatctctccctgacccctgagcagtggaagtcccaccggtcctactcctgtcaggtgacccacgagggctccaccgtggaaaagaccgtcgcccccaccgagtgctcc

SEQ ID NO: 80 - protein, 106 aa, Homo sapiens
  MISC_FEATURE: Cl Light Chain Constant Region (IGLC1*02) Amino Acid Sequence
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 81 - DNA, 318 bp, Homo sapiens
  misc_feature (1)..(318): Cl Light Chain Constant Region (IGLC2*02 or IGLC2*03) Nucleotide Sequence
ggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 82 - protein, 106 aa, Homo sapiens; identical to SEQ ID NO: 80
  MISC_FEATURE (1)..(106): Cl Light Chain Constant Region (IGLC2*02) Amino Acid Sequence

SEQ ID NO: 83 - DNA, 312 bp, Homo sapiens
  misc_feature (1)..(312): Cl Light Chain Constant Region (IGLC3*01) Nucleotide Sequence
cccaaggctgccccctcggtcactctgttcccaccctcctctgaggagcttcaagccaacaaggccacactggtgtgtctcataagtgacttctacccgggagccgtgacagttgcctggaaggcagatagcagccccgtcaaggcgggggtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagttgcccctacggaatgttca

SEQ ID NO: 84 - protein, 104 aa, Homo sapiens
  MISC_FEATURE (1)..(104): Cl Light Chain Constant Region (IGLC3*01) Amino Acid Sequence
PKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 85 - DNA, 318 bp, Homo sapiens
  misc_feature (1)..(318): Cl Light Chain Constant Region (IGLC3*02) Nucleotide Sequence
ggtcagcccaaggctgccccctcggtcactctgttcccaccctcctctgaggagcttcaagccaacaaggccacactggtgtgtctcataagtgacttctacccggggccagtgacagttgcctggaaggcagatagcagccccgtcaaggcgggggtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacggaatgttca

SEQ ID NO: 86 - protein, 106 aa, Homo sapiens
  MISC_FEATURE (1)..(106): Cl Light Chain Constant Region (IGLC1*02) Amino Acid Sequence
GQPKAAPSVTLFPPSSEELQANKATLVCLISDFYPGPVTVAWKADSSPVKAGVETTTPSKQSNNKYAASSYLSLTPEQWKSHKSYSCQVTHEGSTVEKTVAPTECS

SEQ ID NO: 87 - DNA, 318 bp, Homo sapiens
  misc_feature (1)..(318): Cl Light Chain Constant Region (IGLC3*03) Nucleotide Sequence
ggtcagcccaaggctgccccctcggtcactctgttcccaccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacagaatgttca

SEQ ID NO: 88 - protein, 106 aa, Homo sapiens
  MISC_FEATURE (1)..(106): Cl Light Chain Constant Region (IGLC3*03) Amino Acid Sequence
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 89 - DNA, 318 bp, Homo sapiens
  misc_feature (1)..(318): Cl Light Chain Constant Region (IGLC3*04) Nucleotide Sequence
ggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttca

SEQ ID NO: 90 - protein, 106 aa, Homo sapiens; identical to SEQ ID NO: 80
  MISC_FEATURE (1)..(106): Cl Light Chain Constant Region (IGLC3*04) Amino Acid Sequence

SEQ ID NO: 91 - DNA, 318 bp, Homo sapiens
  misc_feature (1)..(318): Cl Light Chain Constant Region (IGLC6*01) Nucleotide Sequence
ggtcagcccaaggctgccccatcggtcactctgttcccgccctcctctgaggagcttcaagccaacaaggccacactggtgtgcctgatcagtgacttctacccgggagctgtgaaagtggcctggaaggcagatggcagccccgtcaacacgggagtggagaccaccacaccctccaaacagagcaacaacaagtacgcggccagcagctacctgagcctgacgcctgagcagtggaagtcccacagaagctacagctgccaggtcacgcatgaagggagcaccgtggagaagacagtggcccctgcagaatgttca

SEQ ID NO: 92 - protein, 106 aa, Homo sapiens
  MISC_FEATURE (1)..(106): Cl Light Chain Constant Region (IGLC6*01) Amino Acid Sequence
GQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECS

SEQ ID NO: 93 - DNA, 318 bp, Homo sapiens
  misc_feature (1)..(318): Cl Light Chain Constant Region (IGLC7*01 or IGLC7*02) Nucleotide Sequence
ggtcagcccaaggctgccccatcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 94 - protein, 106 aa, Homo sapiens
  MISC_FEATURE (1)..(106): Cl Light Chain Constant Region (IGLC7*01) Amino Acid Sequence
GQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 95 - DNA, 318 bp, Homo sapiens
  misc_feature (1)..(318): Cl Light Chain Constant Region (IGLC7*03) Nucleotide Sequence
ggtcagcccaaggctgccccctcggtcactctgttcccaccctcctctgaggagcttcaagccaacaaggccacactggtgtgtctcgtaagtgacttcaacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 96 - protein, 106 aa, Homo sapiens
  MISC_FEATURE (1)..(106): Cl Light Chain Constant Region (IGLC7*03) Amino Acid Sequence
GQPKAAPSVTLFPPSSEELQANKATLVCLVSDFNPGAVTVAWKADGSPVKVGVETTKPSKQSNNKYAASSYLSLTPEQWKSHRSYSCRVTHEGSTVEKTVAPAECS

SEQ ID NO: 97 - DNA, 388 bp, Artificial Sequence
  QUEL-0102 VH domain nucleotide
caggttcaactggtggagtctgggggaggcgtggtccagcctgggaggtccctgagactctcctgtgcagcctctggattcaccctcaaaaactatggcatgcactgggtccgccagggtccaggcaaggggctggagtgggtggcaattatatcatatgatggaacatttaaatattctgtagactccgtgaagggccgattcaccatctccagagacaattccaagaacacgttgtatctgcaaatgaacagcctgagaactgaggacacggctctgtattactgtgcgaaagaggcgggggatttctatggttcggggagttatgggtaccacaagtatggtttggacgtctggggccaagggaccacggtcaccgtctcctcag

SEQ ID NO: 98 - protein, 129 aa, Artificial Sequence
  QUEL-0102 VH domain amino acid
QVQLVESGGGVVQPGRSLRLSCAASGFTLKNYGMHWVRQGPGKGLEWVAIISYDGTFKYSVDSVKGRFTISRDNSKNTLYLQMNSLRTEDTALYYCAKEAGDFYGSGSYGYHKYGLDVWGQGTTVTVSS

SEQ ID NO: 99 - protein, 8 aa, Artificial Sequence
  QUEL-0102 HCDR1
GFTLKNYG

SEQ ID NO: 100 - protein, 8 aa, Artificial Sequence
  QUEL-0102 HCDR2
ISYDGTFK

SEQ ID NO: 101 - protein, 22 aa, Artificial Sequence
  QUEL-0102 HCDR3
AKEAGDFYGSGSYGYHKYGLDV

SEQ ID NO: 102 - DNA, 322 bp, Artificial Sequence
  QUEL-0102 VL domain nucleotide
gacgtccagatgacccagtctccatcctccctgtctgcatctgtaggagacagagtcaccatcacttgccgggcgagtcaggacattagcaattatttagcctggtatcagcagcaaccagggaaagttcctaaactcctgatctatgctgcatccactttgcaatcaggggtcccatctcggttcagtggcagtggatctgggacagatttcactctcaccatcagcagcctgcagcctgaagatgttgcaagttattactgtcaaaaatataacagtgccccgctcactttcggcggagggaccgaggtggagatcaagc

SEQ ID NO: 103 - protein, 107 aa, Artificial Sequence
  QUEL-0102 VL domain amino acid
DVQMTQSPSSLSASVGDRVTITCRASQDISNYLAWYQQQPGKVPKLLIYAASTLQSGVPSRFSGSGSGTDFTLTISSLQPEDVASYYCQKYNSAPLTFGGGTEVEIK

SEQ ID NO: 104 - protein, 9 aa, Artificial Sequence
  QUEL-0102 LCDR3, QUEL-0103 LCDR3, QUEL-0104 LCDR3, QUEL-0105 LCDR3
QKYNSAPLT

SEQ ID NO: 105 - DNA, 388 bp, Artificial Sequence
  QUEL-0103 VH domain nucleotide
caggttcaactggtggagtctgggggaggcgtggtccagcctgggaggtccctgagactctcctgtgcagcctctggattcaccttcaataactatggcatgcactgggtccgccagggtccaggcaaggggctggagtgggtggctattatatcatatgatggaacaactaaatactctgtagactccgtgaagggccgattcaccatctccagagacaattccaagaacacgttgtatctacaaatgaacagcctgagaactgaggacacggctctgtatttctgtgcgaaagaggcgggggatttctatggttcggggaattatgggtaccacaagtatggtttggacgtctggggccaagggaccacggtcaccgtctcctcag

SEQ ID NO: 106 - protein, 129 aa, Artificial Sequence
  QUEL-0103 VH domain amino acid
QVQLVESGGGVVQPGRSLRLSCAASGFTFNNYGMHWVRQGPGKGLEWVAIISYDGTTKYSVDSVKGRFTISRDNSKNTLYLQMNSLRTEDTALYFCAKEAGDFYGSGNYGYHKYGLDVWGQGTTVTVSS

SEQ ID NO: 107 - protein, 8 aa, Artificial Sequence
  QUEL-0103 HCDR1, QUEL-0104 HCDR1
GFTFNNYG

SEQ ID NO: 108 - protein, 8 aa, Artificial Sequence
  QUEL-0103 HCDR2, QUEL-0104 HCDR2, QUEL-0105 HCDR2
ISYDGTTK

SEQ ID NO: 109 - DNA, 322 bp, Artificial Sequence
  QUEL-0103 VL domain nucleotide
gacatccagatgacccagtctccatcctccctgtctgcatctgtaggagacagagtcaccatcacttgccgggcgagtcaggacattagcaattatttagcctggtatcagcagaaaccagggaaagttcctaagctcctgatctatgctgcatccactttgcaatcaggggtcccatctcggttcagtggcagtggatctgggacagatttcactctcaccatcagcagcctgcagcctgaagatgttgcaagttattactgtcaaaaatataacagtgccccgctcactttcggcggagggaccgaggtggagatcaagc

SEQ ID NO: 110 - protein, 107 aa, Artificial Sequence
  QUEL-0103 VL domain amino acid
DIQMTQSPSSLSASVGDRVTITCRASQDISNYLAWYQQKPGKVPKLLIYAASTLQSGVPSRFSGSGSGTDFTLTISSLQPEDVASYYCQKYNSAPLTFGGGTEVEIK

SEQ ID NO: 111 - DNA, 388 bp, Artificial Sequence
  QUEL-0104 VH domain nucleotide
caggttcaactggtggagtctgggggaggcgtggtccagcctgggaggtccctgagactctcctgtgcagcctctggattcaccttcaataactatggcatgcactgggtccgccagggtccaggcaaggggctggagtgggtggctattatatcatatgatggaacaactaaatactctgtagactccgtgaagggccgattcactatctccagagacaattccaagaacacgttgtatatgcaaatggacagcctgagaacggaggacacggctctgtattactgtgcgaaggaggcgggggatttctatggttcggggaattatgggtacctctactatggtttggacgtctggggccaagggaccacggtcaccgtctcctcag

SEQ ID NO: 112 - protein, 129 aa, Artificial Sequence
  QUEL-0104 VH domain amino acid
QVQLVESGGGVVQPGRSLRLSCAASGFTFNNYGMHWVRQGPGKGLEWVAIISYDGTTKYSVDSVKGRFTISRDNSKNTLYMQMDSLRTEDTALYYCAKEAGDFYGSGNYGYLYYGLDVWGQGTTVTVSS

SEQ ID NO: 113 - protein, 22 aa, Artificial Sequence
  QUEL-0104 HCDR3
AKEAGDFYGSGNYGYLYYGLDV

SEQ ID NO: 114 - DNA, 322 bp, Artificial Sequence
  QUEL-0104 VL domain nucleotide
gacatccagatgacccagtctccatcctccctgtctgcatctgtaggagacagagtcaccatcacttgccgggcgagtcaggacattagcaattatttagcctggtatcagcagaaaccagggaaagttcctaacctcctgatctttgctgcatccactttgcaatcaggggtcccatctcggttcagtggcagtggatctgggacagatttcactctcaccatcagcagcctgcagcctgaagatgttgcaacttattactgtcaaaagtataatagtgccccgctcactttcggcggagggaccaaggtggagatcaacc

SEQ ID NO: 115 - protein, 107 aa, Artificial Sequence
  QUEL-0104 VL domain amino acid
DIQMTQSPSSLSASVGDRVTITCRASQDISNYLAWYQQKPGKVPNLLIFAASTLQSGVPSRFSGSGSGTDFTLTISSLQPEDVATYYCQKYNSAPLTFGGGTKVEIN

SEQ ID NO: 116 - DNA, 388 bp, Artificial Sequence
  QUEL-0105 VH domain nucleotide
caggtgcaactggtggagtctgggggagacgtggtccagcctgggaggtccctgagactctcctgtgcagcctctggtttcaccttcaaaaactatggcatgcactgggtccgccagggtctaggcaaggggctggagtgggtggctattatatcatatgatggaacaactaaatactctgtagactccgtgaagggccgattcaccatctccagagacaattccaagaacacgttgtatctgcaaatgaacagcctgagaactgaggacacggctctgtattactgtgcgaaagaggcgggggatttctatggttcggggaattatgggtactactactatggtttggacgtctggggccaagggaccacggtcaccgtctcctcag

SEQ ID NO: 117 - protein, 129 aa, Artificial Sequence
  QUEL-0105 VH domain amino acid
QVQLVESGGDVVQPGRSLRLSCAASGFTFKNYGMHWVRQGLGKGLEWVAIISYDGTTKYSVDSVKGRFTISRDNSKNTLYLQMNSLRTEDTALYYCAKEAGDFYGSGNYGYYYYGLDVWGQGTTVTVSS

SEQ ID NO: 118 - protein, 8 aa, Artificial Sequence
  QUEL-0105 HCDR1
GFTFKNYG

SEQ ID NO: 119 - protein, 22 aa, Artificial Sequence
  QUEL-0105 HCDR3
AKEAGDFYGSGNYGYYYYGLDV

SEQ ID NO: 120 - DNA, 322 bp, Artificial Sequence
  QUEL-0105 VL domain nucleotide
gacatccagatgacccagtctccatcctccctgtctgcatctgtaggagacagagtcaccatcacttgccgggcgaatcaggacattagcaattatttagcctggtatcagcagaaaccagggaaatttcctaagctcctgatctatgctgcatccactttgcaatcaggggtcccatctcggttcagtggcagtggatctgggacagatttcactctcaccatcagcagcctgcagcctgaagatgttgcaacttattactgtcaaaagtataacagtgccccgctcactttcggcggagggaccaaggtggagatcaaac

SEQ ID NO: 121 - protein, 107 aa, Artificial Sequence
  QUEL-0105 VL domain amino acid
DIQMTQSPSSLSASVGDRVTITCRANQDISNYLAWYQQKPGKFPKLLIYAASTLQSGVPSRFSGSGSGTDFTLTISSLQPEDVATYYCQKYNSAPLTFGGGTKVEIK

SEQ ID NO: 122 - DNA, 355 bp, Artificial Sequence
  QUEL-0302 VH domain nucleotide
gaggtgcagctggtggagtctgggggaggcttggtccagcctggggggtccctgagactctcctgtgcagcctctggattcacctttagtaactcttggatgaactgggtccgccaagctccagggaaggggctggagtgggtggccaacataaaccaagatggcagtgagagatactatgtggactctgtgaagggccgattcaccatctccagagacaactccaagaactcactgtctctgcaaatgaacagcctgagagccgaggacacggctatctattactgtgcgagagaaataactggaacctactttgactactggggccagggaaccctggtcaccgtctcctcag

SEQ ID NO: 123 - protein, 118 aa, Artificial Sequence
  QUEL-0302 VH domain amino acid
EVQLVESGGGLVQPGGSLRLSCAASGFTFSNSWMNWVRQAPGKGLEWVANINQDGSERYYVDSVKGRFTISRDNSKNSLSLQMNSLRAEDTAIYYCAREITGTYFDYWGQGTLVTVSS

SEQ ID NO: 124 - protein, 8 aa, Artificial Sequence
  QUEL-0302 HCDR2
INQDGSER

SEQ ID NO: 125 - protein, 11 aa, Artificial Sequence
  QUEL-0302 HCDR3
AREITGTYFDY

SEQ ID NO: 126 - DNA, 331 bp, Artificial Sequence
  QUEL-0302 VL domain nucleotide
cagtctgtgctgactcagccaccctcagcgtctgggacccccgggcagagggtcaccatctcttgttctggaagcagctccaacatcggaaataattatgtatactggtaccaacagctcccaggaacggcccccaaactcctcatctataggaataatcagcggccctcaggggtccctgaccgattctctggctccaagtctggcacctcagcctccctggccatcagtgggctccggtccgaggatgaggctgaatattactgtgcagcatgggatgacagcctgagtggtccggtgttcggcggagggaccaagctgaccgtcctag

SEQ ID NO: 127 - protein, 110 aa, Artificial Sequence
  QUEL-0302 VL domain amino acid
QSVLTQPPSASGTPGQRVTISCSGSSSNIGNNYVYWYQQLPGTAPKLLIYRNNQRPSGVPDRFSGSKSGTSASLAISGLRSEDEAEYYCAAWDDSLSGPVFGGGTKLTVL

SEQ ID NO: 128 - protein, 8 aa, Artificial Sequence
  QUEL-0302 LCDR1
SSNIGNNY

SEQ ID NO: 129 - skipped

SEQ ID NO: 130 - protein, 11 aa, Artificial Sequence
  QUEL-0302 LCDR3
AAWDDSLSGPV

SEQ ID NO: 131 - DNA, 355 bp, Artificial Sequence
  QUEL-0303 VH domain nucleotide
gaggtgcagctggtggagtctgggggaggcttggtccagcctggggggtccctgagactctcctgtgcagcctctggattcacctttagtaactattggatgaactgggtccgccaggctccagggaaggggctggagtgggtggccaacatagaccaagatggaagtgagaaatactatgtggactctttgaagggccgattcaccatctccagagacaacgccaagaactcactgtatctgcaaatgaacagcctgagagccgaggactcggctgtgtattactgtgcgagagagctaactggaactacttttgactactggggccagggaaccctggtcaccgtctcctcag

SEQ ID NO: 132 - protein, 118 aa, Artificial Sequence
  QUEL-0303 VH domain amino acid
EVQLVESGGGLVQPGGSLRLSCAASGFTFSNYWMNWVRQAPGKGLEWVANIDQDGSEKYYVDSLKGRFTISRDNAKNSLYLQMNSLRAEDSAVYYCARELTGTTFDYWGQGTLVTVSS

SEQ ID NO: 133 - protein, 8 aa, Artificial Sequence
  QUEL-0303 HCDR1, QUEL-0304 HCDR1
GFTFSNYW

SEQ ID NO: 134 - protein, 8 aa, Artificial Sequence
  QUEL-0303 HCDR2
IDQDGSEK

SEQ ID NO: 135 - protein, 11 aa, Artificial Sequence
  QUEL-0303 HCDR3
ARELTGTTFDY

SEQ ID NO: 136 - DNA, 331 bp, Artificial Sequence
  QUEL-0303 VL domain nucleotide
cagtctgtgctgactcagccaccctcagcgtctgggacccccgggcagagggtcaccatctcttgttctggaagcagctccaacatcggaagtaatactataaactggtaccaactgctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggtccggtgttcggcggagggaccaagctgaccgtcctag

SEQ ID NO: 137 - protein, 110 aa, Artificial Sequence
  QUEL-0303 VL domain amino acid
QSVLTQPPSASGTPGQRVTISCSGSSSNIGSNTINWYQLLPGTAPKLLIYSNNQRPSGVPDRFSGSKSGTSASLAISGLQSEDEADYYCAAWDDSLNGPVFGGGTKLTVL

SEQ ID NO: 138 - protein, 8 aa, Artificial Sequence
  QUEL-0303 LCDR1, QUEL-0304 LCDR1
SSNIGSNT

SEQ ID NO: 139 - protein, 11 aa, Artificial Sequence
  QUEL-0303 LCDR3
AAWDDSLNGPV

SEQ ID NO: 140 - DNA, 355 bp, Artificial Sequence
  QUEL-0304 VH domain nucleotide
gaggtgcagctggtggagtctgggggaggcttggtccagcctggggggtccctgagactctcctgtgcagcctctggattcacatttagtaactattggatgagctgggtccgccaggctccagggaaggggctggagtgggtggccaacatgaaccatgatggaagtgataaatactatgtggactctgtgaagggccgattcaccatctccagagacaacgccaagagtttaatgtatctgcaaatgaacagcctgagagccgaggacacggctgtatattactgtgcgagagatcggactggaactatctttgactactggggccagggaaccttggtcaccgtctcctcag

SEQ ID NO: 141 - protein, 118 aa, Artificial Sequence
  QUEL-0304 VH domain amino acid
EVQLVESGGGLVQPGGSLRLSCAASGFTFSNYWMSWVRQAPGKGLEWVANMNHDGSDKYYVDSVKGRFTISRDNAKSLMYLQMNSLRAEDTAVYYCARDRTGTIFDYWGQGTLVTVSS

SEQ ID NO: 142 - protein, 8 aa, Artificial Sequence
  QUEL-0304 HCDR2
MNHDGSDK

SEQ ID NO: 143 - protein, 11 aa, Artificial Sequence
  QUEL-0304 HCDR3
ARDRTGTIFDY

SEQ ID NO: 144 - DNA, 331 bp, Artificial Sequence
  QUEL-0304 VL domain nucleotide
cagtctgtgctgactcagccaccctcagcgtctgggacccccgggcagagggtcaccatctcttgttctggaagcagctccaacatcggaagtaatactgtaaactggtaccagcaactcccaggaacggcccccaaagtcctcatctatagtaataatcagcggccctcaggggtccctgaccgattctctggctccaagtctggcacctcagcctccctggccatcagtgggctccagtctgaggatgaggctaattattactgtgcaacatgggatgacagcctgaatggtccggtgttcggcggagggaccaagctgaccgtcctag

SEQ ID NO: 145 - protein, 110 aa, Artificial Sequence
  QUEL-0304 VL domain amino acid
QSVLTQPPSASGTPGQRVTISCSGSSSNIGSNTVNWYQQLPGTAPKVLIYSNNQRPSGVPDRFSGSKSGTSASLAISGLQSEDEANYYCATWDDSLNGPVFGGGTKLTVL

SEQ ID NO: 146 - protein, 11 aa, Artificial Sequence
  QUEL-0304 LCDR3
ATWDDSLNGPV

(19) United States
(12) Patent Application Publication
(10) Pub. No.: US 2024/0182585 A1
(43) Pub. Date: Jun. 6, 2024

(54) THERAPEUTIC INHIBITORS OF GDF15 SIGNALLING

(71) Applicants: Cambridge Enterprise Limited, Cambridge, Cambridgeshire (GB); KYMAB LIMITED, Cambridge (GB)

(72) Inventors: Stephen O’RAHILLY, Cambridge, Cambridgeshire (GB); Anthony Patrick COLL, Cambridge, Cambridgeshire (GB); Irene CIMINO, Cambridge, Cambridgeshire (GB); E-Chiang LEE, Cambridge (GB); Hui LIU, Cambridge (GB); Adam CARPENTER, Cambridge (GB)

(21) Appl. No.: 18/284,952

(22) PCT Filed: Mar. 31, 2022
(86) PCT No.: PCT/EP2022/058669

§ 371 Date: Sep. 29, 2023

(30) Foreign Application Priority Data
Mar. 31, 2021 (GB) 2104556.2
May 21, 2021 (GB) 2107331.7
Jun. 8, 2021 (GB) 2108170.8

(51) Int. Cl. C07K 16/28; A61K 39/00; A61P 21/00

(52) U.S. Cl. CPC C07K 16/2863; A61P 21/00; A61K 2039/505; C07K 2317/33; C07K 2317/52; C07K 2317/565; C07K 2317/76; C07K 2317/92

(57) ABSTRACT

This invention relates to antibodies that bind to and inhibit the activity of glial cell-derived neurotrophic factor family receptor alpha like (GFRAL) protein. The invention also relates to the GDF15-GFRAL signalling pathway as a therapeutic target for states of cachexia and conditions involving reduction in food intake and reduction in muscle and fat mass.

### FIELD OF THE INVENTION

This invention relates to antibodies that bind to and inhibit the activity of glial cell-derived neurotrophic factor family receptor alpha like (GFRAL) protein. The invention also relates to the GDF15-GFRAL signalling pathway as a therapeutic target for states of cachexia and conditions involving reduction in food intake and reduction in muscle and fat mass.

### BACKGROUND

The cell surface receptor known as “glial cell-derived neurotrophic factor family receptor alpha like” is encoded by the GFRAL gene, which is located on human chromosome 6p12.1 and has 9 exons encoding a sequence of 394 amino acids.

In the developing mouse brain, the mRNA level of Gfral in the cerebral cortex and hippocampus has been shown to reach a maximum at birth and decline afterwards, indicating that it might take part in neuroprotection and brain development [.1]. In the adult mouse, Gfral transcripts have been found primarily in the central nervous system. Its expression has been found to be restricted to certain neurons of the brainstem, specifically in the area postrema (AP) and the nucleus of the solitary tract (nucleus tractus solitariusu, NTS). Later on, Gfral expression was also detected at low levels in testis and adipose tissue [.2, .3, .4, .5].

The ligand for GFRAL has been identified as the hormone GDF15 (growth and differentiation factor 15) [2, 3, 4, 5, .6]. Upon interaction with GDF15, the GDF15-GFRAL complex interacts with RET, a tyrosine protein kinase receptor, and induces cellular signalling through activation of MAPK, AKT and PLC-γ signalling pathways. This signalling has been shown to have the downstream effect of regulating food intake, energy expenditure and body weight [2, 3, 4, 5]. The active signalling complex is an assembly of six polypeptides, comprising a homodimer of GDF15-GFRAL-RET.

GDF15 is a distant member of the TGF-β family. It is a stress-induced cytokine and can be upregulated by tissue injury [.7], ionising radiation [.8], hypoxia [.9], inflammation [.10], chemical toxins [42, 43] and various disease states, including cancer [.11], rheumatoid arthritis [.12], chronic renal failure [.13], cardiovascular diseases [.14], obesity and diabetes [.15]. It is also induced by tissue-damaging toxins such as chemotherapeutic agents [.16, .17]. It is a stress-responsive cytokine that drives down appetite/food intake, as demonstrated in mouse [.18] and non-human primate models [4]. Circulating levels of GDF15 have been reported to be elevated in a broad spectrum of conditions, including many disease states [42, 44]. GDF15 is believed to represent a “sentinel” hormone response to cellular stress, its protective effects including limiting systemic exposure to recently ingested toxins (e.g., through emesis) and, through its induction of conditioned aversion, promoting the avoidance of future exposures to agents which have previously led to cellular stress [42].

High levels of GDF15 are linked with cachexia in some cancer patients. The presence of cancer in the body may increase the level of GDF15, driving cancer cachexia. Cachexia is a multifactorial disease characterised by weight loss via skeletal muscle and adipose tissue loss, an imbalance in metabolic regulation, and reduced food intake [.19]. Cachexia is estimated to affect up to 74% of patients with many types of cancer globally, with the highest incidence in head and neck, pancreatic, gastric, and hepatic cancer [.20]. Cancer cachexia not only negatively affects the quality of life of patients with cancer [.21, .22], but also reduces the effectiveness of anti-cancer chemotherapy [.23, .24] and increases its toxicity [.25, .26, .27], leading to increased cancer-related mortality [27, .28, .29] and increased expenditure of medical resources. A range of conditions other than cancer are also associated with cachexia, including pulmonary and cardiac conditions (e.g., congestive heart failure, chronic obstructive pulmonary disease), as well as chronic kidney disease, acquired immune deficiency syndrome (AIDS), and the advanced states of cystic fibrosis, multiple sclerosis, motor neuron disease, Parkinson's disease, dementia, tuberculosis, multiple system atrophy, mercury poisoning, Crohn's disease, rheumatoid arthritis and celiac disease.

In cachectic animal models, administration of monoclonal antibodies against GDF15 led to increased food intake, better locomotor function and energy expenditure, and was able to reverse weight loss and restore skeletal muscle [.30, .31]. Blocking the GDF15-GFRAL pathway reversed body weight loss caused by cancer cachexia and extended survival [30]. An anti-GFRAL antagonist antibody that inhibits the RET signalling complex in brainstem neurons was reported to reverse excessive lipid oxidation and prevent cancer cachexia when injected into tumour-bearing mice [.32]. In 2019, a clinical trial was initiated with anti-GFRAL monoclonal antibody NGM120 in the treatment of cancer anorexia/cachexia syndrome (NCT04068896).

Gfral^{-/-} (“knock-out”) mice on a chow diet appear of normal body weight, and they become obese on a high-fat diet just like wild-type mice. According to some reports, GfraIGFRAL knock-out mice become slightly heavier than wild-type mice on a high fat diet. Wild-type mice treated with GDF15 exhibit significantly attenuated food intake and sustained weight loss in comparison to vehicle-treated mice, whereas Gfral^{-/-} mice are refractory to the effects of GDF15 [3, 4]. This illustrates the suppression of body weight gain by GDF15-GFRAL signalling. Further, GDF15 overexpression has been shown to protect mice from the development of obesity and improve their glucose tolerance on a high-fat diet [11, .33]. In mice or rats that are fed chow or high-fat diet, GDF15 administration reduces food intake and body weight. Cynomolgus monkeys with spontaneous obesity show decreased food intake resulting in significant weight loss after 4 weeks of exposure to recombinant HAS-GDF15 [2, 3, 4, 5, 6]. In a rat model, serum GDF15 level was positively correlated with tumour volume and negatively correlated with food intake, and it was shown that GDF15 induces anorexia through nausea and emesis [.34, .35].

There are also reports that the GDF15-GFRAL pathway is highly associated with hyperemesis gravidarum [.36].

Thus, signalling induced by the GDF15-GFRAL-RET complex regulates food intake and body weight, and its activity plays an important role in conditions such as cancer cachexia. By inhibiting the activity of the GDF15-GFRAL-RET complex, for example by inhibiting formation of this complex at the cell surface, antagonists offer a potential route to therapeutic treatment of these and other conditions. Activation of the hypothalamic-pituitary-adrenal (HPA) axis by exogenous and endogenous GDF15 was described by Cimino et al., PNAS 118 (27) 2021, the content of which is incorporated herein by reference.

### SUMMARY OF THE INVENTION

The present invention provides antibodies to human GFRAL. We generated and selected antibodies against the GFRAL protein, which inhibit the activity of GFRAL with high potency, antagonising its downstream signalling. These antibodies exhibit high affinity binding to GFRAL on the surface of GFRAL-expressing cells, and inhibit GDF15-induced intracellular signalling in GFRAL-expressing cells. Antibody binding to the GFRAL extracellular domain may inhibit formation of the GDF15-GFRAL-RET complex, e.g., by inhibiting association of RET with GDF15-GFRAL. We selected antibodies displaying high affinity and high potency in assays with human GFRAL, combined with cross-reactivity for non-human GFRAL, e.g., mouse GFRAL.

Exemplary antibodies of the present invention are named QUEL-0101, QUEL-0201 and QUEL-0301. Further, related antibodies of the present invention are named QUEL-0102, QUEL-0103, QUEL-0104, QUEL-0105, QUEL-0302, QUEL-0303 and QUEL-0304. The present invention extends to anti-GFRAL binding molecules incorporating antigen-binding sequences of QUEL-0101, QUEL-0201 or QUEL-0301, or of any of QUEL-0102, QUEL-0103, QUEL-0104, QUEL-0105, QUEL-0302, QUEL-0303 or QUEL-0304, such as their complementarity determining regions (CDRs), e.g., heavy chain CDRs (HCDRs) HCDR1, HCDR2 and/or HCDR3 and/or light chain CDRs (LCDRs) LCDR1, LCDR2 and/or LCDR3 and optionally the heavy and/or light chain variable (VH and/or VL) domain, and variants thereof. A binding molecule may comprise the HCDR1, HCDR2 and/or HCDR3 of QUEL-0101, QUEL-0102, QUEL-0103, QUEL-0104, QUEL-0105, QUEL-0201, QUEL-0301, QUEL-0302, QUEL-0303 or QUEL-0304 in a polypeptide scaffold and/or it may comprise the LCDR1, LCDR2 and/or LCDR3 of QUEL-0101, QUEL-0102, QUEL-0103, QUEL-0104, QUEL-0105, QUEL-0201, QUEL-0301, QUEL-0302, QUEL-0303 or QUEL-0304 in a polypeptide scaffold. For example, the binding molecule may comprise the HCDR3 of QUEL-0201, optionally the QUEL-0201 set of HCDRs, and optionally it may comprise the HCDRs and LCDRs of QUEL-0201.

Embodiments of the invention include antibodies comprising a VH domain and a VL domain, the VH domain comprising a set of HCDRs HCDR1, HCDR2 and HCDR3 and the VL domain comprising a set of LCDRs LCDR1, LCDR2 and LCDR3, wherein
(i) the set of HCDRs is the QUEL-0101 set of HCDRs defined wherein HCDR1 is SEQ ID NO: 3, HCDR2 is SEQ ID NO: 4 and HCDR3 is SEQ ID NO: 5,
(ii) the set of HCDRs is the QUEL-0201 set of HCDRs defined wherein HCDR1 is SEQ ID NO: 13, HCDR2 is SEQ ID NO: 14 and HCDR3 is SEQ ID NO: 15, or
(iii) the set of HCDRs is the QUEL-0301 set of HCDRs defined wherein HCDR1 is SEQ ID NO: 23, HCDR2 is SEQ ID NO: 24 and HCDR3 is SEQ ID NO: 25.

Further embodiments of the invention include antibodies comprising a VH domain and a VL domain, the VH domain comprising a set of HCDRs HCDR1, HCDR2 and HCDR3 and the VL domain comprising a set of LCDRs LCDR1, LCDR2 and LCDR3, wherein
(iv) the set of HCDRs is the QUEL-0102 set of HCDRs defined wherein HCDR1 is SEQ ID NO: 99, HCDR2 is SEQ ID NO: 100 and HCDR3 is SEQ ID NO: 101,
(v) the set of HCDRs is the QUEL-0103 set of HCDRs defined wherein HCDR1 is SEQ ID NO: 107, HCDR2 is SEQ ID NO: 108 and HCDR3 is SEQ ID NO: 5,
(vi) the set of HCDRs is the QUEL-0104 set of HCDRs defined wherein HCDR1 is SEQ ID NO: 107, HCDR2 is SEQ ID NO: 108 and HCDR3 is SEQ ID NO: 113, or
(vii) the set of HCDRs is the QUEL-0105 set of HCDRs defined wherein HCDR1 is SEQ ID NO: 118, HCDR2 is SEQ ID NO: 108 and HCDR3 is SEQ ID NO: 119.

Further embodiments of the invention include antibodies comprising a VH domain and a VL domain, the VH domain comprising a set of HCDRs HCDR1, HCDR2 and HCDR3 and the VL domain comprising a set of LCDRs LCDR1, LCDR2 and LCDR3, wherein
(viii) the set of HCDRs is the QUEL-0302 set of HCDRs defined wherein HCDR1 is SEQ ID NO: 23, HCDR2 is SEQ ID NO: 124 and HCDR3 is SEQ ID NO: 125,
(ix) the set of HCDRs is the QUEL-0303 set of HCDRs defined wherein HCDR1 is SEQ ID NO: 133, HCDR2 is SEQ ID NO: 134 and HCDR3 is SEQ ID NO: 135, or
(x) the set of HCDRs is the QUEL-0304 set of HCDRs defined wherein HCDR1 is SEQ ID NO: 133, HCDR2 is SEQ ID NO: 142 and HCDR3 is SEQ ID NO: 143.

An antibody of the present invention may comprise a VH domain and a VL domain,
the VH domain comprising a set of HCDRs HCDR1, HCDR2 and HCDR3, and
the VL domain comprising a set of LCDRs LCDR1, LCDR2 and LCDR3, wherein
HCDR1 is SEQ ID NO: 3, SEQ ID NO: 99, SEQ ID NO: 107 or SEQ ID NO: 118,
HCDR2 is SEQ ID NO: 4, SEQ ID NO: 100 or SEQ ID NO: 108 and
HCDR3 is SEQ ID NO: 5, SEQ ID NO: 101, SEQ ID NO: 113 or SEQ ID NO: 119, and optionally wherein
LCDR1 is SEQ ID NO: 8,
LCDR2 is SEQ ID NO: 9 and
LCDR3 is SEQ ID NO: 10 or SEQ ID NO: 104.

An antibody of the present invention may comprise a VH domain and a VL domain,
the VH domain comprising the QUEL-0101 set of HCDRs HCDR1 SEQ ID NO: 3, HCDR2 SEQ ID NO: 4 and HCDR3 SEQ ID NO: 5, and
the VL domain comprising the QUEL-0101 set of LCDRs LCDR1 SEQ ID NO: 8, LCDR2 SEQ ID NO: 9 and LCDR3 SEQ ID NO: 10.

In another embodiment, the antibody comprises a VH domain and a VL domain,
the VH domain comprising the QUEL-0102 set of HCDRs HCDR1 SEQ ID NO: 99, HCDR2 SEQ ID NO: 100 and HCDR3 SEQ ID NO: 101, and
the VL domain comprising the QUEL-0102 set of LCDRs LCDR1 SEQ ID NO: 8, LCDR2 SEQ ID NO: 9 and LCDR3 SEQ ID NO: 104.

In another embodiment, the antibody comprises a VH domain and a VL domain,
the VH domain comprising the QUEL-0103 set of HCDRs HCDR1 SEQ ID NO: 107, HCDR2 SEQ ID NO: 108 and HCDR3 SEQ ID NO: 5, and
the VL domain comprising the QUEL-0103 set of LCDRs LCDR1 SEQ ID NO: 8, LCDR2 SEQ ID NO: 9 and LCDR3 SEQ ID NO: 104.

In another embodiment, the antibody comprises a VH domain and a VL domain,
the VH domain comprising the QUEL-0104 set of HCDRs HCDR1 SEQ ID NO: 107, HCDR2 SEQ ID NO: 108 and HCDR3 SEQ ID NO: 113, and
the VL domain comprising the QUEL-0104 set of LCDRs LCDR1 SEQ ID NO: 8, LCDR2 SEQ ID NO: 9 and LCDR3 SEQ ID NO: 104.

In another embodiment, the antibody comprises a VH domain and a VL domain,
the VH domain comprising the QUEL-0105 set of HCDRs HCDR1 SEQ ID NO: 118, HCDR2 SEQ ID NO: 108 and HCDR3 SEQ ID NO: 119, and
the VL domain comprising the QUEL-0105 set of LCDRs LCDR1 SEQ ID NO: 8, LCDR2 SEQ ID NO: 9 and LCDR3 SEQ ID NO: 104. An antibody of the present invention may comprise the QUEL-0101 VH domain SEQ ID NO: 2 or a variant thereof in which there are one or more amino acid alterations and/or the QUEL-0101 VL domain SEQ ID NO: 7 in which there are one or more amino acid alterations. An amino acid alteration may be a substitution, deletion or insertion of an amino acid. Such alterations may optionally be in the variable domain framework, outside the CDRs. For example, there may be one or two amino acid alterations in the VH and/or VL domain framework. Alterations may be conservative substitutions and/or may represent germlining of framework residues. To germline an amino acid residue means to revert it to the residue that occurs at that position as encoded by the germline gene segment from which the variable domain was produced. Corresponding germline gene segments may be identified by comparing the variable domain sequence against human v, d and j (for VH) or human v and j (for VL) gene segments and identifying the closest match. Variants of the QUEL-0101 VH domain include VH domains that have one or more alterations that are present in the QUEL-0102, QUEL-0103, QUEL-0104 or QUEL-0105 VH domain, e.g., that are present in the framework of one or more of these VH domains. An antibody of the present invention optionally comprises the QUEL-0102 VH domain SEQ ID NO: 98, the QUEL-0103 VH domain SEQ ID NO: 106, the QUEL-0104 VH domain SEQ ID NO: 112 or the QUEL-0105 VH domain SEQ ID NO: 117.

The VH domain may be encoded by a nucleotide sequence produced by recombination of heavy chain v gene segment IGHV3-30 (e.g., IGHV3-30*18) with a d gene segment and a heavy chain j gene segment, e.g., IGHJ6 (e.g., IGHJ6*02). Thus, it may comprise a VH domain framework produced by recombination of IGHV3-30 and IGHJ6 (“an IGHV3-30 IGHJ6 framework”). Optionally, the VH domain is produced by recombination of IGHV3-30, IGHD3-10 and IGHJ6.

The antibody may comprise a VH domain having at least 90% amino acid sequence identity to the QUEL-0101 VH domain SEQ ID NO: 2, optionally at least 95%, at least 98% or at least 99% sequence identity. The VH domain may comprise or consist of SEQ ID NO: 2. The VH domain may comprise or consist of a VH domain encoded by SEQ ID NO: 1 expressed in a mammalian cell, e.g., CHO.

The VL domain may be encoded by a nucleotide sequence produced by recombination of light chain v gene segment IGKV1-27 (e.g., IGKV1-27*01) with a light chain j gene segment, e.g., a kappa j segment such as IGKJ4 (e.g., IGKJ4*01). Thus, it may comprise a VL domain framework produced by recombination of IGKV1-27 and IGKJ4 (“an IGKV1-27 IGKJ4 framework”).

The antibody may comprise a VL domain having at least 90% amino acid sequence identity to the QUEL-0101 VH domain SEQ ID NO: 7, optionally at least 95%, at least 98% or at least 99% sequence identity. The VL domain may comprise or consist of SEQ ID NO: 7. The VL domain may comprise or consist of a VL domain encoded by SEQ ID NO: 6 expressed in a mammalian cell, e.g., CHO.

An anti-GFRAL antibody of the invention may comprise the QUEL-0101 VH domain SEQ ID NO: 2 and the QUEL-0101 VL domain SEQ ID NO: 7.

In another embodiment, it comprises the QUEL-0102 VH domain SEQ ID NO: 98 and the QUEL-0102 VL domain SEQ ID NO: 103.

In another embodiment, it comprises the QUEL-0103 VH domain SEQ ID NO: 106 and the QUEL-0103 VH domain SEQ ID NO: 110.

In another embodiment, it comprises the QUEL-0104 VH domain SEQ ID NO: 112 and the QUEL-0104 VL domain SEQ ID NO: 115.

In another embodiment, it comprises the QUEL-0105 VH domain SEQ ID NO: 117 and the QUEL-0105 VH domain SEQ ID NO: 121.

An antibody of the present invention may comprise a VH domain and a VL domain,
the VH domain comprising the QUEL-0201 set of heavy chain complementarity determining regions (HCDRs) HCDR1 SEQ ID NO: 13, HCDR2 SEQ ID NO: 14 and HCDR3 SEQ ID NO: 15, and
the VL domain comprising the QUEL-0201 set of light chain complementarity determining regions (LCDRs) LCDR1 SEQ ID NO: 18, LCDR2 SEQ ID NO: 19 and LCDR3 SEQ ID NO: 20.

An antibody of the present invention may comprise the QUEL-0201 VH domain SEQ ID NO: 12 or a variant thereof in which there are one or more amino acid alterations and/or the QUEL-0201 VL domain SEQ ID NO: 17 in which there are one or more amino acid alterations. An amino acid alteration may be a substitution, deletion or insertion of an amino acid. Such alterations may optionally be in the variable domain framework, outside the CDRs. For example, there may be one or two amino acid alterations in the VH and/or VL domain framework. Alterations may be conservative substitutions and/or may represent germlining of framework residues.

The VH domain may be encoded by a nucleotide sequence produced by recombination of heavy chain v gene segment IGHV1-3 (e.g., IGHV1-3*01) with a d gene segment and a heavy chain j gene segment, e.g., IGHJ6 (e.g., IGHJ6*02). Thus, it may comprise a VH domain framework produced by recombination of IGHV1-3 and IGHJ6 (“an IGHV1-3 IGHJ6 framework”). Optionally, the VH domain is produced by recombination of IGHV1-3, IGHD5-18 and IGHJ6.

The antibody may comprise a VH domain having at least 90% amino acid sequence identity to the QUEL-0201 VH domain SEQ ID NO: 12, optionally at least 95%, at least 98% or at least 99% sequence identity. The VH domain may comprise or consist of SEQ ID NO: 12. The VH domain may comprise or consist of a VH domain encoded by SEQ ID NO: 11 expressed in a mammalian cell, e.g., CHO.

The VL domain may be encoded by a nucleotide sequence produced by recombination of light chain v gene segment IGLV1-40 (e.g., IGLV1-40*01) with a light chain j gene segment, e.g., a lambda j segment such as IGLJ3 (e.g., IGLJ3*02). Thus, it may comprise a VL domain framework produced by recombination of IGLV1-40 and IGLJ3 (“an IGLV1-40 IGLJ3 framework”).

An antibody of the present invention may comprise the QUEL-0201 VL domain SEQ ID NO: 17 or a variant thereof having one or more amino acid alterations. The antibody may comprise a VL domain having at least 90% amino acid sequence identity to the QUEL-0201 VH domain SEQ ID NO: 17, optionally at least 95%, at least 98% or at least 99% sequence identity. The VL domain may comprise or consist of SEQ ID NO: 17. The VL domain may comprise or consist of a VL domain encoded by SEQ ID NO: 16 expressed in a mammalian cell, e.g., CHO.

An anti-GFRAL antibody of the invention may comprise the QUEL-0201 VH domain SEQ ID NO: 12 and the QUEL-0201 VL domain SEQ ID NO: 17.

An antibody of the present invention may comprise a VH domain and a VL domain,
the VH domain comprising a set of HCDRs HCDR1, HCDR2 and HCDR3, and
the VL domain comprising a set of LCDRs LCDR1, LCDR2 and LCDR3, wherein
HCDR1 is SEQ ID NO: 23 or SEQ ID NO: 133,
HCDR2 is SEQ ID NO: 24, SEQ ID NO: 124, SEQ ID NO 134 or SEQ ID NO: 142,
HCDR3 is SEQ ID NO: 25, SEQ ID NO: 125, SEQ ID NO: 135 or SEQ ID NO: 143, and optionally wherein
LCDR1 is SEQ ID NO: 28, SEQ ID NO: 128 or SEQ ID NO: 138,
LCDR2 is SEQ ID NO: 29 or SEQ ID NO: 129, and
LCDR3 is SEQ ID NO: 30, SEQ ID NO: 130, SEQ ID NO: 139 or SEQ ID NO: 146.

An antibody of the present invention may comprise a VH domain and a VL domain,
the VH domain comprising the QUEL-0301 set of heavy chain complementarity determining regions (HCDRs) HCDR1 SEQ ID NO: 23, HCDR2 SEQ ID NO: 24 and HCDR3 SEQ ID NO: 25, and
the VL domain comprising the QUEL-0301 set of light chain complementarity determining regions (LCDRs) LCDR1 SEQ ID NO: 28, LCDR2 SEQ ID NO: 29 and LCDR3 SEQ ID NO: 30.

In another embodiment, the antibody comprises a VH domain and a VL domain, the VH domain comprising the QUEL-0302 set of HCDRs HCDR1 SEQ ID NO: 23, HCDR2 SEQ ID NO: 124 and HCDR3 SEQ ID NO: 125, and
the VL domain comprising the QUEL-0302 set of LCDRs LCDR1 SEQ ID NO: 128, LCDR2 SEQ ID NO: 129 and LCDR3 SEQ ID NO: 130.

In another embodiment, the antibody comprises a VH domain and a VL domain, the VH domain comprising the QUEL-0303 set of HCDRs HCDR1 SEQ ID NO: 133, HCDR2 SEQ ID NO: 134 and HCDR3 SEQ ID NO: 135, and
the VL domain comprising the QUEL-0303 set of LCDRs LCDR1 SEQ ID NO: 138, LCDR2 SEQ ID NO: 29 and LCDR3 SEQ ID NO: 139.

In another embodiment, the antibody comprises a VH domain and a VL domain, the VH domain comprising the QUEL-0304 set of HCDRs HCDR1 SEQ ID NO: 133, HCDR2 SEQ ID NO: 142 and HCDR3 SEQ ID NO: 143, and
the VL domain comprising the QUEL-0304 set of LCDRs LCDR1 SEQ ID NO: 138, LCDR2 SEQ ID NO: 29 and LCDR3 SEQ ID NO: 146.

An antibody of the present invention may comprise the QUEL-0301 VH domain SEQ ID NO: 22 or a variant thereof in which there are one or more amino acid alterations and/or the QUEL-0301 VL domain SEQ ID NO: 27 in which there are one or more amino acid alterations. An amino acid alteration may be a substitution, deletion or insertion of an amino acid. Such alterations may optionally be in the variable domain framework, outside the CDRs. For example, there may be one or two amino acid alterations in the VH and/or VL domain framework. Alterations may be conservative substitutions and/or may represent germlining of framework residues. Variants of the QUEL-0301 VH domain include VH domains that have one or more alterations that are present in the QUEL-0302, QUEL-0303 or QUEL-0304 VH domain, e.g., that are present in the framework of one or more of these VH domains. An antibody of the present invention optionally comprises the QUEL-0302 VH domain SEQ ID NO: 123, the QUEL-0303 VH domain SEQ ID NO: 132 or the QUEL-0304 VH domain SEQ ID NO: 141.

The VH domain may be encoded by a nucleotide sequence produced by recombination of heavy chain v gene segment IGHV3-7 (e.g., IGHV3-7*01) with a d gene segment and a heavy chain j gene segment, e.g., IGHJ4 (e.g., IGHJ4*02). Thus, it may comprise a VH domain framework produced by recombination of IGHV3-7 and IGHJ4 (“an IGHV3-7 IGHJ4 framework”). Optionally, the VH domain is produced by recombination of IGHV3-7, IGHD1-7 and IGHJ4. Alternatively, the VH domain is produced by recombination of IGHV3-7, IGHD1-20 and IGHJ4.

The antibody may comprise a VH domain having at least 90% amino acid sequence identity to the QUEL-0301 VH domain SEQ ID NO: 22, optionally at least 95%, at least 98% or at least 99% sequence identity. The VH domain may comprise or consist of SEQ ID NO: 22. The VH domain may comprise or consist of a VH domain encoded by SEQ ID NO: 21 expressed in a mammalian cell, e.g., CHO.

The VL domain may be encoded by a nucleotide sequence produced by recombination of light chain v gene segment IGLV1-44 (e.g., IGLV1-44*01) or IGLV1-47 (e.g., IGLV1-47*01) with a light chain j gene segment, e.g., a lambda j segment such as IGLJ3 (e.g., IGLJ3*02). In one embodiment, the light chain v gene segment is IGLV1-44. In another embodiment, the light chain v gene segment is IGLV1-47. Thus, it may comprise a VL domain framework produced by recombination of IGLV1-44 and IGLJ3 (“an IGLV1-44 IGLJ3 framework”). Alternatively, it may comprise a VL domain framework produced by recombination of IGLV1-47 and IGLJ3 (“an IGLV1-47 IGLJ3 framework”).

The antibody may comprise a VL domain having at least 90% amino acid sequence identity to the QUEL-0301 VL domain SEQ ID NO: 27, optionally at least 95%, at least 98% or at least 99% sequence identity. The VL domain may comprise or consist of SEQ ID NO: 27. The VL domain may comprise or consist of a VL domain encoded by SEQ ID NO: 26 expressed in a mammalian cell, e.g., CHO.

An anti-GFRAL antibody of the invention may comprise the QUEL-0301 VH domain SEQ ID NO: 22 and the QUEL-0301 VL domain SEQ ID NO: 27.

In another embodiment, it comprises the QUEL-0302 VH domain SEQ ID NO: 123 and the QUEL-0302 VL domain SEQ ID NO: 127.

In another embodiment, it comprises the QUEL-0303 VH domain SEQ ID NO: 132 and the QUEL-0303 VL domain SEQ ID NO: 137.

In another embodiment, it comprises the QUEL-0304 VH domain SEQ ID NO: 141 and the QUEL-0304 VL domain SEQ ID NO: 145.

An anti-GFRAL antibody according to the present invention may be one that competes for binding to human GFRAL with QUEL-0101, QUEL-0201 or QUEL-0301. For example, it may compete with QUEL-0201 IgG comprising QUEL-0201 VH domain SEQ ID NO: 12 and QUEL-0201 VL domain comprising SEQ ID NO: 17. Alternatively, it may compete with QUEL-0101 IgG comprising QUEL-0101 VH domain SEQ ID NO: 2 and QUEL-0101 VL domain SEQ ID NO: 7 and/or it may compete with QUEL-0301 IgG comprising QUEL-0301 VH domain SEQ ID NO: 22 and QUEL-0301 VL domain SEQ ID NO: 27. The ability of a binding molecule to compete with a reference molecule for binding to GFRAL may be determined in vitro, e.g., by surface plasmon resonance (SPR) in a sandwich assay.

An anti-GFRAL antibody according to the present invention may be one which does not compete with GDF15 for binding to GFRAL. Ability of a binding molecule to compete with GDF15 for binding to GFRAL may be also determined by SPR sandwich assay.

High affinity binding to GFRAL is advantageous. An anti-GFRAL antibody preferably binds human GFRAL with an affinity (K_{D}) of 1 nanomolar (nM) or stronger (i.e., 1 nM or less than 1 nM) considering the limited expression of GRFAL protein. Affinity may be determined by SPR, e.g., as described in Example 2. The K_{D} may be 0.5 nM (500 picomolar, pM) or less, 400 pM or less, 300 pM or less, 200 pM or less, or 100 pM or less. The K_{D} may be 50 pM or less, e.g., 10 pM or less. The K_{D} may be approximately 100 pM, e.g., in the range 50 pM to 200 pM. The K_{D} may be 5 pM or less, 4 pM or less, 3 pM or less, 2 pM or less or 1 pM or less. The K_{D} may be approximately 1 pM (e.g., 0.5 pM-2 pM). In some embodiments, K_{D} may be at least 0.1 pM. The K_{D} may be at least 0.5 pM. The K_{D} may be at least 1 pM, e.g., in the range 1 pM to 1 nM.

An anti-GFRAL antibody preferably also binds non-human GFRAL (e.g., mouse GFRAL, rat GFRAL and/or cynomolgus GFRAL) in addition to human GFRAL. Optimally, an antibody will be cross-reactive for mouse and cynomolgus GFRAL (within 10-fold affinity/potency). The amino acid sequence identity between human GFRAL (FIG. 1) and mouse GFRAL (FIG. 2) is 70%, and between human and cynomolgus monkey is 94%. The degree of cross-reactivity of an antibody may be quantified as a fold difference in K_{D} for binding human and non-human GFRAL. Preferably, an antibody will have an affinity for mouse GFRAL within 100-fold, within 10-fold, within 5-fold or more preferably within 2-fold of its affinity for human GFRAL.

An anti-GFRAL antibody may bind mouse GFRAL with an affinity (K_{D}) of 10 nM or stronger, preferably 5 nM or stronger. Affinity for mouse GFRAL is optionally 100 pM or less.

Anti-GFRAL antibodies described herein are inhibitors of GDF15 signalling. Specifically, they inhibit GDF15 signalling through GFRAL, by binding to GFRAL extracellular domain and inhibiting formation of the cell surface GDF15-GFRAL-RET signalling complex. Potency of inhibition by anti-GFRAL antagonist antibodies may be determined in an in vitro assay, such as an ERK phosphorylation assay. This is a cell-based assay which determines the ability of a candidate antagonist molecule to inhibit the RET signalling that is triggered by addition of GDF15. Potency can be quantified as IC50 in the assay. Preferably, an antibody inhibits GFRAL activation with a potency (IC50) of 15 nM or stronger (i.e., 15 nM or less than 15 nM) in the ERK phosphorylation assay. Preferably, IC50 is 10 nM or less, e.g., 5 nM or less.

Nucleic acid encoding antibodies as described herein is also provided, as are cells comprising said nucleic acid. A host cell in vitro may comprise the nucleic acid, optionally integrated into its cellular (e.g., genomic) DNA, or transiently transfected (e.g., plasmid DNA).

Anti-GFRAL antagonist antibodies are suitable for medical use. Antagonistic antibodies that target GFRAL, inhibiting signalling of the hormone GDF15 via the GDF15-GFRAL-RET pathway, represent potential therapeutic agents for conditions such as cachexia (e.g., cancer cachexia), and hyperemesis gravidarum. Inhibition of the GDF15:GFRAL interaction, exemplified herein with antagonistic anti-GFRAL antibody, inhibits the action of GDF15 on food intake and body weight in vivo. Anti-GFRAL antibodies, or their encoding nucleic acid, may be administered to patients to increase food intake (e.g., for patients with cachexia or anorexia relating to cancer). Anti-GFRAL antibodies or their encoding nucleic acids may be used as an adjuvant therapeutic drug, in combination with other anti-cancer interventions such as surgery, radiotherapy and/or administration of anti-neoplastic drugs. We demonstrate herein that antagonistic anti-GFRAL antibodies can inhibit production of corticosterone induced by GDF15 in mice. Corticosterone is a steroid hormone in the glucocorticoid class in mice. The human equivalent of corticosterone is cortisol. Cortisol levels in plasma are increased in response to a wide range of stressors. Cortsiol suppresses the immune system, promotes catabolism of protein, alters lipolysis differentially in different adipose tissue depots and promotes gluconeogenesis. Glucocorticoids are essential for life. However if their circulating levels are excessive for extended periods they can have detrimental effects including promoting muscle wasting. Cachexia is a condition characterised by a reduction in lean mass as well as fat mass. In several conditions associated with cachexia there is evidence of chronic activation of the hypothalamic pituitary adrenal (HPA) axis [37, 38, 39]. Given the known effects of glucocorticoids it is likely that the excess activation of the HPA axis is playing a contributory role in the loss of lean mass in conditions characterised by cachexia. The work we present herein suggests that the blockade of GDF15 signalling at its receptor GFRAL will inhibit the pathological activation of the HPA axis by elevated levels of GDF15 and thus reduce any adverse effects of the chronic elevation of glucocorticoids on lean mass, including skeletal muscle.

Further aspects of the invention therefore relate to treatment of conditions where elevated levels of GDF15 promote excessive activation of the hypothalamic pituitary adrenal axis (HPA) leading to adverse effects of pathologically elevated levels of circulating cortisol in a patient, wherein the treatment comprises administering an inhibitor of GDF15 signalling to the patient. An inhibitor of GDF15 signalling may reduce cortisol the circulating levels of cortisol (e.g., as measured in blood plasma or by assessment of the excretion of free cortisol in urine).

These and other aspects and embodiments of the invention, including methods of producing antibodies, pharmaceutical compositions, and methods of treating patients, are described in more detail below.

### BRIEF DESCRIPTION OF THE DRAWINGS

FIG. 1 shows the amino acid sequence of human GFRAL SEQ ID NO: 31, NCBI NP_997293.2, comprising 394 amino acids of which residues 1-18 represent a signal peptide. Boxed asparagine residues are sites of N-linked glycosylation (GlcNAc) at N23, N50, N62, N67, N103 and N116. GFRAL contains two GDNF/GAS1 domains, formed by residues 131-210 and 220-316 respectively, boxed. A region believed to be required for interaction with GDF15 is residues 149-228, italicised. A transmembrane domain spans residues 352-371, emboldened. Polymorphic variants of residue 33, underlined, exist in the human population and according to the 1000 genomes project residue R is present at ˜62% and C is present at ˜38%. Residue P386 in the cytoplasmic tail is also variable and may be S.

FIG. 2 shows the amino acid sequence of mouse GFRAL SEQ ID NO: 32, NCBI NP_995316.2. Sequence feature annotations use the same convention as for FIG. 1.

FIG. 3 shows ΔF % plotted against log human GDF15 molar concentration in an ERK phosphorylation assay.

FIG. 4 shows change in fluorescence for antagonist titrations in the ERK phosphorylation assay with 4 nM (0.05 μg/ml) GDF15.

FIG. 5 shows sensorgrams from SPR analysis of antibodies binding to human GFRAL. (A) QUEL-0101. (B) QUEL-0201. (C) QUEL-0301.

FIG. 6 shows sensorgrams from SPR analysis of antibodies binding to mouse GFRAL. (A) QUEL-0101. (B) QUEL-0201. (C) QUEL-0301.

FIG. 7 shows the ability of anti-GFRAL antibodies to inhibit GDF15-induced reduction in (A) food intake and (B) body weight. Data are for treatment groups from left to right: isotype control; low dose QUEL-0301; low dose QUEL-0201; high dose QUEL-0301; high dose QUEL-0201.

FIG. 8 presents data from Mouse Study 15 (MS15) relating to validation of GFRAL blocking antibody QUEL-0201 in mice. Percentage change in (AA) food intake and (BB) body weight in the 24 h following control vehicle (left bar) or human recombinant GDF15 (right bar) administration. Data are expressed as mean±SEM, n=6-8 per group. **p<0.01, ***p<0.001, ****p<0.0001 by ANOVA.

FIG. 9 shows data from Mouse Study 3 (MS3): corticosterone serum level in QUEL-0201- and IgG control-treated mice with (right bar) or without (left bar) 0.1 mg/kg human recombinant GDF15 administration. Data are expressed as mean±SEM, n=6-8 per group. **p<0.01, ***p<0.001, ****p<0.0001 by ANOVA.

FIG. 10 shows data from MS3:: human GDF15 serum concentration in QUEL-0201 and IgG groups treated with human recombinant GDF15. Data are expressed as mean±SEM, n=6-8 per group. **p<0.01, ***p<0.001, ****p<0.0001 by unpaired Student's t-test.

FIG. 11A-D shows data from Example 8 in muscles (A) tibialis anterior; (B) gastrocnemius; (C) extensor digitorum longus; (D) soleus. Relative fold change in mRNA expression level of Mafbx, Murf1 and Foxo1 (normalised to beta-2 microglobulin) in QUEL-0201- or IgG control-treated mice given either 0.1 mg/kg human recombinant GDF15 administration (right bar, black square) or sham injection (left bar, white circle). Data are expressed as mean±SEM, n=4-5 per group. *p<0.05, **p<0.01, ***p<0.001, ****p<0.0001 by two-way ANOVA with multiple comparison.

### DETAILED DESCRIPTION

### Role of GDF15-GFRAL-RET Signalling in Activation of the HPA Axis

The activation of the hypothalamic-pituitary-adrenal (HPA) axis, which results in an increase in circulating levels of glucocorticoids occurs in response to a wide range of stressful stimuli. Glucocorticoid hormones (in humans, predominantly cortisol) have actions on inflammation, metabolism and blood vessels that help the organism to withstand life-threatening challenges [.40]. In response to infections, pro-inflammatory cytokines such as TNFα/β, IL-1 and IL-6 activate the axis [.41].

Studies of cellular responses to chemical toxins have frequently identified GDF15 as one of the most highly upregulated genes [42, .43]. GDF15 is ubiquitously produced in the body, with circulating concentrations rising rapidly upon exposure to a wide variety of stressors [42, 44]. Cisplatin is known to elevate circulating levels of both corticosterone (the rodent equivalent of cortisol) [.45, .46] and GDF15 [35, 35, .47]. Endoplasmic reticulum (ER) stress is mechanistically distinct from genotoxicity and its effects to increase GDF15 expression and secretion are well established [48, .49]. Actions resulting from GDF15-GFRAL-RET signalling have largely focused on regulation of food intake, anorexia, cachexia, emesis and conditioned aversion, as discussed in the Background section herein. This range of actions would be consistent with GDF15 playing a role in signalling the presence of chemical threats to the organism which might be mitigated by reduced rate of exposure to, or expulsion of, ingested toxins and the promotion of their avoidance in future.

As reported herein, we discovered that antagonism of GFRAL, using anti-GFRAL antibody, both (i) inhibits the action of GDF15 on food intake and body weight in vivo, and also, at the same dose, (ii) inhibits an increase in circulating glucocorticoid levels in response to GDF15. Thus, we show that inhibiting GFRAL inhibits a neuroendocrine response comprising an effect of GDF15 on raising glucocorticoid levels. Anti-GFRAL counters the GDF15-induced increase in circulating glucocorticoid.

### Inhibitors of GDF15 Signalling

Inhibitors of GDF15 signalling include molecules that inhibit activation of the GDF15-GFRAL-RET signalling complex. An inhibitor may inhibit formation of this complex (e.g., by inhibiting GDF15 binding to GFRAL, or by inhibiting GDF15-GFRAL binding to RET) and/or its functional activity (e.g., by biasing the receptor to a less active conformation). Inhibition of GDF15 signalling is optionally measured in an in vitro assay such as the ERK phosphorylation assay described herein. Other inhibitors of GDF15 signalling may reduce levels of GDF15, GFRAL or RET, e.g., by downregulating their expression or increasing their degradation. An inhibitor may downregulate GFRAL or RET by reducing its presence on the cell surface.

An inhibitor optionally binds to GDF15, GFRAL or RET, or to its encoding nucleic acid. The inhibitor may optionally be a small molecule, a nucleic acid (e.g., an inhibitory RNA), or a binder polypeptide. A binder polypeptide is a polypeptide molecule with an ability to specifically bind and inhibit its target antigen (e.g., GDF15, GFRAL or RET).

Many classes of binder polypeptides are known in the art, including classical IgG antibodies and other binding proteins based on immunoglobulin domains. Non-immunoglobulin binding molecules are also known, and binding loops may be engineered into other polypeptide scaffolds such as fibronectin.

Preferably, a binder polypeptide of the present invention comprises an immunoglobulin domain in which a binding site for the antigen is formed by loop regions of the immunoglobulin domain. Preferred embodiments of binder polypeptides are antibodies. Examples of anti-GFRAL antibodies are described in detail herein.

Other GDF15 signalling inhibitors, including e.g., anti-GFRAL and anti-GDF15 antibodies, are described in WO2017/189724, WO2017/172260, WO2017/152105, WO2017/147742, WO2017/121865, WO2017/055613, WO2016/049470, WO2015/144855, WO2014/100689, WO2014/049087, WO2013/023557, WO2013/012648, WO2011/070177, WO2005/099746 and WO2005/072112.

### Therapeutic Use

Aspects of the present invention include:
methods of treating patients with an inhibitor of GDF15 signalling,
an inhibitor of GDF15 signalling for use in treating patients, and
use of an inhibitor of GDF15 signalling for the manufacture of a medicament for treating patients.

An inhibitor of GDF15 signalling as described herein, e.g., an anti-GFRAL antibody, may be used to treat any medical condition associated with excessive activation of the GDF15-GFRAL pathway. Examples of such conditions are described herein, and include
(i) cachexia (loss of appetite and body weight including fat and lean mass, including skeletal muscle) due to a wide range of conditions including cancer, heart failure, renal failure, chronic, chronic lung disease and rare genetic diseases such as mitochondrial myopathies and the thalassemias
(ii) Neuroendocrine dysfunction resulting from high GDF15 levels in all of the conditions listed in paragraph (i) above
(iii) Nausea and vomiting as a result of agents that rapidly increase GDF15, such as cancer chemotherapy
(iv) Hyperemesis gravidarum

An inhibitor of GDF15 signalling at GFRAL may be used to treat any condition where the levels of GDF15 are acutely or chronically elevated.

Acute elevation of GDF15, as occurs in response to stimuli such as cytotoxic chemotherapy, acute exposure to ionizing radiation or in the first trimester of pregnancy, is known to be associated with nausea, vomiting and reduced physical activity and activation of the hypothalamic pituitary adrenal axis. Blockade of GDF15 signalling at GFRAL is predicted to reduce all of these phenomena.

Chronic elevations of GDF15 occurs in a wide variety of conditions including cancer, chronic heart failure, chronic respiratory illness, chronic renal failure and a range of rare diseases such as the thalassemias and thalassemia mitochondrial myopathy. In all of these conditions, elevated GDF15 is associated with chronically reduced appetite and food intake, loss of fat mass and loss of muscle mass all of which together is referred to as cachexia. In at least some forms of cachexia there is evidence for a chronic excess activation of the HPA axis. Given the known effects of cortisol on muscle protein breakdown, it is at least plausible that preventing the excessive activation of the HPA axis will have beneficial effects on the loss of muscle mass.

Treatment may comprise preventative treatment, wherein the therapeutic composition is administered in advance of emergence of the condition to be treated, in order to prevent or at least ameliorate the effects of the condition. Treatment may also be given after emergence of the condition, e.g., the treatment may be prescribed following diagnosis of the condition. The conditions listed above may be side effects of other medical interventions such as treatment with cytotoxic agents (e.g., anti-neoplastic agents used in treating cancer) or radiotherapy.

Treatment may be directed towards reducing elevation of cortisol in a patient. The patient may have elevated circulating levels of cortisol, detectable by sampling at different times of the day in the blood or urine (e.g., by measuring 24 hour urine free cortisol [50]).

The patient may have an elevated physiological level of GDF15, detectable in the blood. Human serum levels of GDF15 are reported to be between 150 to 1150 pg/mL in one study [.51]. Methods of measuring GDF15 from blood samples have been described, e.g., an ELISA or sandwich immunoassay such as the Elecsys® GDF-15 immunoassay for the in vitro quantitative determination of GDF-15 in human serum and plasma.

The patient to be treated may be a cancer patient. As is evident from the published literature in the field, many types of cancer are associated with an increase in GDF15, which will drive cancer cachexia. The cancer may be a solid tumour (optionally metastatic) or a blood cancer. The cancer may be bladder cancer, brain cancer, breast cancer, colorectal cancer, head and neck cancer, kidney cancer, lung cancer (e.g., non-small cell lung cancer), lymphoma (e.g., non-Hodgkin lymphoma), melanoma, mesothelioma, neuroblastoma, oesophageal cancer, oral or oropharyngeal cancer, gastrointestinal cancer (e.g., gastric cancer), pancreatic cancer, prostate cancer, testicular cancer, thyroid cancer or uterine cancer.

Alternatively, treatment may comprise treating cachexia in a patient with a pulmonary and/or cardiac condition (e.g., congestive heart failure, chronic obstructive pulmonary disease), chronic kidney disease, acquired immune deficiency syndrome (AIDS), cystic fibrosis, multiple sclerosis, motor neuron disease, Parkinson's disease, dementia, tuberculosis, multiple system atrophy, mercury poisoning, Crohn's disease, rheumatoid arthritis or celiac disease.

Treatment may result in reduction of the condition, and increased food intake and/or stabilisation or gain of weight by the patient may be observed. For example, a patient may be treated for cancer-associated cachexia, wherein treatment increases body weight or reduces loss of body weight.

A patient treated in accordance with the present invention may be one who has been exposed or will be exposed to a cytotoxic chemical agent. A patient treated in accordance with the present invention may be one who has been exposed or will be exposed to ionising radiation.

The cytotoxic agent may be an anti-neoplastic agent, examples of which are presented below and elsewhere herein. Treatment may comprise combination therapy, in which a composition of the present invention is administered to a patient who is also receiving treatment with an anti-neoplastic agent, such as cancer chemotherapy. The composition of the present invention may be administered before or after the anti-neoplastic agent, or simultaneously. It will generally be administered in a separate formulation, although a single medicament comprising both agents is a possibility where they can be formulated together. Treatment according to the present invention may enhance the effect of the chemotherapy. The treatment may extend survival of the patient. It is also expected to significantly improve patients' quality of life.

The patient to be treated may be one who has received, or who will receive, treatment with one or more of the following cancer chemotherapeutic agents:
Alkylating agents (e.g., bifunctional alkylators such as cyclophosphamide, mechlorethamine, chlorambucil, melphalan, or monofunctional alkylators such as dacarbazine, nitrosoureas, temozolomide);
Anthracyclines (e.g., daunorubicin, doxorubicin, epirubicin, idarubicin, mitoxantrone, valrubicin;
Cytoskeletal disruptors (e.g., taxanes such as paclitaxel, docetaxel, abraxane, taxotere)
Epothilones (e.g., epothilone A, B, C, D, E or F)
Histone deacetylase inhibitors (e.g., vorinostat, romidepsin)
Inhibitors of topoisomerase I (e.g., irinotecan, topotecan, tafluposide)
Inhibitors of topoisomerase II (e.g., etoposide, teniposide, tafluposide)
Kinase inhibitors (e.g., bortezomib, erlotinib, gefitinib, imatinib, vemurafenib, vismodegib) Nucleotide analogs and precursor analogs (e.g., azacitidine, azathioprine, capecitabine, cytarabine, doxifluridine, fluorouracil, gemcitabine, hydroxyurea, mercaptopurine, methotrexate, tioguanine)
Peptide antibiotics (e.g., bleomycin, actinomycin)
Platinum-based agents (e.g., carboplatin, cisplatin, oxaliplatin)
Retinoids (e.g., alitretinoin, bexarotene, tretinoin)
Vinca alkaloids and derivatives (e.g., vinblastine, vincristine, vindesine, vinorelbine).

### Antibodies

Antibodies according to the present invention are immunoglobulins or molecules comprising immunoglobulin domains, whether natural or partly or wholly synthetically produced. Antibodies may be IgG, IgM, IgA, IgD or IgE molecules or antigen-specific antibody fragments thereof (including, but not limited to, a Fab, F(ab′)2, Fv, disulphide linked Fv, scFv, single domain antibody, closed conformation multispecific antibody, disulphide-linked scfv, diabody), whether derived from any species that naturally produces an antibody, or created by recombinant DNA technology; whether isolated from serum, B-cells, hybridomas, transfectomas, yeast or bacteria. Antibodies can be humanised using routine technology. The term antibody covers any polypeptide or protein comprising an antibody antigen-binding site. An antigen-binding site (paratope) is the part of an antibody that binds to and is complementary to the epitope of its target antigen, e.g., GFRAL.

The term “epitope” refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulphonyl groups, and, in certain embodiments, may have specific three-dimensional structural characteristics, and/or specific charge characteristics.

The antigen binding site is a polypeptide or domain that comprises one or more CDRs of an antibody and is capable of binding the antigen. For example, the polypeptide comprises a CDR3 (e.g., HCDR3). For example the polypeptide comprises CDRs 1 and 2 (e.g., HCDR1 and 2) or CDRs 1-3 of a variable domain of an antibody (e.g., HCDRs1-3).

An antibody antigen-binding site may be provided by one or more antibody variable domains. In an example, the antibody binding site is provided by a single variable domain, e.g., a heavy chain variable domain (VH domain) or a light chain variable domain (VL domain). In another example, the binding site comprises a VH/VL pair or two or more of such pairs. Thus, an antibody antigen-binding site may comprise a VH and a VL.

The antibody may be a whole immunoglobulin, including constant regions, or may be an antibody fragment. An antibody fragment is a portion of an intact antibody, for example comprising the antigen binding and/or variable region of the intact antibody. Examples of antibody fragments include:
(i) a Fab fragment, a monovalent fragment consisting of the VL, VH, CL and CH1 domains; (ii) a F(ab′)2 fragment, a bivalent fragment including two Fab fragments linked by a disulphide bridge at the hinge region;
(iii) an Fd fragment consisting of the VH and CH1 domains;
(iv) an Fv fragment consisting of the VL and VH domains of a single arm of an antibody,
(v) a dAb fragment, which consists of a VH or VL domain; and
(vi) an isolated complementarity determining region (CDR) that retains specific antigen-binding functionality.

Further examples of antibodies are H2 antibodies that comprise a dimer of a heavy chain (5′-VH-(optional hinge)-CH2-CH3-3′) and are devoid of a light chain.

Single-chain antibodies (e.g., scFv) are a commonly used fragment. Multispecific antibodies may be formed from antibody fragments. An antibody of the invention may employ any such format, as appropriate.

Optionally, binder polypeptides, or antibody immunoglobulin domains thereof, may be fused or conjugated to additional polypeptide sequences and/or to labels, tags, toxins or other molecules. Binder polypeptides may be fused or conjugated to one or more different antigen binding regions, providing a molecule that is able to bind a second antigen in addition to GFRAL. For example, an antibody of the present invention may be a multispecific antibody, e.g., a bispecific antibody, comprising (i) an antibody antigen binding site for GFRAL and (ii) a further antigen binding site (optionally an antibody antigen binding site, as described herein) which recognises another antigen.

An antibody normally comprises an antibody VH and/or VL domain. Isolated VH and VL domains of antibodies are also part of the invention. The antibody variable domains are the portions of the light and heavy chains of antibodies that include amino acid sequences of complementarity determining regions (CDRs; ie., CDR1, CDR2, and CDR3), and framework regions (FRs). Thus, within each of the VH and VL domains are CDRs and FRs. A VH domain comprises a set of HCDRs, and a VL domain comprises a set of LCDRs. VH refers to the variable domain of the heavy chain. VL refers to the variable domain of the light chain. Each VH and VL is typically composed of three CDRs and four FRs, arranged from amino-terminus to carboxy-terminus in the following order: FR1, CDR1, FR2, CDR2, FR3, CDR3, FR4. According to the methods used in this invention, the amino acid positions assigned to CDRs and FRs are defined according to IMGT nomenclature.

An antibody may comprise an antibody VH domain comprising a VH CDR1, CDR2 and CDR3 and a framework. It may alternatively or also comprise an antibody VL domain comprising a VL CDR1, CDR2 and CDR3 and a framework. Examples of antibody VH and VL domains and CDRs according to the present invention are as listed in Table A. All VH and VL sequences, CDR sequences, sets of CDRs and sets of HCDRs and sets of LCDRs disclosed herein represent aspects and embodiments of the invention. As described herein, a “set of CDRs” comprises CDR1, CDR2 and CDR3. Thus, a set of HCDRs refers to HCDR1, HCDR2 and HCDR3, and a set of LCDRs refers to LCDR1, LCDR2 and LCDR3. Unless otherwise stated, a “set of CDRs” includes HCDRs and LCDRs.

As described in more detail in the Examples, we isolated and characterised antibodies of particular interest, designated QUEL-0101, QUEL-0201 and QUEL-0301. Subsequently, we identified structural variants of QUEL-0101 and QUEL-0301 respectively, namely QUEL-0102, QUEL-0103, QUEL-0104, QUEL-0105, QUEL-0302, QUEL-0303 and QUEL-0304. In various aspects of the invention, unless context dictates otherwise, an anti-GFRAL antibody may optionally be selected from QUEL-0101, QUEL-0102, QUEL-0103, QUEL-0104, QUEL-0105, QUEL-0201, QUEL-0301, QUEL-0302, QUEL-0303 and QUEL-0304. Optionally, it is selected from QUEL-0101, QUEL-0201 and QUEL-0301.

The present invention encompasses anti-GFRAL antibodies having the VH and/or VL domain sequences of these antibodies, as shown in the appended sequence listing, as well as antibodies comprising the HCDRs and/or LCDRs of those antibodies, and optionally having the full heavy chain and/or full light chain amino acid sequence.

Where an antibody VH domain or VL domain comprises one or more residues in a framework region which differ from the germline gene segment from which it was obtained by recombination, the non-germline residue may be retained or may be mutated to a different residue, e.g., it may be reverted to the germline residue. Corresponding germline gene segments may be identified as the gene segment to which the sequence of the variable domain is most closely aligned, and the germline gene segments corresponding to each of QUEL-0101, QUEL-0201 and QUEL-0301 VH and VL domains, and to their corresponding related antibodies, are shown in Table G herein.

An antibody according to the present invention may comprise one or more CDRs as described herein, e.g. a CDR3, and optionally also a CDR1 and CDR2 to form a set of CDRs. The CDR or set of CDRs may be a CDR or set of CDRs of any of QUEL-0101, QUEL-0102, QUEL-0103, QUEL-0104, QUEL-0105, QUEL-0201, QUEL-0301, QUEL-0302, QUEL-0303 and QUEL-0304.

The invention provides antibodies comprising an HCDR1, HCDR2 and/or HCDR3 of any of antibodies QUEL-0101, QUEL-0102, QUEL-0103, QUEL-0104, QUEL-0105, QUEL-0201, QUEL-0301, QUEL-0302, QUEL-0303 and QUEL-0304 and/or an LCDR1, LCDR2 and/or LCDR3 of any of these antibodies, e.g. a set of CDRs. The antibody may comprise a set of VH CDRs of one of these antibodies. Optionally it may also comprise a set of VL CDRs of one of these antibodies, and the VL CDRs may be from the same or a different antibody as the VH CDRs.

A VH domain comprising a disclosed set of HCDRs, and/or a VL domain comprising a disclosed set of LCDRs, are also provided by the invention.

Typically, a VH domain is paired with a VL domain to provide an antibody antigen-binding site, although as discussed further below a VH or VL domain alone may be used to bind antigen. The QUEL-0201 VH domain may be paired with the QUEL-0201 VL domain, so that an antibody antigen-binding site is formed comprising both the QUEL-0201 VH and VL domains. Analogous embodiments are provided for the other VH and VL domains disclosed herein. In other embodiments, the QUEL-0201 VH is paired with a different VL domain, e.g., a λ VL domain, e.g., the QUEL-0301 VL domain. Conversely, the QUEL-0301 VH may be paired with a different VL domain, e.g., a λ VL domain, e.g., the QUEL-0201 VL domain. Light-chain promiscuity is well established in the art. For example:

The QUEL-0101 VH domain can be paired with the VL domain of any of QUEL-0101, QUEL-0102, QUEL-0103, QUEL-0104 and QUEL-0105.

The QUEL-0102 VH domain can be paired with the VL domain of any of QUEL-0101, QUEL-0102, QUEL-0103, QUEL-0104 and QUEL-0105.

The QUEL-0103 VH domain can be paired with the VL domain of any of QUEL-0101, QUEL-0102, QUEL-0103, QUEL-0104 and QUEL-0105.

The QUEL-0104 VH domain can be paired with the VL domain of any of QUEL-0101, QUEL-0102, QUEL-0103, QUEL-0104 and QUEL-0105.

The QUEL-0105 VH domain can be paired with the VL domain of any of QUEL-0101, QUEL-0102, QUEL-0103, QUEL-0104 and QUEL-0105.

The QUEL-0301 VH domain can be paired with the VL domain of any of QUEL-0301, QUEL-0302, QUEL-0303 and QUEL-0304.

The QUEL-0302 VH domain can be paired with the VL domain of any of QUEL-0301, QUEL-0302, QUEL-0303 and QUEL-0304.

The QUEL-0303 VH domain can be paired with the VL domain of any of QUEL-0301, QUEL-0302, QUEL-0303 and QUEL-0304.

The QUEL-0304 VH domain can be paired with the VL domain of any of QUEL-0301, QUEL-0302, QUEL-0303 and QUEL-0304.

An antibody may comprise one or more CDRs, e.g. a set of CDRs, within an antibody framework. The framework regions may be of human germline gene segment sequences. Thus, the antibody may be a human antibody having a VH domain comprising a set of HCDRs in a human germline framework. Normally the antibody also has a VL domain comprising a set of LCDRs, e.g. in a human germline framework. An antibody “gene segment”, e.g., a VH gene segment, D gene segment, or JH gene segment refers to oligonucleotide having a nucleic acid sequence from which that portion of an antibody is derived, e.g., a VH gene segment is an oligonucleotide comprising a nucleic acid sequence that corresponds to a polypeptide VH domain from FR1 to part of CDR3. Human V, D and J gene segments recombine to generate the VH domain, and human V and J segments recombine to generate the VL domain. The D domain or region refers to the diversity domain or region of an antibody chain. J domain or region refers to the joining domain or region of an antibody chain. Somatic hypermutation may result in an antibody VH or VL domain having framework regions that do not exactly match or align with the corresponding gene segments, but sequence alignment can be used to identify the closest gene segments and thus identify from which particular combination of gene segments a particular VH or VL domain is derived. When aligning antibody sequences with gene segments, the antibody amino acid sequence may be aligned with the amino acid sequence encoded by the gene segment, or the antibody nucleotide sequence may be aligned directly with the nucleotide sequence of the gene segment.

An antibody of the invention may be a human antibody or a chimaeric antibody comprising human variable regions and non-human (e.g., mouse) constant regions. The antibody of the invention for example has human variable regions, and optionally also has human constant regions.

Thus, antibodies optionally include constant regions or parts thereof, e.g., human antibody constant regions or parts thereof. For example, a VL domain may be attached at its C-terminal end to antibody light chain kappa or lambda constant domains. Similarly, an antibody VH domain may be attached at its C-terminal end to all or part (e.g. a CH1 domain or Fc region) of an immunoglobulin heavy chain constant region derived from any antibody isotype, e.g. IgG, IgA, IgE and IgM and any of the isotype sub-classes, such as IgG1 or IgG4. A preferred example is IgG4PE, e.g., SEQ ID NO: 60. Further examples of human heavy chain constant region sequences are shown in Table C.

In a preferred embodiment, the anti-GFRAL antibody is QUEL-0201 IgG comprising
(i) a heavy chain comprising the QUEL-0201 VH domain SEQ ID NO: 12 and human IgG4 heavy chain constant region (e.g., IgG4PE SEQ ID NO: 60), and
(ii) a light chain comprising QUEL-0201 VL domain SEQ ID NO: 17 and a human A constant region (e.g., having an sequence shown in Table C).

In another preferred embodiment, the anti-GFRAL antibody is QUEL-0301 IgG comprising
(i) a heavy chain comprising the QUEL-0301 VH domain SEQ ID NO: 22 and human IgG4 heavy chain constant region (e.g., IgG4PE SEQ ID NO: 60), and
(ii) a light chain comprising QUEL-0301 VL domain SEQ ID NO: 17 and a human A constant region (e.g., having an sequence shown in Table C).

In another preferred embodiment, the anti-GFRAL antibody is QUEL-0101 IgG comprising
(i) a heavy chain comprising the QUEL-0101 VH domain SEQ ID NO: 2 and human IgG4 heavy chain constant region (e.g., IgG4PE SEQ ID NO: 60), and
(ii) a light chain comprising QUEL-0101 VL domain SEQ ID NO: 7 and a human K constant region (e.g., having an sequence shown in Table C).

Constant regions of antibodies of the invention may alternatively be non-human constant regions. For example, when antibodies are generated in transgenic animals (examples of which are described elsewhere herein), chimaeric antibodies may be produced comprising human variable regions and non-human (host animal) constant regions. Some transgenic animals generate fully human antibodies. Others have been engineered to generate antibodies comprising chimaeric heavy chains and fully human light chains. Where antibodies comprise one or more non-human constant regions, these may be replaced with human constant regions to provide antibodies more suitable for administration to humans as therapeutic compositions, as their immunogenicity is thereby reduced.

Digestion of antibodies with the enzyme papain, results in two identical antigen-binding fragments, known also as “Fab” fragments, and a “Fc” fragment, having no antigen-binding activity but having the ability to crystallize. “Fab” when used herein refers to a fragment of an antibody that includes one constant and one variable domain of each of the heavy and light chains. The term “Fc region” herein is used to define a C-terminal region of an immunoglobulin heavy chain, including native-sequence Fc regions and variant Fc regions. The “Fc fragment” refers to the carboxy-terminal portions of both H chains held together by disulphides. The effector functions of antibodies are determined by sequences in the Fc region, the region which is also recognised by Fc receptors (FcR) found on certain types of cells. Digestion of antibodies with the enzyme pepsin, results in a F(ab′)2 fragment in which the two arms of the antibody molecule remain linked and comprise two-antigen binding sites. The F(ab′)2 fragment has the ability to crosslink antigen.

“Fv” when used herein refers to the minimum fragment of an antibody that retains both antigen-recognition and antigen-binding sites. This region consists of a dimer of one heavy and one light chain variable domain in tight, non-covalent or covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognise and bind antigen, although at a lower affinity than the entire binding site.

Antibodies disclosed herein may be modified to increase or decrease serum half-life. In one embodiment, one or more of the following mutations: T252L, T254S or T256F are introduced to increase biological half-life of the antibody. Biological half-life can also be increased by altering the heavy chain constant region CH₁ domain or CL region to contain a salvage receptor binding epitope taken from two loops of a CH₂ domain of an Fc region of an IgG, as described in U.S. Pat. Nos. 5,869,046 and 6,121,022, the modifications described therein are incorporated herein by reference. In another embodiment, the Fc hinge region of an antibody or antigen-binding fragment of the invention is mutated to decrease the biological half-life of the antibody or fragment. One or more amino acid mutations are introduced into the CH₂—CH₃ domain interface region of the Fc-hinge fragment such that the antibody or fragment has impaired Staphylococcyl protein A (SpA) binding relative to native Fc-hinge domain SpA binding. Other methods of increasing serum half-life are known to those skilled in the art. Thus, in one embodiment, the antibody or fragment is PEGylated. In another embodiment, the antibody or fragment is fused to an albumin-biding domain, e.g. an albumin binding single domain antibody (dAb). In another embodiment, the antibody or fragment is PASylated (i.e. genetic fusion of polypeptide sequences composed of PAS (XL-Protein GmbH) which forms uncharged random coil structures with large hydrodynamic volume). In another embodiment, the antibody or fragment is XTENylated®/rPEGylated (i.e. genetic fusion of non-exact repeat peptide sequence (Amunix, Versartis) to the therapeutic peptide). In another embodiment, the antibody or fragment is ELPylated (i.e. genetic fusion to ELP repeat sequence (PhaseBio)). These various half-life extending fusions are described in more detail in Strohl, BioDrugs (2015) 29:215-239, which fusions, e.g. in Tables 2 and 6, are incorporated herein by reference.

### Antibody Constant Regions

As discussed above, antibodies can be provided in various isotypes and with different constant regions. The Fc region of antibodies is recognised by Fc receptors and determines the ability of the antibody to mediate cellular effector functions, including antibody-dependent cell-mediated cytotoxicity (ADCC) activity, complement dependent cytotoxicity (CDC) activity and antibody-dependent cell phagocytosis (ADCP) activity. These cellular effector functions involve recruitment of cells bearing Fc receptors to the site of the target cells, resulting in killing of the antibody-bound cell.

In the context of the present invention it is desirable to avoid cellular effector functions such as ADCC, ADCP and/or CDC. Therefore, antibodies according to the present invention may lack Fc effector function, for example they may contain Fc regions that do not mediate ADCC, ADCP and/or CDC, or they may lack Fc regions or lack antibody constant regions entirely. An antibody may have a constant region which is effector null.

An antibody may have a heavy chain constant region that binds one or more types of Fc receptor but does not induce cellular effector functions, i.e., does not mediate ADCC, CDC or ADCP activity. Such a constant region may be unable to bind the particular Fc receptor(s) responsible for triggering ADCC, CDC or ADCP activity.

An antibody may have a heavy chain constant region that does not bind Fcγ receptors, for example the constant region may comprise a Leu235Glu mutation (i.e., where the wild type leucine residue is mutated to a glutamic acid residue), which may be referred to as an “E” mutation, e.g., IgG4-E. Another optional mutation for a heavy chain constant region is Ser228Pro (“P” mutation), which increases stability by reducing Fab arm exchange. A heavy chain constant region may be an IgG4 comprising both the Leu235Glu mutation and the Ser228Pro mutation. This “IgG4-PE” heavy chain constant region is effector null. An alternative effector null human constant region is a disabled IgG1.

IgG4PE is a preferred antibody isotype for the present invention. A binder polypeptide may be an IgG4PE antibody comprising the sequence of an IgG4PE constant region shown in Table C.

Antibody constant regions may be engineered to have an extended half life in vivo. Examples include “YTE” mutations and other half-life extending mutations (Dall'Acqua, Kiener & Wu, JBC 281(33):23514-23524 2006 and WO02/060919, incorporated by reference herein). The triple mutation YTE is a substitution of 3 amino acids in the IgG CH2 domain, these mutations providing tyrosine at residue 252, threonine at residue 254 and glutamic acid at residue 256, numbered according to the EU index of Kabat. As described in the referenced publications, the YTE modification increases the half-life of the antibody compared with the half-life of a corresponding antibody having a human CH2 wild type domain. To provide an increased duration of efficacy in vivo, antibodies of the present invention may include antibody constant regions (e.g., IgG constant regions, e.g., IgG CH2 domains) that have one or more mutations that increase the half life of the antibody compared with the corresponding wild type human constant region (e.g., IgG, e.g., IgG CH2 domain). Half-life may be determined by standard methods, such as are described in WO02/060919.

Further example constant regions are shown in Table C.

### Binding to GFRAL

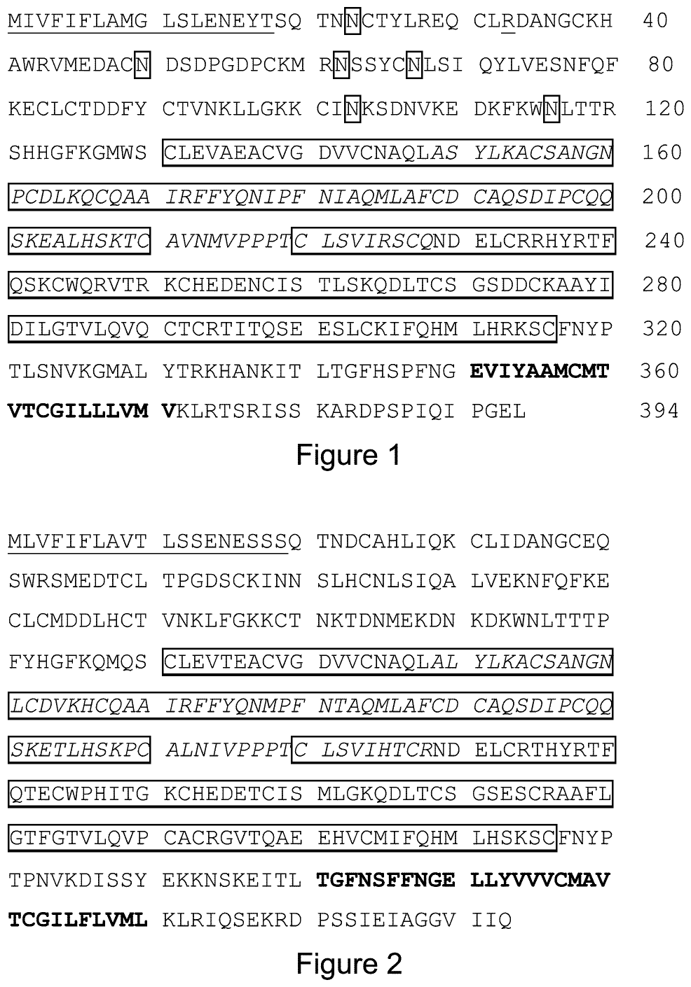

The primary structures of human GFRAL and mouse GFRAL are shown in FIGS. 1 and 2 respectively.

An inhibitor of GDF15 signalling, e.g., an anti-GFRAL antibody, may recognise an epitope within the extracellular domain of GFRAL. It may bind within the sequence of residues 19-351 as shown in FIG. 1. For example, the epitope may comprise an N terminal region of GFRAL comprising residues upstream of the first GDNF/GAS1 domain.

The residues of GFRAL that bind to the antibody (i.e., the precise structural epitope) may be determined by structural resolution of the antibody:antigen complex, e.g., by cryo electron microscopy or by x-ray crystallography. Binding residues may include those that form salt bridges, hydrophobic interactions or hydrogen bonds with the antibody, via their side chain or the polypeptide backbone. The epitope may further include a carbohydrate moiety on the glycosylated antigen.

An anti-GFRAL antibody may recognise an epitope of GFRAL which is the same as or overlaps with the epitope recognised by QUEL-0101, QUEL-0201 or QUEL-0301. An anti-GFRAL antibody may for example bind an epitope comprising one or more, optionally all, of the residues bound by QUEL-0101, QUEL-0201 or QUEL-0301. Recognition of these epitopes is associated with antagonist activity, i.e., inhibition of GDF15-GFRAL-RET signalling, and are thus valuable epitopes to target.

Competition between antibodies or other inhibitors may also be determined. For example, an anti-GFRAL binding agent may compete with an antibody (e.g., IgG or scFv) comprising the VH and VL domains, or an IgG comprising the full heavy and light chains, of QUEL-0101, QUEL-0201 or QUEL-0301. It may for example compete with QUEL-0201 IgG.

Competition between binder polypeptides or other agents indicates that they have epitopes in the same region of GFRAL, e.g., both may bind the same domain with an overlapping binding footprint. This may be confirmed by other techniques, e.g., by structural resolution of the anti-GFRAL molecule bound to GFRAL, which may be achieved for example using cryo electron microscopy or x-ray crystallography as mentioned.

Competition is optionally determined by a sandwich assay to assess the ability of the two binders to simultaneously bind GFRAL extracellular domain in solution. In this assay a first binder (e.g., anti-GFRAL antibody QUEL-0101, QUEL-0201 or QUEL-0301 IgG) is coupled to a solid support and the GFRAL antigen is added in solution, allowing formation of an antibody-antigen (or other binder-antigen) complex. The test antibody or other anti-GFRAL binding molecule is then added in solution. If binding of the test molecule is detected, this indicates that it does not compete with the first binder (e.g., reference (QUEL) antibody). If binding of the test molecule is not detected, this indicates that it does not compete for binding GFRAL with the first binder (e.g., reference antibody). The assay may be performed using SPR, wherein the first binder bound to the surface of a biosensor chip. For an IgG, coupling is commonly via the Fc region, e.g., using a chip coated with anti-Fc antibody. See Example 4 for further details of the SPR sandwich assay.

Optionally, an anti-GFRAL binding agent does not inhibit binding of GDF15 to GFRAL. This may be determined in a sandwich assay using the principles described above, coupling the binder to a solid support and adding GFRAL antigen in solution, then determining binding or absence of binding of GDF15.

### Affinity for Binding

The affinity of a binder (e.g., anti-GFRAL antibody) for GFRAL may be quantified in terms of the equilibrium dissociation constant K_{D}, which is the ratio Ka/Kd of the association or on-rate (Ka) and the dissociation or off-rate (kd) of the binding interaction. K_{D}, Ka and Kd for antigen binding can be measured using surface plasmon resonance SPR. Example SPR procedure and conditions are set out in Example 2 and Example 3. Affinity (K_{D}) is a measure of how strong the interaction of the antibody with its antigen is. Association rate (ka) shows how fast antigen is recognised. Dissociation rate (ka) is a measure of stability of binding. Taken together, kinetic data provide valuable information with implications for biological activity, pharmacokinetics and dosing regimen.

SPR may comprise coating or immobilising the anti-GFRAL binder on to a biosensor chip (directly or indirectly), exposing the binder to the antigen in buffered solution at a range of concentrations, detecting binding, and calculating the equilibrium dissociation constant K_{D} for the binding interaction. For IgG antibodies, coupling to the chip can conveniently be done via Fc capture on an anti-Fc-coated chip (a chip with anti-human Fc antibody on its surface, e.g., chemically immobilised at the chip surface). The binding data can be fitted to a 1:1 model using standard algorithms, which may be inherent to the instrument used. A variety of SPR instruments are known, such as Biacore™, ProteOn XPR36™ (Bio-Rad®), and KinExA® (Sapidyne Instruments, Inc). In brief, SPR may be performed at 25° C. by capturing the binder on a chip for 60 seconds at 1 μg/ml concentration (e.g., approximately between 80 and 140 RU may be captured), and soluble GFRAL (analyte) injected for 120 sec (association time) at 30 μLL/min and dissociation monitored for 1200 seconds. Analyte may be injected at a dilution series, (e.g., 100, 25, 6.25, 1.56, 0.39, 0.098, 0.024 and 0 nM concentration). A suitable running buffer is HBS-P+ buffer pH 7.4 with 1 mM CaCl₂. Sensorgrams for the binder polypeptide are generated, and data may be fitted to a 1:1 interaction model. K_{D} and optionally other kinetic data are calculated. Isolated purified GFRAL extracellular domain may conveniently be used in assays and is a suitable analyte for SPR (see Example 2).

### Species Cross-Reactivity for Binding GFRAL

To enable therapeutic use or testing of anti-GFRAL antibodies in non-human animals, the antibody must be cross-reactive with the corresponding antigen in the species of interest.

Antibodies of the present invention preferably bind mouse GFRAL, rat GFRAL and/or cynomolgus GFRAL in addition to human GFRAL.

The extent of species cross-reactivity of an anti-GFRAL antibody or other anti-GFRAL binding molecule is as the fold-difference in its affinity for antigen or one species compared with antigen of another species, e.g., fold difference in affinity for human antigen vs mouse antigen. Affinity may be quantified as K_{D}, referring to the equilibrium dissociation constant of the binding of the antigen to the antigen-binding molecule. K_{D} may be determined by SPR as described elsewhere herein.

A species cross-reactive binding molecule may have a fold-difference in affinity for binding human and non-human antigen that is 100-fold or less, 50-fold or less, 30-fold or less, 25-fold or less, 20-fold or less, 15-fold or less, 10-fold or less, 5-fold or less, or 2-fold or less. To put it another way, the K_{D} of binding the extracellular domain of the human antigen may be within 100-fold, 50-fold, 30-fold, 25-fold, 20-fold, 15-fold, 10-fold, 5-fold or 2-fold of the K_{D} of binding the extracellular domain of the non-human antigen.

Preferably, the binding affinities of human and non-human antigen are within a range of 10-fold or less, more preferably within 5-fold or within 2-fold. K_{D} for binding mouse GFRAL, e.g., as determined by SPR, may be up to 10-fold (preferably up to 5-fold or up to 2-fold) greater or up to 10-fold lower (preferably up to 5-fold or up to 2-fold lower) than the K_{D} for binding human GFRAL.

Binding molecules can also be considered species cross-reactive if the K_{D} for binding antigen of both species meets a threshold value, e.g., if the K_{D} of binding human antigen and the K_{D} of binding non-human (e.g., mouse) antigen are both 10 mM or less, preferably 5 mM or less, more preferably 1 mM or less. The K_{D} may be 100 nM or less, 50 nM or less, 25 nM or less, 10 nM or less, 5 nM or less, 2 nM or less, or 1 nM or less.

A binding molecule may have a measurable capacity to inhibit GDF15 signalling in a cell based assay with GFRAL from multiple species, (e.g., one or more, or all, of human and mouse, rat and cynomolgus GFRAL). It may exhibit dose-dependent inhibition of GDF15-induced GFRAL signalling activity in an assay described herein with human and non-human (e.g., mouse, rat or cynomolgus) GFRAL, e.g., in the ERK phosphorylation assay.

While species cross-reactivity for binding antigen of different species may be advantageous, selectivity of the binder for GFRAL is nevertheless desirable to avoid unwanted side effects. Thus, within the body, GFRAL is preferably the only antigen bound by the antigen-binding site of the binder polypeptide. Notwithstanding this, a binder polypeptide may optionally be engineered to comprise further binding sites, and an antibody comprising an antibody constant region may for example optionally bind one or more Fc receptors.

### Inhibition of GDF15-GFRAL-RET Signalling Complex

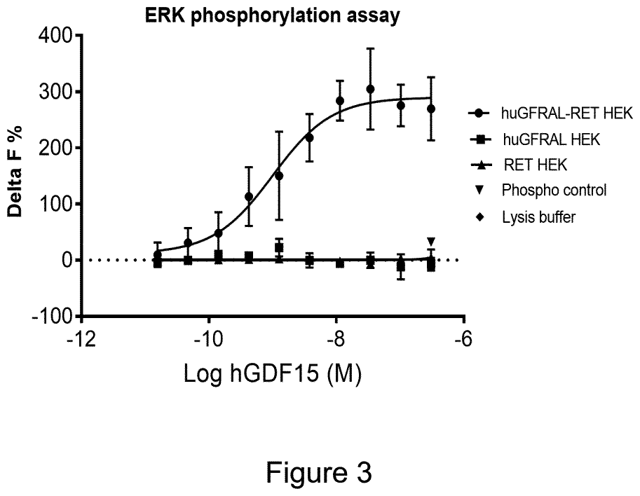

Functional activity of an inhibitor of GDF15 signalling, e.g., an anti-GFRAL antibody, may be tested in vitro. A suitable assay is the ERK phosphorylation assay. In this assay, cells co-expressing GFRAL and RET at their cell surface are incubated with GDF15, resulting in formation of the GDF15-GFRAL-RET signalling complex and consequent downstream signalling including phosphorylation of ERK. ERK phosphorylation may be quantified in lysed cells, e.g., by using HTRF and detecting change in fluorescence. In the absence of inhibitor, addition of GDF15 in a dilution series generates a sigmoid curve (FIG. 3). The effect of adding inhibitor in this assay may be quantified by its effect on the change in fluorescence detected by HTRF. The skilled person will include appropriate controls and will calibrate the assay to establish suitable concentrations of reagents. For example, to determine a concentration of GDF15 sufficient to give maximal response in the assay in the absence of GDF15 inhibitor, the GFRAL/RET expressing cells may be exposed to a range of GDF15 concentrations and inhibition at each concentration determined, before selecting a suitable concentration. For example, we found a concentration of 4 nM produced maximal response in our experiments and thus selected 4 nM as the GDF15 concentration to use for this assay. More details are provided in Example 1.

An inhibitor of GDF15 signalling may exhibit dose-dependent inhibition of GDF15-induced GFRAL signalling activity in the ERK phosphorylation assay, e.g., with human and/or non-human (e.g., mouse, rat or cynomolgus) GFRAL. The ERK phosphorylation assay may be performed with the inhibitor at a range of concentrations, to produce a dose-response curve from which an IC50 value may be calculated. Thus an inhibitor according to the present invention may be identified through its dose-dependent inhibition in such an enzymatic assay.

Potency of the inhibitor may be quantified as IC50.

For example, anti-GFRAL antibody may have an IC50 of 15 nM or less in such an assay. Potency of binder polypeptides such as anti-GFRAL antibodies may be compared for reference against one or more anti-GFRAL antibodies described herein. For example, an antibody comprising the VH and VL domains of QUEL-0101, QUEL-0201 or QUEL-0301 may be used as a reference antibody. The reference antibody may be provided as an IgG. An inhibitor of GDF15 signalling, e.g., an anti-GFRAL antibody according to the present invention, may be one which has an IC50 within 50% or within 10% of the IC50 of QUEL-0101, QUEL-0201 or QUEL-0301 IgG, or it may have an IC50 which is lower than the IC50 of said reference antibody. By “within x % of” it is meant that the IC50 of the test binder polypeptide is no more than x % greater than and no more than x % less than the IC50 of the reference antibody.

An anti-GFRAL antibody may have an IC50 of 10 nM or less in an ERK phosphorylation assay, optionally 5 nM or less.

### Generating and Modifying Binder Polypeptides

Methods for identifying and preparing binder polypeptides, including antibodies, are well known in the art.

For example, antibodies may be generated using laboratory animals such as mice, including transgenic mice (eg, the Kymouse®, Velocimouse®, Omnimouse®, Xenomouse®, HuMab Mouse® or MeMo Mouse®), rats (e.g., the Omnirat®), camelids, sharks, rabbits, chickens or other non-human animals immunised with GFRAL or its encoding nucleic acid, followed optionally by humanisation of the constant regions and/or variable regions to produce human or humanised antibodies. In an example, display technologies can be used, such as yeast, phage or ribosome display, as will be apparent to the skilled person. Standard affinity maturation, e.g., using a display technology, can be performed in a further step after isolation of an antibody lead from a transgenic animal, phage display library or other library. Representative examples of suitable technologies are described in US20120093818 (Amgen, Inc), which is incorporated by reference herein in its entirety, eg, the methods set out in paragraphs [0309] to [0346].

There are many reasons why it may be desirable to create variants of a binder, which include optimising a polypeptide sequence for large-scale manufacturing, facilitating purification, enhancing stability or improving suitability for inclusion in a desired pharmaceutical formulation. Protein engineering work can be performed at one or more target residues in the antibody sequence, e.g., to substituting one amino acid with an alternative amino acid (optionally, generating variants containing all naturally occurring amino acids at this position, with the possible exception of Cys and Met), and monitoring the impact on function and expression to determine the best substitution. It is in some instances undesirable to substitute a residue with Cys or Met, or to introduce these residues into a sequence, as to do so may generate difficulties in manufacturing—for instance through the formation of new intramolecular or intermolecular cysteine-cysteine bonds. Where a lead candidate has been selected and is being optimised for manufacturing and clinical development, it will generally be desirable to change its antigen-binding properties as little as possible, or at least to retain the affinity and potency of the parent molecule. However, variants may also be generated in order to modulate key antibody characteristics such as affinity, cross-reactivity or neutralising potency.

An antibody may comprise a set of H and/or L CDRs of any of the disclosed antibodies with one or more amino acid mutations within the disclosed set of H and/or L CDRs. The mutation may be an amino acid substitution, deletion or insertion. Thus for example there may be one or more amino acid substitutions within the disclosed set of H and/or L CDRs. For example, there may be up to 12, 11, 10, 9, 8, 7, 6, 5, 4, 3 or 2 mutations e.g. substitutions, within the set of H and/or L CDRs. For example, there may be up to 6, 5, 4, 3 or 2 mutations, e.g. substitutions, in HCDR3 and/or there may be up to 6, 5, 4, 3, or 2 mutations, e.g. substitutions, in LCDR3. An antibody may comprise the set of HCDRs, LCDRs or a set of 6 (H and L) CDRs shown for any QUEL antibody herein or may comprise that set of CDRs with one or two conservative substitutions.

One or more amino acid mutations may optionally be made in framework regions of an antibody VH or VL domain disclosed herein. For example, one or more residues that differ from the corresponding human germline segment sequence may be reverted to germline. Human germline gene segment sequences corresponding to VH and VL domains of example anti-GFRAL antibodies are indicated in Table G.

An antibody may comprise a VH domain that has at least 60, 70, 80, 85, 90, 95, 98 or 99% amino acid sequence identity with a VH domain of any of the antibodies shown in the appended sequence listing, and/or comprising a VL domain that has at least 60, 70, 80, 85, 90, 95, 98 or 99% amino acid sequence identity with a VL domain of any of those antibodies. Algorithms that can be used to calculate % identity of two amino acid sequences include e.g. BLAST, FASTA, or the Smith-Waterman algorithm, e.g. employing default parameters. Particular variants may include one or more amino acid sequence alterations (addition, deletion, substitution and/or insertion of an amino acid residue).

Alterations may be made in one or more framework regions and/or one or more CDRs. Variants are optionally provided by CDR mutagenesis. The alterations normally do not result in loss of function, so an antibody comprising a thus-altered amino acid sequence may retain an ability to bind human GFRAL and/or mouse GFRAL. It may retain the same quantitative binding ability as an antibody in which the alteration is not made, e.g. as measured in an assay described herein. The antibody comprising a thus-altered amino acid sequence may have an improved ability to bind and/or inhibit human and/or mouse GFRAL.

Alteration may comprise replacing one or more amino acid residue with a non-naturally occurring or non-standard amino acid, modifying one or more amino acid residue into a non-naturally occurring or non-standard form, or inserting one or more non-naturally occurring or non-standard amino acid into the sequence. Examples of numbers and locations of alterations in sequences of the invention are described elsewhere herein. Naturally occurring amino acids include the 20 “standard” L-amino acids identified as G, A, V, L, I, M, P, F, W, S, T, N, Q, Y, C, K, R, H, D, E by their standard single-letter codes. Non-standard amino acids include any other residue that may be incorporated into a polypeptide backbone or result from modification of an existing amino acid residue. Non-standard amino acids may be naturally occurring or non-naturally occurring.

The term “variant” as used herein refers to a peptide or nucleic acid that differs from a parent polypeptide or nucleic acid by one or more amino acid or nucleic acid deletions, substitutions or additions, yet retains one or more specific functions or biological activities of the parent molecule. Amino acid substitutions include alterations in which an amino acid is replaced with a different naturally-occurring amino acid residue. Such substitutions may be classified as “conservative”, in which case an amino acid residue contained in a polypeptide is replaced with another naturally occurring amino acid of similar character either in relation to polarity, side chain functionality or size. Such conservative substitutions are well known in the art. Substitutions encompassed by the present invention may also be “non-conservative”, in which an amino acid residue which is present in a peptide is substituted with an amino acid having different properties, such as naturally-occurring amino acid from a different group (e.g., substituting a charged or hydrophobic amino; acid with alanine), or alternatively, in which a naturally-occurring amino acid is substituted with a non-conventional amino acid. In some embodiments amino acid substitutions are conservative. Also encompassed within the term variant when used with reference to a polynucleotide or polypeptide, refers to a polynucleotide or polypeptide that can vary in primary, secondary, or tertiary structure, as compared to a reference polynucleotide or polypeptide, respectively (e.g., as compared to a wild-type polynucleotide or polypeptide).

In some aspects, one can use “synthetic variants”, “recombinant variants”, or “chemically modified” polynucleotide variants or polypeptide variants isolated or generated using methods well known in the art. “Modified variants” can include conservative or non-conservative amino acid changes, as described below. Polynucleotide changes can result in amino acid substitutions, additions, deletions, fusions and truncations in the polypeptide encoded by the reference sequence. Some aspects use include insertion variants, deletion variants or substituted variants with substitutions of amino acids, including insertions and substitutions of amino acids and other molecules) that do not normally occur in the peptide sequence that is the basis of the variant, for example but not limited to insertion of ornithine which do not normally occur in human proteins. The term “conservative substitution,” when describing a polypeptide, refers to a change in the amino acid composition of the polypeptide that does not substantially alter the polypeptide's activity. For example, a conservative substitution refers to substituting an amino acid residue for a different amino acid residue that has similar chemical properties (e.g., acidic, basic, positively or negatively charged, polar or nonpolar, etc.). Conservative amino acid substitutions include replacement of a leucine with an isoleucine or valine, an aspartate with a glutamate, or a threonine with a serine. Conservative substitution tables providing functionally similar amino acids are well known in the art. For example, the following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (1), Leucine (L), Methionine (M), Valine (V); and 6) Phenylalanine (F), Tyrosine (Y), Tryptophan (W). (See also Creighton, Proteins, W. H. Freeman and Company (1984), incorporated by reference in its entirety.) In some embodiments, individual substitutions, deletions or additions that alter, add or delete a single amino acid or a small percentage of amino acids can also be considered “conservative substitutions” if the change does not reduce the activity of the peptide. Insertions or deletions are typically in the range of about 1 to 5 amino acids. The choice of conservative amino acids may be selected based on the location of the amino acid to be substituted in the peptide, for example if the amino acid is on the exterior of the peptide and expose to solvents, or on the interior and not exposed to solvents.

One can select the amino acid that will substitute an existing amino acid based on the location of the existing amino acid, including its exposure to solvents (i.e., if the amino acid is exposed to solvents or is present on the outer surface of the peptide or polypeptide as compared to internally localized amino acids not exposed to solvents). Selection of such conservative amino acid substitutions are well known in the art, for example as disclosed in Dordo et al, J. Mol Biol, 1999, 217, 721-739 and Taylor et al, J. Theor. Biol. 119(1986); 205-218 and S. French and B. Robson, J. Mol. Evol. 19(1983)171. Accordingly, one can select conservative amino acid substitutions suitable for amino acids on the exterior of a protein or peptide (i.e. amino acids exposed to a solvent), for example, but not limited to, the following substitutions can be used: substitution of Y with F, T with S or K, P with A, E with D or Q, N with D or G, R with K, G with N or A, T with S or K, D with N or E, I with L or V, F with Y, S with T or A, R with K, G with N or A, K with R, A with S, K or P.

In alternative embodiments, one can also select conservative amino acid substitutions encompassed suitable for amino acids on the interior of a protein or peptide, for example one can use suitable conservative substitutions for amino acids is on the interior of a protein or peptide (i.e. the amino acids are not exposed to a solvent), for example but not limited to, one can use the following conservative substitutions: where Y is substituted with F, T with A or S, I with L or V, W with Y, M with L, N with D, G with A, T with A or S, D with N, I with L or V, F with Y or L, S with A or T and A with S, G, T or V. In some embodiments, non-conservative amino acid substitutions are also encompassed within the term of variants.

The invention includes methods of producing antibodies containing VH and/or VL domain variants of the antibody VH and/or VL domains shown in Table A. Such antibodies may be produced by a method comprising
(i) providing, by way of addition, deletion, substitution or insertion of one or more amino acids in the amino acid sequence of a parent antibody VH domain, an antibody VH domain that is an amino acid sequence variant of the parent antibody VH domain,
   wherein the parent antibody VH domain is the VH domain of QUEL-0101, QUEL-0201 or QUEL-0301 or a VH domain comprising the heavy chain complementarity determining regions of any of those antibodies,
(ii) optionally combining the VH domain thus provided with a VL domain, to provide a VH/VL combination, and
(iii) testing the VH domain or VH/VL domain combination thus provided to identify an antibody with one or more desired characteristics.

The VH domain may be the VH domain of QUEL-0201.

Desired characteristics include binding to human and/or non-human GFRAL. Antibodies with comparable or higher affinity for human and/or mouse GFRAL relative to the parent antibody may be identified. Other desired characteristics include inhibition of GDF15 signalling assays described herein, e.g., ERK phosphorylation assay. Identifying an antibody with a desired characteristic may comprise identifying an antibody with a functional attribute described herein, such as its affinity, cross-reactivity, specificity, or neutralising potency, any of which may be determined in assays as described herein.

When VL domains are included in the method, the VL domain may be a VL domain of any of QUEL-0101, QUEL-0201 or QUEL-0301, or may be a variant provided by way of addition, deletion, substitution or insertion of one or more amino acids in the amino acid sequence of a parent VL domain, wherein the parent VL domain is the VL domain of any of QUEL-0101, QUEL-0201 or QUEL-0301 or a VL domain comprising the light chain complementarity determining regions of any of those antibodies. The VL domain may be the VL domain of the same antibody as the VH domain. It may be the VL domain of QUEL-0201.

Methods of generating variant antibodies may optionally comprise producing copies of the antibody or VH/VL domain combination. Methods may further comprise expressing the resultant antibody. It is possible to produce nucleotide sequences corresponding to a desired antibody VH and/or VL domain, optionally in one or more expression vectors. Suitable methods of expression, including recombinant expression in host cells, are set out in detail herein.

### Encoding Nucleic Acids and Methods of Production

Isolated nucleic acid may be provided, encoding antibodies according to the present invention. Nucleic acid may be DNA and/or RNA. Genomic DNA, cDNA, mRNA or other RNA, of synthetic origin, or any combination thereof can encode an antibody.

The present invention provides constructs in the form of plasmids, vectors, transcription or expression cassettes which comprise at least one polynucleotide as above. Exemplary nucleotide sequences are included in the sequence listing. Reference to a nucleotide sequence as set out herein encompasses a DNA molecule with the specified sequence, and encompasses a RNA molecule with the specified sequence in which U is substituted for T, unless context requires otherwise.

The present invention also provides a recombinant host cell that comprises one or more nucleic acids encoding the antibody. Methods of producing the encoded antibody may comprise expression from the nucleic acid, e.g., by culturing recombinant host cells containing the nucleic acid. The antibody may thus be obtained, and may be isolated and/or purified using any suitable technique, then used as appropriate. A method of production may comprise formulating the product into a composition including at least one additional component, such as a pharmaceutically acceptable excipient.

Systems for cloning and expression of a polypeptide in a variety of different host cells are well known. Suitable host cells include bacteria, mammalian cells, plant cells, filamentous fungi, yeast and baculovirus systems and transgenic plants and animals.

The expression of antibodies and antibody fragments in prokaryotic cells is well established in the art. A common bacterial host is E. coli. Expression in eukaryotic cells in culture is also available to those skilled in the art as an option for production. Mammalian cell lines available in the art for expression of a heterologous polypeptide include Chinese hamster ovary (CHO) cells, HeLa cells, baby hamster kidney cells, NSO mouse melanoma cells, YB2/0 rat myeloma cells, human embryonic kidney cells (e.g., HEK293), human embryonic retina cells and many others.

Vectors may contain appropriate regulatory sequences, including promoter sequences, terminator sequences, polyadenylation sequences, enhancer sequences, marker genes and other sequences as appropriate. Nucleic acid encoding an antibody can be introduced into a host cell. Nucleic acid of the invention may be integrated into the genome (e.g. chromosome) of the host cell. Integration may be promoted by inclusion of sequences that promote recombination with the genome, in accordance with standard techniques. Nucleic acid can be introduced to eukaryotic cells by various methods, including calcium phosphate transfection, DEAE-Dextran, electroporation, liposome-mediated transfection and transduction using retrovirus or other virus, e.g. vaccinia or, for insect cells, baculovirus. Introducing nucleic acid in the host cell, in particular a eukaryotic cell may use a viral or a plasmid based system. The plasmid system may be maintained episomally or may be incorporated into the host cell or into an artificial chromosome. Incorporation may be either by random or targeted integration of one or more copies at single or multiple loci. For bacterial cells, suitable techniques include calcium chloride transformation, electroporation and transfection using bacteriophage. The introduction may be followed by expressing the nucleic acid, e.g., by culturing host cells under conditions for expression of the gene, then optionally isolating or purifying the binder polypeptide, e.g., antibody.

### Formulation and Administration

Compositions are provided comprising inhibitors of GDF15 signalling, e.g., anti-GFRAL antibodies, according to the present invention. Compositions are also provided comprising nucleic acid encoding inhibitors of GDF15 signalling that are binder polypeptides, e.g., antibodies. Such compositions may be provided for use in treatment of the human or animal body by therapy, including in any of the example medical treatments described herein. The compositions may further comprise, in addition to the active ingredient (inhibitor or encoding nucleic acid), one or more pharmaceutically acceptable excipients.

Binder polypeptides according to the present invention, and their encoding nucleic acid molecules, will usually be provided in isolated form. VH and/or VL domains, and nucleic acids may be provided purified from their natural environment or their production environment. Isolated binder polypeptides and isolated nucleic acid will be free or substantially free of material with which they are naturally associated, such as other polypeptides or nucleic acids with which they are found in vivo, or the environment in which they are prepared (e.g., cell culture) when such preparation is by recombinant DNA technology in vitro. Optionally an isolated binder polypeptide or nucleic acid (1) is free of at least some other proteins with which it would normally be found, (2) is essentially free of other proteins from the same source, e.g., from the same species, (3) is expressed by a cell from a different species, (4) has been separated from at least about 50 percent of polynucleotides, lipids, carbohydrates, or other materials with which it is associated in nature, (5) is operably associated (by covalent or noncovalent interaction) with a polypeptide with which it is not associated in nature, or (6) does not occur in nature.

Binder polypeptides or their encoding nucleic acids may be formulated with diluents or adjuvants and still for practical purposes be isolated—for example they may be mixed with carriers if used to coat microtitre plates for use in immunoassays, and may be mixed with pharmaceutically acceptable carriers or diluents when used in therapy. As described elsewhere herein, other active ingredients may also be included in therapeutic preparations. The binder polypeptide may be glycosylated, either naturally in vivo or by systems of heterologous eukaryotic cells such as CHO cells, or it may be (for example if produced by expression in a prokaryotic cell) unglycosylated. The invention encompasses antibodies having a modified glycosylation pattern.

Typically, an isolated product constitutes at least about 5%, at least about 10%, at least about 25%, or at least about 50% of a given sample. A binder polypeptide may be substantially free from proteins or polypeptides or other contaminants that are found in its natural or production environment that would interfere with its therapeutic, diagnostic, prophylactic, research or other use.

The invention provides therapeutic compositions comprising the binder polypeptides described herein. Therapeutic compositions comprising nucleic acid encoding such binder polypeptides are also provided. Encoding nucleic acids are described in more detail elsewhere herein and include DNA and RNA, e.g., mRNA. In therapeutic methods described herein, use of nucleic acid encoding the binder polypeptide, and/or of cells containing such nucleic acid, may be used as alternatives (or in addition) to compositions comprising the binder polypeptide itself. Cells containing nucleic acid encoding the binder polypeptide, optionally wherein the nucleic acid is stably integrated into the genome, thus represent medicaments for therapeutic use in a patient. Nucleic acid encoding the binder polypeptide may be introduced into human cells derived from the intended patient and modified ex vivo. Administration of cells containing the encoding nucleic acid to the patient provides a reservoir of cells capable of expressing the binder polypeptide, which may provide therapeutic benefit over a longer term compared with administration of isolated nucleic acid or the isolated binder polypeptide. Nucleic acid may also be administered directly to the patient for gene therapy. Thus, nucleic acid encoding the binder polypeptide may be provided for use in gene therapy, comprising introducing the encoding nucleic acid into cells of the patient in vivo, so that the nucleic acid is expressed in the patient's cells and provides a therapeutic effect, examples of which are disclosed herein.

Compositions may contain suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTINT™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311. Compositions may comprise the antibody or nucleic acid in combination with medical injection buffer and/or with adjuvant.

Binder polypeptides, or their encoding nucleic acids, may be formulated for the desired route of administration to a patient, e.g., in liquid (optionally aqueous solution) for injection. The composition may optionally be formulated for intravenous or subcutaneous injection.

Various delivery systems are known and can be used to administer the pharmaceutical composition of the invention. Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. The antigen-binding molecules are preferably administered by subcutaneous injection. Administration may be self-administration by a patient, e.g., self-injection.

The pharmaceutical composition can be also delivered in a vesicle, in particular a liposome (see Langer (1990) Science 249:1527-1533; Treat et al. (1989) in Liposomes in the Therapy of Infectious Disease and Cancer, Lopez Berestein and Fidler (eds.), Liss, New York, pp. 353-365; Lopez-Berestein, ibid., pp. 317-327; see generally ibid.).

In certain situations, the pharmaceutical composition can be delivered in a controlled release system. In one embodiment, a pump may be used (see Langer, supra; Sefton (1987) CRC Crit. Ref. Biomed. Eng. 14:201). In another embodiment, polymeric materials can be used; see, Medical Applications of Controlled Release, Langer and Wise (eds.), CRC Pres., Boca Raton, Fla. (1974). In yet another embodiment, a controlled release system can be placed in proximity of the composition's target, thus requiring only a fraction of the systemic dose (see, e.g., Goodson, in Medical Applications of Controlled Release, supra, vol. 2, pp. 115-138, 1984).

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared can be filled in an appropriate ampoule. A pharmaceutical composition of the present invention can be delivered subcutaneously or intravenously with a standard needle and syringe. It is envisaged that treatment will not be restricted to use in the clinic. Therefore, subcutaneous injection using a needle-free device is also advantageous. With respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded. Numerous reusable pen and autoinjector delivery devices have applications in the subcutaneous delivery of a pharmaceutical composition of the present invention. Examples include, but certainly are not limited to AUTOPEN™ (Owen Mumford, Inc., Woodstock, UK), DISETRONIC™ pen (Disetronic Medical Systems, Burghdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and Ill (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPENT™, OPTIPEN PRO™, OPTIPEN STARLET™, and OPTICLIKT™ (Sanofi-Aventis, Frankfurt, Germany), to name only a few. Examples of disposable pen delivery devices having applications in subcutaneous delivery of a pharmaceutical composition of the present invention include, but certainly are not limited to the SOLOSTAR™ pen (Sanofi-Aventis), the FLEXPEN™ (Novo Nordisk), and the KWIKPEN™ (Eli Lilly).

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 5 to about 500 mg per dosage form in a unit dose; especially in the form of injection, the aforesaid antibody may be contained in about 5 to about 100 mg and in about 10 to about 250 mg for the other dosage forms.

The binder polypeptide, nucleic acid, or composition comprising it, may be contained in a medical container such as a phial, syringe, IV container or an injection device. In an example, the binder polypeptide, nucleic acid or composition is in vitro, and may be in a sterile container. In an example, a kit is provided comprising the binder polypeptide, packaging and instructions for use in a therapeutic method as described herein.

One aspect of the invention is a composition comprising a binder polypeptide or nucleic acid of the invention and one or more pharmaceutically acceptable excipients, examples of which are listed above. “Pharmaceutically acceptable” refers to approved or approvable by a regulatory agency of the USA Federal or a state government or listed in the U.S. Pharmacopeia or other generally recognized pharmacopeia for use in animals, including humans. A pharmaceutically acceptable carrier, excipient, or adjuvant can be administered to a patient, together with a binder polypeptide, e.g., any antibody or polypeptide molecule described herein, and does not destroy the pharmacological activity thereof and is nontoxic when administered in doses sufficient to deliver a therapeutic amount of the agent.

In some embodiments, the binder polypeptide will be the sole active ingredient in a composition according to the present invention. Thus, a composition may consist of the antibody or it may consist of the binder polypeptide with one or more pharmaceutically acceptable excipients. However, compositions according to the present invention optionally include one or more additional active ingredients. Other therapeutic agents that it may be desirable to administer with binder polypeptides or nucleic acids according to the present invention include other therapeutic agents for cancer, examples of which are described herein. Any such agent or combination of agents may be administered in combination with, or provided in compositions with binder polypeptides or nucleic acids according to the present invention, whether as a combined or separate preparation. The binder polypeptide or nucleic acid according to the present invention may be administered separately and sequentially, or concurrently and optionally as a combined preparation, with another therapeutic agent or agents such as those mentioned herein.

Multiple compositions can be administered separately or simultaneously. Separate administration refers to the two compositions being administered at different times, e.g. at least 10, 20, 30, or 10-60 minutes apart, or 1, 2, 3, 4, 5, 6, 7, 8, 9, 10, 12 hours apart. One can also administer compositions at 24 hours apart, or even longer apart. Alternatively, two or more compositions can be administered simultaneously, e.g. less than 10 or less than 5 minutes apart. Compositions administered simultaneously can, in some aspects, be administered as a mixture, with or without similar or different time release mechanism for each of the components.

Binder polypeptides, and their encoding nucleic acids, can be used as therapeutic agents. Patients herein are generally mammals, typically humans. A binder polypeptide or nucleic acid may be administered to a mammal, e.g., by any route of administration mentioned herein. In a preferred embodiment, a binder polypeptide is administered by subcutaneous injection.

Administration is normally in a “therapeutically effective amount”, this being an amount that produces the desired effect for which it is administered, sufficient to show benefit to a patient. The exact amount will depend on the purpose of the treatment, and will be ascertainable by one skilled in the art using known techniques (see, for example, Lloyd (1999) The Art, Science and Technology of Pharmaceutical Compounding). Prescription of treatment, e.g. decisions on dosage etc, is within the responsibility of general practitioners and other medical doctors and may depend on the severity of the symptoms and/or progression of a disease being treated. A therapeutically effective amount or suitable dose of binder polypeptide or nucleic acid can be determined by comparing its in vitro activity and in vivo activity in an animal model. Methods for extrapolation of effective dosages in mice and other test animals to humans are known.

In methods of treatment described herein, one or more doses may be administered. In some cases, a single dose may be effective to achieve a long-term benefit. Thus, the method may comprise administering a single dose of the binder polypeptide, its encoding nucleic acid, or the composition. Alternatively, multiple doses may be administered, usually sequentially and separated by a period of days, weeks or months. For example, administration may be every 2 weeks, every 3 weeks or every 4 weeks. Optionally, the binder polypeptide may be administered to a patient once a month, or less frequently, e.g., every two months or every three months.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilised (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment). For treatment to be effective a complete cure is not contemplated. The method can in certain aspects include cure as well. In the context of the invention, treatment may be preventative treatment.

Long half-life is a desirable feature in the binder polypeptides of the present invention. Extended half-life translates to less frequent administration, with fewer injections being required to maintain a therapeutically effective concentration of the molecule in the bloodstream. The in vivo half life of antigen-binding molecules of the present invention in humans may be 7, 8, 9, 10, 11, 12, 13, 14, 15, 16, 17, 18, 19, 20 or 21 days, or longer. The in vivo half life of antigen-binding molecules in non-human primates such as cynomolgus monkeys may be 7, 8, 9, 10, 11, 12, 13, 14, 15, 16, 17, 18, 19, 20 or 21 days, or longer.

Binder polypeptides may be provided for administration at regular intervals of one week, two weeks, three weeks, four weeks, or one month.

### Clauses

The following numbered clauses represent embodiments of the invention and are part of the description.

1. An antibody that binds human GFRAL, comprising an antibody heavy chain variable (VH) domain and an antibody light chain variable (VL) domain,
the VH domain comprising the QUEL-0201 set of heavy chain complementarity determining regions (HCDRs) HCDR1 SEQ ID NO: 13, HCDR2 SEQ ID NO: 14 and HCDR3 SEQ ID NO: 15, and
the VL domain comprising the QUEL-0201 set of light chain complementarity determining regions (LCDRs) LCDR1 SEQ ID NO: 18, LCDR2 SEQ ID NO: 19 and LCDR3 SEQ ID NO: 20.

2. An antibody that binds human GFRAL, comprising
a VH domain having at least 90% amino acid sequence identity to the QUEL-0201 VH domain SEQ ID NO: 12 and
a VL domain having at least 90% amino acid sequence identity to the QUEL-0201 VL domain SEQ ID NO: 17.

3. An antibody that binds human GFRAL comprising
a VH domain encoded by a nucleotide sequence produced by recombination of gene segments IGHV1-3 (e.g., IGHV1-3*01) and IGHJ6 (e.g., IGHJ6*02), and
a VL domain encoded by a nucleotide sequence produced by recombination of gene segments IGLV1-40 (e.g., IGLV1-40*01) and IGLJ3 (e.g., IGLJ3*02).

4. An antibody according to clause 3, wherein the VH domain is encoded by a nucleotide sequence produced by recombination of gene segments IGHV1-3 (e.g., IGHV1-3*01), IGHD5-18 (e.g., IGHD5-18*01) and IGHJ6 (e.g., IGHJ6*02).

5. An antibody that competes for binding human GFRAL with a QUEL-0201 IgG comprising QUEL-0201 VH domain SEQ ID NO: 12 and QUEL-0201 VL domain SEQ ID NO: 17.

6. An antibody according to any of clauses 1 to 5 which binds human GFRAL with an affinity of 1 nM or stronger as determined by surface plasmon resonance.

7. An antibody according to clause 6 which binds human GFRAL with an affinity of 100 pM or stronger as determined by surface plasmon resonance.

8. An antibody according to clause 6 which binds human GFRAL with an affinity in the range 50 pM-200 pM.

9. An antibody according to any of clauses 1 to 8 which cross-reacts with mouse GFRAL, having an affinity for mouse GFRAL within 10-fold of its affinity for human GFRAL.

10. An antibody according to any of clauses 1 to 9 which inhibits GFRAL with a potency of 15 nM or stronger, wherein the potency is determined as IC50 in an in vitro assay of ERK phosphorylation in response to GDF15.

11. An antibody according to clause 10, wherein the potency is 10 nM or stronger.

12. An antibody according to any of clauses 1 to 11, comprising
a VH domain having at least 95% amino acid sequence identity to the QUEL-0201 VH domain SEQ ID NO: 12 and
a VL domain having at least 95% amino acid sequence identity to the QUEL-0201 VL domain SEQ ID NO: 17.

13. An antibody according to any preceding clause, comprising
a VH domain having at least 98% amino acid sequence identity to the QUEL-0201 VH domain SEQ ID NO: 12 and
a VL domain having at least 98% amino acid sequence identity to the QUEL-0201 VL domain SEQ ID NO: 17.

14. An antibody according to clause 13, comprising
the QUEL-0201 VH domain SEQ ID NO: 12, optionally with one or two amino acid alterations and
the QUEL-0201 VL domain SEQ ID NO: 17, optionally with one or two amino acid alterations.

15. An antibody according to clause 14, comprising
the QUEL-0201 VH domain SEQ ID NO: 12 comprising the QUEL-0201 set of HCDRs HCDR1 SEQ ID NO: 13, HCDR2 SEQ ID NO: 14 and HCDR3 SEQ ID NO: 15, optionally with one or two amino acid alterations in the VH domain framework, and
the QUEL-0201 VL domain SEQ ID NO: 17 comprising the QUEL-0201 set of LCDRs LCDR1 SEQ ID NO: 18, LCDR2 SEQ ID NO: 19 and LCDR3 SEQ ID NO: 20, optionally with one or two amino acid alterations in the VL domain framework.

16. An antibody according to clause 14 or clause 15, wherein the one or two amino acid alterations are conservative substitutions.

17. An antibody according to any preceding clause, comprising the QUEL-0201 VH domain SEQ ID NO: 12 and the QUEL-0201 VL domain SEQ ID NO: 17.

18. An antibody that binds human GFRAL, comprising an antibody heavy chain variable (VH) domain and an antibody light chain variable (VL) domain,
the VH domain comprising a set of heavy chain complementarity determining regions (HCDRs), wherein HCDR1 is SEQ ID NO: 23 or SEQ ID NO: 133, HCDR2 is SEQ ID NO: 24, SEQ ID NO: 124, SEQ ID NO: 134 or SEQ ID NO: 142, and HCDR3 is SEQ ID NO: 25, SEQ ID NO: 125, SEQ ID NO: 135 or SEQ ID NO: 143, and
the VL domain comprising a set of light chain complementarity determining regions (LCDRs), wherein LCDR1 is SEQ ID NO: 28, SEQ ID NO: 128 or SEQ ID NO: 138, LCDR2 is SEQ ID NO: 29 or SEQ ID NO: 129 and LCDR3 is SEQ ID NO: 30, SEQ ID NO: 130, SEQ ID NO: 139 or SEQ ID NO: 146.

19. An antibody that binds human GFRAL, comprising an antibody heavy chain variable (VH) domain and an antibody light chain variable (VL) domain,
the VH domain comprising the QUEL-0301 set of heavy chain complementarity determining regions (HCDRs) HCDR1 SEQ ID NO: 23, HCDR2 SEQ ID NO: 24 and HCDR3 SEQ ID NO: 25, and
the VL domain comprising the QUEL-0301 set of light chain complementarity determining regions (LCDRs) LCDR1 SEQ ID NO: 28, LCDR2 SEQ ID NO: 29 and LCDR3 SEQ ID NO: 30.

20. An antibody that binds human GFRAL, comprising a VH domain and a VL domain,
the VH domain comprising the QUEL-0302 set of HCDRs HCDR1 SEQ ID NO: 23, HCDR2 SEQ ID NO: 124 and HCDR3 SEQ ID NO: 125, and
the VL domain comprising the QUEL-0302 set of LCDRs LCDR1 SEQ ID NO: 128, LCDR2 SEQ ID NO: 129 and LCDR3 SEQ ID NO: 130.

21. An antibody that binds human GFRAL, comprising a VH domain and a VL domain,
the VH domain comprising the QUEL-0303 set of HCDRs HCDR1 SEQ ID NO: 133, HCDR2 SEQ ID NO: 134 and HCDR3 SEQ ID NO: 135, and
the VL domain comprising the QUEL-0303 set of LCDRs LCDR1 SEQ ID NO: 138, LCDR2 SEQ ID NO: 29 and LCDR3 SEQ ID NO: 139.

22. An antibody that binds human GFRAL, comprising a VH domain and a VL domain,
the VH domain comprising the QUEL-0304 set of HCDRs HCDR1 SEQ ID NO: 133, HCDR2 SEQ ID NO: 142 and HCDR3 SEQ ID NO: 143, and
the VL domain comprising the QUEL-0304 set of LCDRs LCDR1 SEQ ID NO: 138, LCDR2 SEQ ID NO: 29 and LCDR3 SEQ ID NO: 146.

23. An antibody that binds human GFRAL, comprising
a VH domain having at least 90% amino acid sequence identity to the QUEL-0301 VH domain SEQ ID NO: 22 and
   a VL domain having at least 90% amino acid sequence identity to the QUEL-0301 VL domain SEQ ID NO: 27.

24. An antibody that binds human GFRAL comprising
a VH domain encoded by a nucleotide sequence produced by recombination of gene segments IGHV3-7 (e.g., IGHV3-7*01) and IGHJ4 (e.g., IGHJ4*02), and
a VL domain encoded by a nucleotide sequence produced by recombination of gene segments IGLV1-44 (e.g., IGLV1-44*01) and IGLJ3 (e.g., IGLJ3*02).

25. An antibody according to clause 24, wherein the VH domain is encoded by a nucleotide sequence produced by recombination of gene segments IGHV3-7 (e.g., IGHV3-7*01), IGHD1-7 (e.g., IGHD1-7*01) and IGHJ4 (e.g., IGHJ4*02).

26. An antibody that binds human GFRAL comprising
a VH domain encoded by a nucleotide sequence produced by recombination of gene segments IGHV3-7 (e.g., IGHV3-7*01) and IGHJ4 (e.g., IGHJ4*02), and
a VL domain encoded by a nucleotide sequence produced by recombination of gene segments IGLV1-47 (e.g., IGLV1-47*01) and IGLJ3 (e.g., IGLJ3*02).

27. An antibody according to clause 26, wherein the VH domain is encoded by a nucleotide sequence produced by recombination of gene segments IGHV3-7 (e.g., IGHV3-7*01), IGHD1-20 (e.g., IGHD1-20*01) and IGHJ4 (e.g., IGHJ4*02).

28. An antibody that competes for binding human GFRAL with a QUEL-0301 IgG comprising QUEL-0301 VH domain SEQ ID NO: 22 and QUEL-0301 VL domain SEQ ID NO: 27.

29. An antibody according to any of clauses 18 to 28 which binds human GFRAL with an affinity of 1 nM or stronger as determined by surface plasmon resonance.

30. An antibody according to clause 29 which binds human GFRAL with an affinity of 1 pM or stronger as determined by surface plasmon resonance.

31. An antibody according to clause 29 which binds human GFRAL with an affinity in the range 0.5 pM-2 pM.

32. An antibody according to any of clauses 18 to 31 which cross-reacts with mouse GFRAL, having an affinity for mouse GFRAL of 10 nM or stronger as determined by surface plasmon resonance.

33. An antibody according to any of clauses 18 to 32 which inhibits GFRAL with a potency of 15 nM or stronger, wherein the potency is determined as IC50 in an in vitro assay of ERK phosphorylation in response to GDF15.

34. An antibody according to clause 33, wherein the potency is 10 nM or stronger.

35. An antibody according to any of clauses 18 to 34, comprising
a VH domain having at least 95% amino acid sequence identity to the QUEL-0301 VH domain SEQ ID NO: 22 and
a VL domain having at least 95% amino acid sequence identity to the QUEL-0301 VL domain SEQ ID NO: 27.

36. An antibody according to any preceding clause, comprising
a VH domain having at least 98% amino acid sequence identity to the QUEL-0301 VH domain SEQ ID NO: 22 and
a VL domain having at least 98% amino acid sequence identity to the QUEL-0301 VL domain SEQ ID NO: 27.

37. An antibody according to clause 36, comprising
the QUEL-0301 VH domain SEQ ID NO: 22, optionally with one or two amino acid alterations and
the QUEL-0301 VL domain SEQ ID NO: 27, optionally with one or two amino acid alterations.

38. An antibody according to clause 37, comprising
the QUEL-0301 VH domain SEQ ID NO: 22 comprising the QUEL-0301 set of HCDRs HCDR1 SEQ ID NO: 23, HCDR2 SEQ ID NO: 24 and HCDR3 SEQ ID NO: 25, optionally with one or two amino acid alterations in the VH domain framework, and
the QUEL-0301 VL domain SEQ ID NO: 27 comprising the QUEL-0301 set of LCDRs LCDR1 SEQ ID NO: 28, LCDR2 SEQ ID NO: 29 and LCDR3 SEQ ID NO: 30, optionally with one or two amino acid alterations in the VL domain framework.

39. An antibody according to clause 37 or clause 38, wherein the one or two amino acid alterations are conservative substitutions.

40. An antibody according to any of clauses 18 to 39, comprising the QUEL-0301 VH domain SEQ ID NO: 22 and the QUEL-0301 VL domain SEQ ID NO: 27.

41. An antibody according to any of clauses 18, 20 and 23-28, comprising the QUEL-0302 VH domain SEQ ID NO: 123 and the QUEL-0302 VL domain SEQ ID NO: 127.

42. An antibody according to any of clauses 18, 21 and 23-28, comprising the QUEL-0303 VH domain SEQ ID NO: 132 and the QUEL-0303 VL domain SEQ ID NO: 137.

43. An antibody according to any of clauses 18 and 22-28, comprising the QUEL-0304 VH domain SEQ ID NO: 141 and the QUEL-0304 VL domain SEQ ID NO: 145.

44. An antibody that binds human GFRAL, comprising an antibody heavy chain variable (VH) domain and an antibody light chain variable (VL) domain,
the VH domain comprising a set of HCDRs HCDR1, HCDR2 and HCDR3, wherein
HCDR1 is SEQ ID NO: 3, SEQ ID NO: 99, SEQ ID NO: 107 or SEQ ID NO: 118,
HCDR2 is SEQ ID NO: 4, SEQ ID NO: 100 or SEQ ID NO: 108 and
HCDR3 is SEQ ID NO: 5, SEQ ID NO: 101, SEQ ID NO: 113 or SEQ ID NO: 119, and
the VL domain comprising a set of LCDRs LCDR1, LCDR2 and LCDR3, wherein
LCDR1 is SEQ ID NO: 8,
LCDR2 is SEQ ID NO: 9 and
LCDR3 is SEQ ID NO: 10 or SEQ ID NO: 104.

45. An antibody that binds human GFRAL, comprising an antibody heavy chain variable (VH) domain and an antibody light chain variable (VL) domain,
the VH domain comprising the QUEL-0101 set of heavy chain complementarity determining regions (HCDRs) HCDR1 SEQ ID NO: 3, HCDR2 SEQ ID NO: 4 and HCDR3 SEQ ID NO: 5, and
the VL domain comprising the QUEL-0101 set of light chain complementarity determining regions (LCDRs) LCDR1 SEQ ID NO: 8, LCDR2 SEQ ID NO: 9 and LCDR3 SEQ ID NO: 10.

46. An antibody that binds human GFRAL, comprising a VH domain and a VL domain,
the VH domain comprising the QUEL-0102 set of HCDRs HCDR1 SEQ ID NO: 99, HCDR2 SEQ ID NO: 100 and HCDR3 SEQ ID NO: 101, and the VL domain comprising the QUEL-0102 set of LCDRs LCDR1 SEQ ID NO: 8, LCDR2 SEQ ID NO: 9 and LCDR3 SEQ ID NO: 104.

47. An antibody that binds human GFRAL, comprising a VH domain and a VL domain,
the VH domain comprising the QUEL-0103 set of HCDRs HCDR1 SEQ ID NO: 107, HCDR2 SEQ ID NO: 108 and HCDR3 SEQ ID NO: 5, and
the VL domain comprising the QUEL-0103 set of LCDRs LCDR1 SEQ ID NO: 8, LCDR2 SEQ ID NO: 9 and LCDR3 SEQ ID NO: 104.

48. An antibody that binds human GFRAL, comprising a VH domain and a VL domain,
the VH domain comprising the QUEL-0104 set of HCDRs HCDR1 SEQ ID NO: 107, HCDR2 SEQ ID NO: 108 and HCDR3 SEQ ID NO: 113, and
the VL domain comprising the QUEL-0104 set of LCDRs LCDR1 SEQ ID NO: 8, LCDR2 SEQ ID NO: 9 and LCDR3 SEQ ID NO: 104.

49. An antibody that binds human GFRAL, comprising a VH domain and a VL domain,
the VH domain comprising the QUEL-0105 set of HCDRs HCDR1 SEQ ID NO: 118, HCDR2 SEQ ID NO: 108 and HCDR3 SEQ ID NO: 119, and
the VL domain comprising the QUEL-0105 set of LCDRs LCDR1 SEQ ID NO: 8, LCDR2 SEQ ID NO: 9 and LCDR3 SEQ ID NO: 104.

50. An antibody that binds human GFRAL, comprising
a VH domain having at least 90% amino acid sequence identity to the QUEL-0101 VH domain SEQ ID NO: 2 and
a VL domain having at least 90% amino acid sequence identity to the QUEL-0101 VL domain SEQ ID NO: 7.

51. An antibody that binds human GFRAL comprising
a VH domain encoded by a nucleotide sequence produced by recombination of gene segments IGHV3-30 (e.g., IGHV3-30*18) and IGHJ6 (e.g., IGHJ6*02), and
a VL domain encoded by a nucleotide sequence produced by recombination of gene segments IGKV1-27 (e.g., IGKV1-27*01) and IGKJ4 (e.g., IGKJ4*01).

52. An antibody according to clause 51, wherein the VH domain is encoded by a nucleotide sequence produced by recombination of gene segments IGHV3-30*18 (e.g., IGHV3-30), IGHD3-10 (e.g., IGHD3-10*01) and IGHJ6 (e.g., IGHJ6*02).

53. An antibody that competes for binding human GFRAL with a QUEL-0101 IgG comprising QUEL-0101 VH domain SEQ ID NO: 2 and QUEL-0101 VL domain SEQ ID NO: 7.

54. An antibody according to any of clauses 44 to 53 which binds human GFRAL with an affinity of 1 nM or stronger as determined by surface plasmon resonance.

55. An antibody according to clause 54 which binds human GFRAL with an affinity of 200 pM or stronger as determined by surface plasmon resonance.

56. An antibody according to clause 54 which binds human GFRAL with an affinity in the range 100 pM-400 pM.

57. An antibody according to any of clauses 44 to 56 which cross-reacts with mouse GFRAL, having an affinity for mouse GFRAL within 10-fold of its affinity for human GFRAL.

58. An antibody according to any of clauses 44 to 57 which inhibits GFRAL with a potency of 15 nM or stronger, wherein the potency is determined as IC50 in an in vitro assay of ERK phosphorylation in response to GDF15.

59. An antibody according to clause 58, wherein the potency is 10 nM or stronger.

60. An antibody according to clause 59, wherein the potency is 5 nM or stronger.

61. An antibody according to any of clauses 44 to 60, comprising
a VH domain having at least 95% amino acid sequence identity to the QUEL-0101 VH domain SEQ ID NO: 2 and
a VL domain having at least 95% amino acid sequence identity to the QUEL-0101 VL domain SEQ ID NO: 7.

62. An antibody according to any of clauses 44 to 61, comprising
a VH domain having at least 98% amino acid sequence identity to the QUEL-0101 VH domain SEQ ID NO: 2 and
a VL domain having at least 98% amino acid sequence identity to the QUEL-0101 VL domain SEQ ID NO: 7.

63. An antibody according to clause 62, comprising
the QUEL-0101 VH domain SEQ ID NO: 2, optionally with one or two amino acid alterations and
the QUEL-0101 VL domain SEQ ID NO: 7, optionally with one or two amino acid alterations.

64. An antibody according to clause 63, comprising
the QUEL-0101 VH domain SEQ ID NO: 2 comprising the QUEL-0101 set of HCDRs HCDR1 SEQ ID NO: 3, HCDR2 SEQ ID NO: 4 and HCDR3 SEQ ID NO: 5, optionally with one or two amino acid alterations in the VH domain framework, and
the QUEL-0101 VL domain SEQ ID NO: 7 comprising the QUEL-0101 set of LCDRs LCDR1 SEQ ID NO: 8, LCDR2 SEQ ID NO: 9 and LCDR3 SEQ ID NO: 10, optionally with one or two amino acid alterations in the VL domain framework.

65. An antibody according to clause 63 or clause 64, wherein the one or two amino acid alterations are conservative substitutions.

66. An antibody according to any of clauses 44 to 65, comprising the QUEL-0101 VH domain SEQ ID NO: 2 and the QUEL-0101 VL domain SEQ ID NO: 7.

67. An antibody according to any of clauses 44, 46 and 50-53, comprising the QUEL-0102 VH domain SEQ ID NO: 98 and the QUEL-0102 VL domain SEQ ID NO: 103.

68. An antibody according to any of clauses 44, 47 and 50-53, comprising the QUEL-0103 VH domain SEQ ID NO: 106 and the QUEL-0103 VH domain SEQ ID NO: 110.

69. An antibody according to any of clauses 44, 48 and 50-53, comprising the QUEL-0104 VH domain SEQ ID NO: 112 and the QUEL-0104 VL domain SEQ ID NO: 115.

70. An antibody according to any of clauses 44 and 49-53, comprising the QUEL-0105 VH domain SEQ ID NO: 117 and the QUEL-0105 VH domain SEQ ID NO: 121.

71. An antibody according to any preceding clause, comprising a heavy chain Fc region.

72. An antibody according to clause 71, which is a human IgG.

73. An antibody according to clause 72, which is a human IgG4.

74. An antibody according to clause 73 comprising heavy chain constant region SEQ ID NO: 60.

75. Nucleic acid encoding an antibody as defined in any preceding clause.

76. A host cell in vitro comprising nucleic acid as defined in clause 75.

77. A composition comprising an antibody according to any of clauses 1 to 74 or nucleic acid according to clause 75, formulated with a pharmaceutically acceptable excipient.

78. A composition according to clause 77, formulated for intravenous or subcutaneous injection.

79. A method of treating a medical condition associated with the GDF15-GFRAL pathway in a patient, comprising administering a composition according to clause 77 or clause 78 to the patient.

80. A composition according to clause 77 or clause 78 for use in treating a medical condition associated with the GDF15-GFRAL pathway in a patient.

81. Use of a composition according to clause 77 or clause 78 for the manufacture of a medicament for treating a medical condition associated with the GDF15-GFRAL pathway in a patient.

82. A method according to clause 79, a composition according to clause 80 or use of a composition according to clause 81, wherein the medical condition is hyperemesis gravidarum, anorexia, cachexia, conditioned taste aversion and/or a side effect of chemotherapy treatment for cancer.

83. A method, composition or use according to clause 82, wherein the patient is also to receive, or has received, treatment with an anti-cancer chemotherapeutic agent.

84. A method, composition or use according to clause 82 or clause 83, wherein the medical condition is anorexia in a cancer patient.

85. A method, composition or use according to clause 82 or clause 83, wherein the medical condition is cachexia in a cancer patient.

86. A method, composition or use according to any of clauses 82 to 85, wherein the medical condition is a side effect of treatment with an anti-cancer chemotherapeutic agent.

87. A method, composition or use according to clause 86, wherein the treatment comprises preventing conditioned taste aversion caused by treatment with an anti-cancer chemotherapeutic agent.

88. A method, composition or use according to any of clauses 82 to 87, wherein the treatment comprises reducing body weight loss and extending survival in a cancer patient.

89. A method of reducing glucocorticoid (e.g., cortisol) level in a patient, comprising administering an inhibitor of GDF15 signalling to the patient.

90. An inhibitor of GDF15 signalling for use in reducing glucocorticoid (e.g., cortisol) level in a patient.

91. Use of an inhibitor of GDF15 signalling for the manufacture of a medicament for reducing glucocorticoid (e.g., cortisol) level in a patient.

92. A method according to clause 89, an inhibitor for use according to clause 90, or use of an inhibitor according to clause 91, for normalising the blood level of glucocorticoid (e.g., cortisol) in a patient who has been determined to have an elevated blood glucocorticoid level.

93. A method according to clause 89, an inhibitor for use according to clause 90 or use of an inhibitor according to clause 91, wherein the patient is a cancer patient.

94. A method, an inhibitor for use, or use of an inhibitor according to any of clauses 89 to 93, wherein the patient is also to receive, or has received, treatment with a cytotoxic and/or antineoplastic agent.

95. A method, an inhibitor for use, or use of an inhibitor according to any of clauses 89 to 94, wherein the inhibitor inhibits the GDF15-GFRAL-RET signalling complex.

96. A method, an inhibitor for use, or use of an inhibitor according to clause 95, wherein the inhibitor is an anti-GFRAL antibody.

97. A method, an inhibitor for use, or use of an inhibitor according to clause 96, wherein the anti-GFRAL antibody is an antibody as defined in any of clauses 1 to 74.

### EXAMPLES

In these Examples we present the characterisation of anti-GFRAL antagonistic antibodies QUEL-0101 (and its variants QUEL-0102, QUEL-0103, QUEL-0104 and QUEL-0105), QUEL-0201, and QUEL-0301 (and its variants QUEL-0302, QUEL-0303 and QUEL-0304). We demonstrate the ability of anti-GFRAL antagonist antibodies to bind and potently inhibit GFRAL. We show that such blockade of GFRAL inhibits the action of GDF15 on food intake, body weight and markers of muscle atrophy in vivo in mice. We further show that, at the same dose, anti-GFRAL antibody also inhibits the effect of GDF15 acting through GFRAL to increase circulating glucocorticoid levels. This work demonstrates that inhibition of GDF15:GFRAL interaction inhibits a neuroendocrine response to GDF15 comprising elevation of glucocorticoid, and indicates the possibility of using an anti-GFRAL antagonist antibody therapeutically to lower glucocorticoid levels in patients, e.g., to reduce an elevated blood level of glucocorticoid (e.g., cortisol) closer to a normal physiological level in the patient.

### Example 1. In Vitro ERK Phosphorylation Assay for Inhibition of GFRAL Signalling

HEK293 cells were engineered to express DNA encoding human GFRAL SEQ ID NO: 31 and/or human RET on the cell surface. Human GDF15 (recombinant human GDF15 protein with 6-His tag (R&D Systems, 957-GD)) was added to 96-well cell culture containing the following HEK293 cell lines:
HEK293 cells expressing human GFRAL only
HEK293 cells expressing human RET only
HEK293 cells co-expressing human GFRAL and RET.

The cells were incubated with GDF15 4 nM, then medium was removed and cell samples were lysed. Cell lysates were transferred into 384 well plates and an HTRF assay was performed to measure the phosphorylation of ERK gene product, representing the downstream signal of the GDF15-GFRAL-RET pathway. When RET is activated by GDF15-GFRAL tetramer, it triggers phosphorylation of ERK, which is detectable by HTRF. We used the Cisbio Advanced Phospho-ERK assay kit to detect phosphorylated ERK in a sandwich assay format using 2 different antibodies specific for phosphorylated ERK, one labelled with Eu3+-Cryptate (donor) and the second with d2 (acceptor). When the dyes are in close proximity, which occurs when the two antibodies are bound to phosphorylated ERK, the excitation of the donor with a light source triggers a Fluorescence Resonance Energy Transfer (FRET) towards the acceptor, which in turn fluoresces at a specific wavelength (665 nm). Change in fluorescence (ΔF) represents the effect of GDF15 downstream signalling. A plot of ΔF against log of the concentration of GDF15 generates a standard sigmoid curve in cells co-expressing GFRAL and RET, but no signal in cells expressing GFRAL or RET alone (FIG. 3).

As a positive control, anti-GFRAL antagonistic antibody mAb Q was included for comparison.

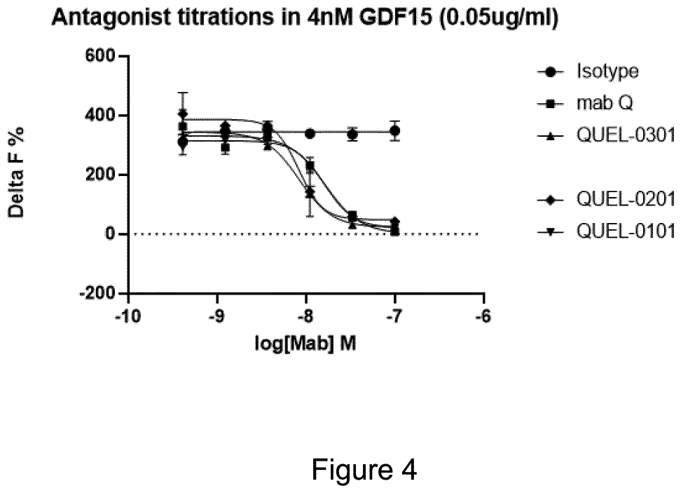

Results for the selected QUEL-0101, QUEL-0202 and QUEL-0301 antibodies and for mAb Q are shown in the table below and in FIG. 4.

| mAb | IC50 | |
|---|---|---|
| QUEL-0101 | 2.8 | nM |
| QUEL-0201 | 8.6 | nM |
| QUEL-0301 | 9.4 | nM |
| mAb Q | 20 | nM |

### Example 2. Affinity for Human GFRAL Determined by SPR

QUEL-0101, QUEL-0201 and QUEL-0301 showed a good range of affinity to human GFRAL as determined by SPR.

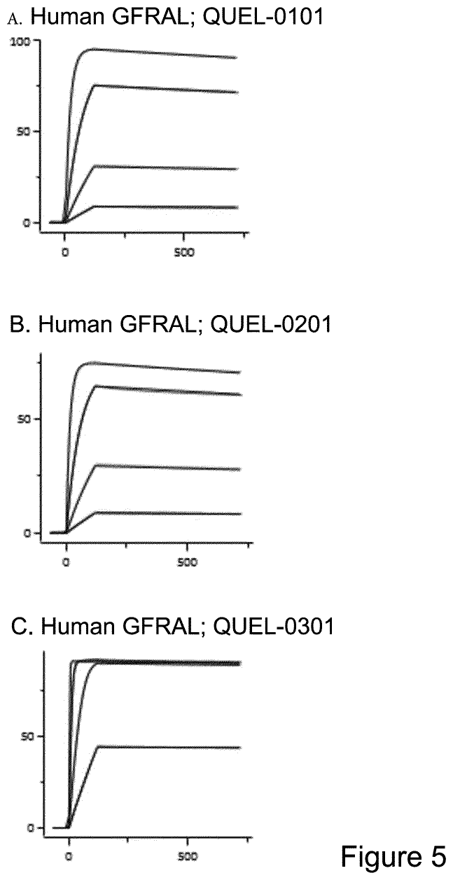

Binding of antibodies to human GFRAL was measured by SPR using His-tagged human GFRAL as analyte, and test antibody coupled to the surface of a CM4 biosensor chip via immobilised anti-human Fc antibody. Running buffer was HBS-P+ buffer pH 7.4 (GE BR100671) with 1 mM CaCl₂. Antibodies were captured for 60 seconds at 1 μg/ml (˜80-140 RU captured, depending on the binder). Analyte (recombinant soluble GFRAL protein, R&D catalogue no. 9647-GR-050) was injected at 100, 25, 6.25, 1.56, 0.39, 0.098, 0.024 and 0 nM for 120 seconds association time at 30 μg/min and dissociation was monitored for 1200 seconds. The chip surface was regenerated using 10 mM glycine pH 1.5. All experiments were performed at 25° C. Sensorgrams for each antibody were double subtracted (reference cell and 0 concentration). Data were fitted to a 1:1 binding interaction model. Sensorgrams were fitted using multiple cycle kinetics mode. Each of QUEL-0101, QUEL-0201 and QUEL-0301 showed a fast on rate and slow dissociation. FIG. 5.

KD and other kinetic constants for binding to human GFRAL were calculated and results are shown in the table below.

| mAb | kₐ (1/Ms) | k_{d} (1/s) | K_{D} (M) |
|---|---|---|---|
| QUEL-0101 | 5.61E+05 | 7.87E−05 | 1.40E−10 |
| QUEL-0201 | 7.38E+05 | 6.19E−05 | 8.39E−11 |
| QUEL-0301 | 9.83E+06 | 9.72E−06 | 9.88E−13 |
| mAb Q | 2.38E+06 | 1.03E−05 | 4.30E−12 |

Of these antibodies, QUEL-0301 had the highest affinity for human GFRAL at approximately 1 picomolar (pM), stronger than the reference antibody mAb Q which had a measured affinity of 4.3 pM. The ˜4-fold difference between these antibodies indicates a comparable affinity (2-fold difference is within the experimental error of the Biacore apparatus). The indicated KD value is merely an estimate due to the very slow dissociation of antibody QUEL-0301, with a kd approaching the measurement limit of the apparatus. QUEL-0201 bound human GFRAL with an affinity of approximately 84 pM. QUEL-0101 bound human GFRAL with an affinity of 140 pM.

The QUEL antibodies have very good kinetic characteristics. Their kinetic constants are in the range that one would aspire to in a potent therapeutic antibody against a receptor. The Koff and Kon values are very informative here, showing that the antibodies bind very fast to GFRAL, and stay attached for a long time.

### Example 3. Affinity for Mouse GFRAL Determined by SPR

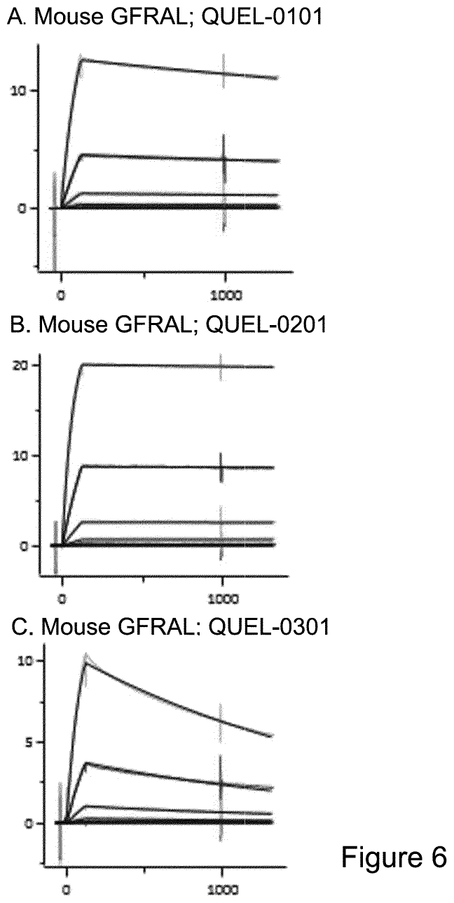

Affinity for mouse GFRAL was determined by SPR by the same method as in Example 2 using a mouse GFRAL-Fc fusion (R&D catalogue no. 9844-GR-050050) as analyte. FIG. 6.

KD and other kinetic constants for binding to mouse GFRAL were calculated and results are shown in the table below.

| mAb | kₐ (1/Ms) | kₐ (1/s) | K_{D} (M) |
|---|---|---|---|
| QUEL-0101 | 9.37E+04 | 1.12E−04 | 1.19E−09 |
| QUEL-0201 | 1.53E+05 | 9.90E−06 | 6.48E−11 |
| QUEL-0301 | 1.06E+05 | 5.24E−04 | 4.95E−09 |
| mAb Q | 2.39E+05 | 4.19E−06 | 1.76E−11 |

Of these antibodies, QUEL-0201 had the highest affinity for mouse GFRAL at approximately 64.8 pM), QUEL-0101 had an affinity of approximately 1.2 nanomolar (nM), and QUEL-0301 had relatively low affinity at just under 5 nM.

Comparing the measured affinities of these antibodies for human and mouse GFRAL, QUEL-0201 has greatest cross-reactivity, having an affinity difference within 1.5-fold for human and mouse GFRAL (human ˜84 pM; mouse ˜65 pM). QUEL-0101 also had good cross-reactivity, within 10-fold, although the absolute affinities were lower (human ˜0.14 nM; mouse ˜1.2 nM). QUEL-0301 was also cross-reactive but showed relatively weak binding to mouse GFRAL in contrast to its high affinity for human GFRAL (human ˜1 pM; mouse ˜5 nM).

### Example 4. Analysis of Antibody Inhibition of GDF15-GFRAL-RET Complex Formation

Epitope mapping assays were performed for selected antagonistic mAbs to investigate possible mechanisms of inhibiting GDF15-GFRAL-RET signalling.

### (a) Sandwich Assay

In this assay, pairs of antibodies are tested for their ability to simultaneously bind antigen. A first antibody is coupled via its Fc region to a solid support and the GFRAL antigen is added in solution, allowing formation of an antibody-antigen complex. A second antibody is then added in solution. If binding of the second antibody is detected, this indicates that the first and second antibody bind different epitopes. If binding of the second antibody is not detected, this indicates that the first and second antibody compete for binding to the same epitope.

The same principle may be used to assess whether an antibody and a native ligand compete for binding to antigen. Thus, a test antibody is coupled to a solid support and the GFRAL antigen is added in solution, allowing formation of an antibody-GFRAL complex. GDF15 is then added in solution. If binding of GDF15 is detected, this indicates that the antibody does not inhibit formation of a GDF15-GFRAL complex.

Here, we used surface plasmon resonance to detect binding to antibody coupled to the surface of a biosensor chip via a pre-immobilised anti-Fc capture antibody. The following results were obtained in the sandwich assay with test antibodies, GFRAL antigen and either GDF15 or mAbQ as analyte:

| | |
|---|---|
| Bound | Analyte |

| antibody | GDF15 | mAbQ |
|---|---|---|
| QUEL-0101 | 87.3 | 305.6 |
| QUEL-0201 | 83.9 | 268.3 |
| QUEL-0301 | 77.3 | 297.5 |
| mAb Q | 80.2 | 4.0 |
| IgG4PE (negative control) | 1.3 | 4.0 |

Results in the table are shown as resonance units after GDF15 or mAbQ injection.

None of QUEL-0101, QUEL-0201, QUEL-0301 and reference antibody mAb Q appear to inhibit binding of GDF15 to GFRAL, since a binding signal was observed following addition of GDF15 to the antibody-GFRAL complex. The observation that none of QUEL-0101, QUEL-0201 and QUEL-0301 compete with GDF15 for binding to GFRAL indicates that these antibodies inhibit GFRAL signalling via a different mechanism, such as inhibiting association of RET with GDF15-GFRAL. Formation of the GDF15-GFRAL-RET active signalling complex is thus inhibited.

Each of QUEL-0101, QUEL-0201 and QUEL-0301 appear to bind a different epitope of GFRAL compared with mAb Q, since a binding signal was observed following addition of mAb Q to the antibody-GFRAL complex.

### (b) Tandem Binding Assay

The tandem assay is an alternative to the sandwich assay described in (a) above. In the tandem assay, it is the antigen rather than the antibody which is surface bound. Human GFRAL with a 6His tag was captured on an anti-His antibody immobilised on the surface of a biosensor chip, and the first antibody was injected over the antigen followed by the second antibody. Using the tandem method, the QUEL antibodies were separated into two epitope bins. QUEL-0201 recognises a different epitope compared to QUEL-0101 and QUEL-0301 which share one epitope bin. None of the QUEL antibodies competed with mAb Q in this assay (confirming the results from the sandwich assay), which thus places mAb Q in a third epitope bin.

### Example 5. In Vivo Activity of QUEL-0201 and QUEL-0301

For use in murine studies, QUEL-0201 and QUEL-0301 were generated as mouse IgG1 isotype, i.e., with a mouse IgG1 heavy chain constant region, while retaining their human variable domains having the sequences as defined herein.

C57BL/6 mice housed at 22° C. received injections of QUEL-0201 or QUEL-0301, followed by injection of GDF15, and their food intake and body weight were monitored. Treatment groups were as follows:
1. 2 injections of 10 mg/kg QUEL-0301 on Thu and Sat
2. 2 injections of 10 mg/kg QUEL-0201 on Thur and Sat
3. 1 injection of 20 mg/kg QUEL-0301 on Sat
4. 1 injection of 20 mg/kg QUEL-0201 on Sat
5. 2 injections of isotype control antibody on Thu and Sat
   All groups received injections of recombinant human GDF15 on Sunday.

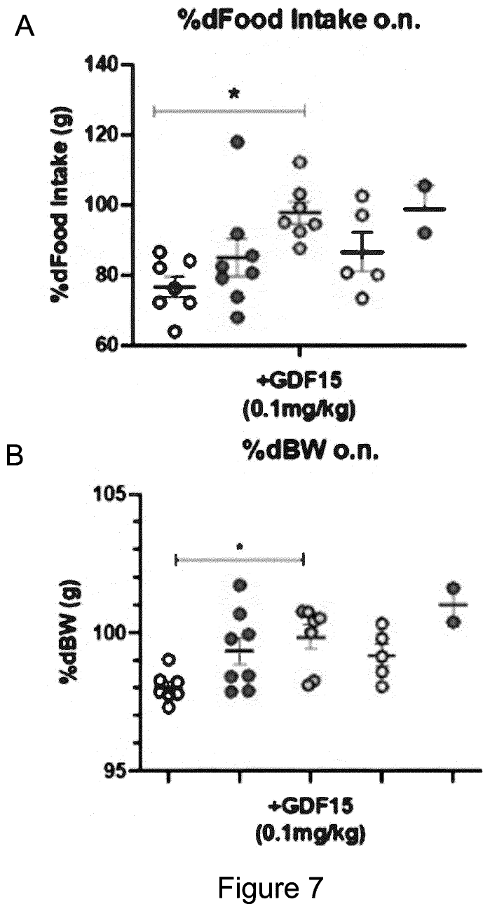

Mice treated with the isotype control antibody reduced their food intake over the study period. Mice treated with anti-GFRAL antibody QUEL-0201 also reduced their food intake, but by less than the isotype control group. Mice treated with anti-GFRAL antibody QUEL-0301 maintained their food intake. FIG. 7 A.

Consistent with the effects on food intake, mice in the control group showed a drop in body weight, while mice treated with QUEL-0301 showed a lesser drop in body weight, and mice treated with QUEL-0201 showed no loss of body weight.

The greater efficacy of QUEL-0201 in this study is attributed to its greater affinity for mouse GFRAL compared with QUEL-0301 (Example 3).

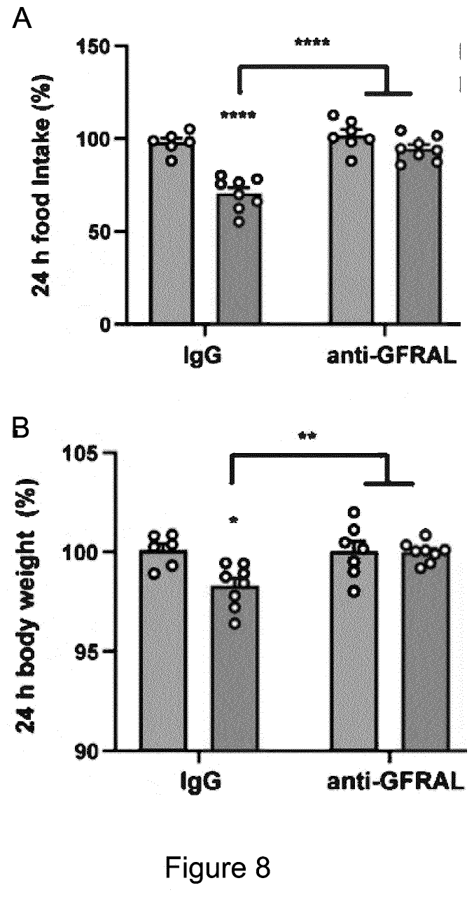

In a further mouse study (MS15) we confirmed that, when administered in two injections each at a dose of 20 mg/kg, QUEL-0201 blocked GDF15-induced food intake reduction and body weight loss (FIGS. 8 A and 8 B).

### Example 6. Exogenous GDF15 Activates the HPA Axis in a GFRAL-Dependent Manner

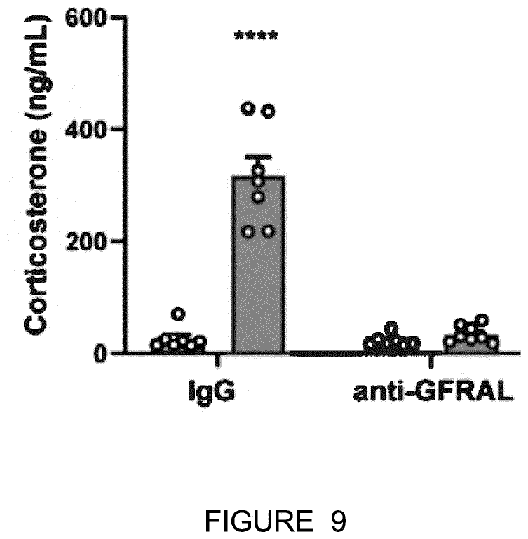

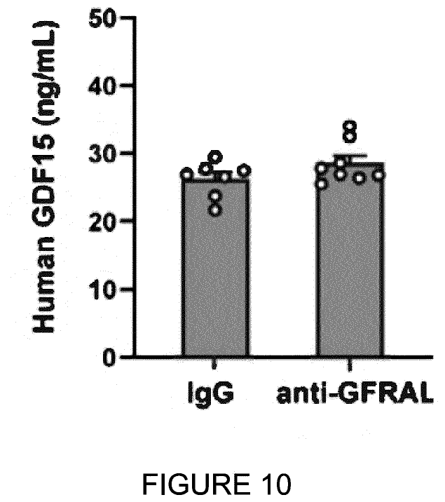

To determine whether GDF15, acting through its hindbrain receptor, might increase corticosterone levels in the mouse, we undertook experiments using anti-GFRAL antibody QUEL-0201, having validated its efficacy on classical GDF15 responses in mice (MS15) (Example 5). QUEL-0201 completely prevented GDF15-induced corticosterone concentrations while a control isotype antibody had no effect (FIG. 9). The concentrations of human circulating GDF15 achieved in the study did not differ between the anti-GFRAL group and control isotype (FIG. 10).

Thus, at the same dose which was effective in fully blocking the effect of GDF15 on food intake and body weight, the anti-GFRAL antibody also fully blocked the corticosterone response to GDF15.

### Example 7. Sequence Variants of QUEL-0101

We isolated antibodies from immunised mice which have sequences that appear to share the same or parallel evolutionary lineage from germline as QUEL-0101, on the basis of their high sequence homology with the QUEL-0101 VH and VL domains. These are designated QUEL-0102, QUEL-0103, QUEL-0104 and QUEL-0105. Each of these clones displayed binding to human GFRAL.

### Example 8. Sequence Variants of QUEL-0301

We isolated antibodies from immunised mice which have sequences that appear to share the same or parallel evolutionary lineage from germline as QUEL-0301, on the basis of their high sequence homology with the QUEL-0301 VH and VL domains. These are designated QUEL-0302, QUEL-0303 and QUEL-0304 respectively. Each of these clones displayed binding to human GFRAL. QUEL-0304 was confirmed to recognise human GFRAL in a further in vitro primary screening assay.

### Example 9. Inhibition of GFRAL Reduces Biomarkers of Muscle Atrophy

We analysed the effect of GDF15 and anti-GFRAL antibody on expression of Mafbx, Murf1 and Foxo1. These genes are increased transcriptionally in skeletal muscle under atrophy-inducing conditions, making them excellent markers of muscle atrophy [.52, .53]. We show here that treatment with anti-GFRAL antibody QUEL-0201 inhibited GDF15-induced increase in each of these markers in vivo in mouse skeletal muscle, thus providing further support for the use of GDF15 inhibitors in treating conditions involving loss of skeletal muscle mass, such as cachexia.

### Materials and Methods

BL6/C57 mice housed at 22° C. received intraperitoneally once daily for 4 days either anti-GFRAL antibody QUEL-0201 (dose of 20 mg/kg) or isotype control. The day after the last injection, mice received either human recombinant GDF15 (0.1 mg/kg, Qkine) or vehicle control via subcutaneous injection. All groups (n=4-5 mice) were sacrificed 6 h later and four muscles were collected: Tibialis anterior, Extensor digitorum longus, Gastrocnemius, Soleus. RNA was extracted, purified, and analysed as previously described [49]. Three well-recognised markers of skeletal muscle atrophy were analysed: Mafbx, Murf1 and Foxo1.

### Results

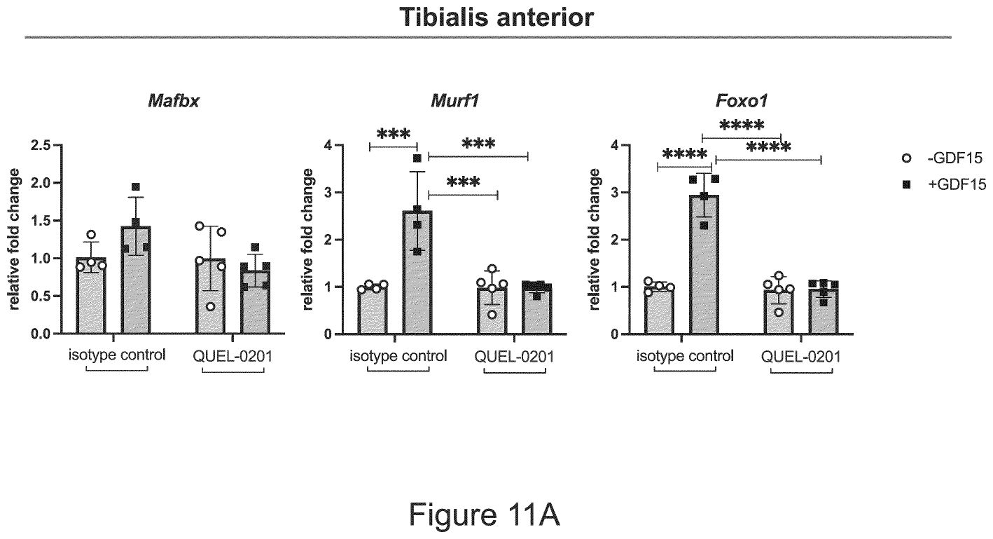

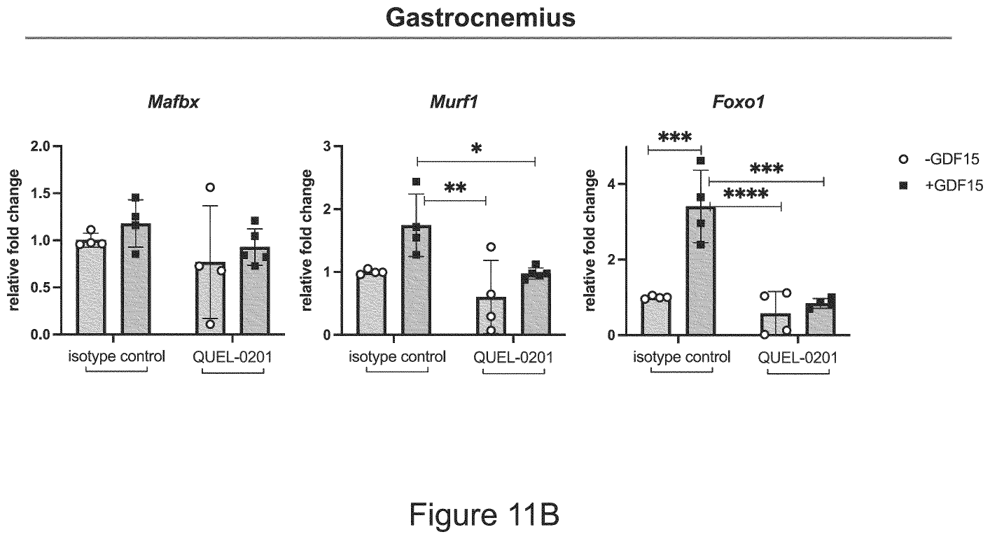

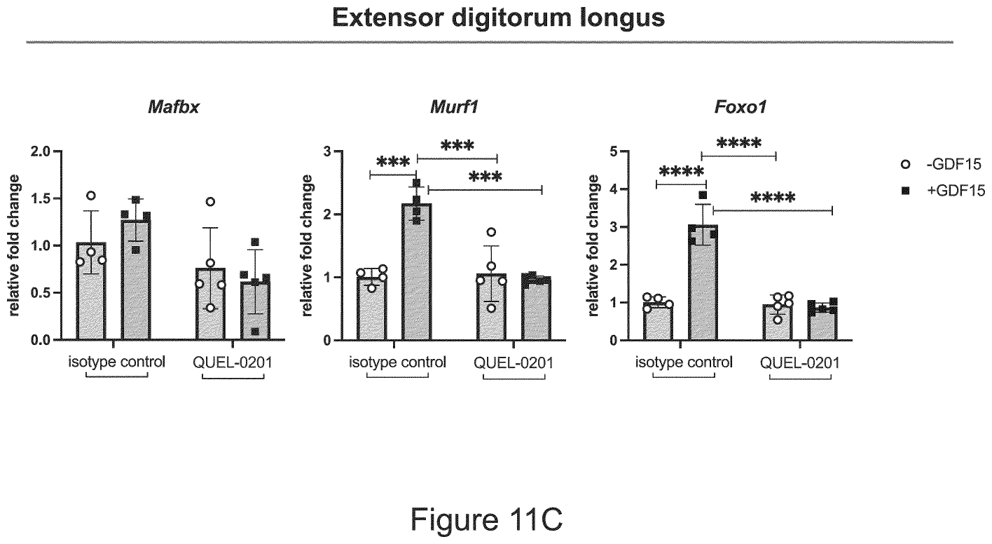

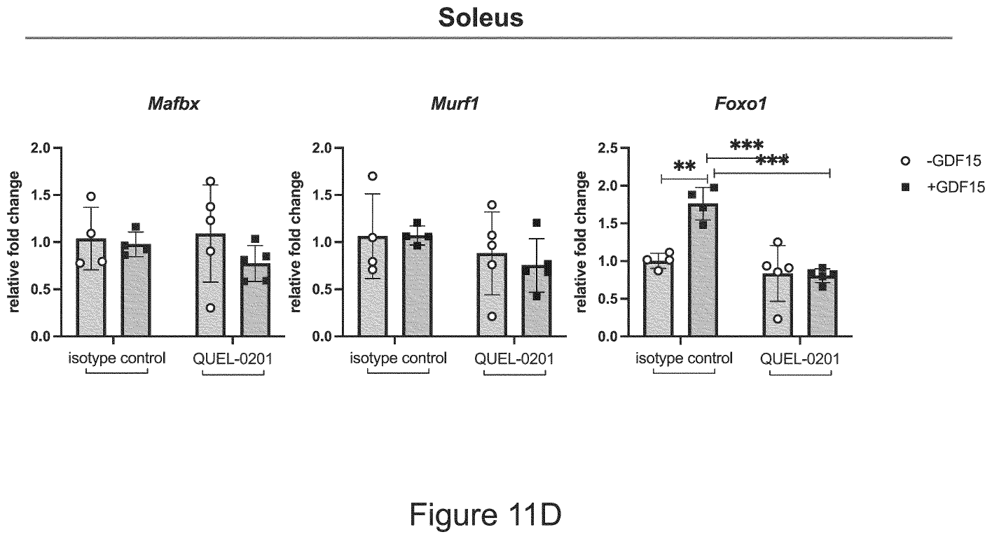

At the time point analysed, tissue from mice treated with human recombinant GDF15 and isotype control antibody showed a significant increase in expression of marker of muscle atrophy. No such effect was seen in any of the muscle tissue analysed when GDF15 was given after pretreatment with the anti-GFRAL antibody. FIG. 11.

### REFERENCES

¹ Li, Zhihua, Bingwei Wang, Xuefei Wu, Shi-Yuan Cheng, Luminita Paraoan, and Jiawei Zhou. 2005. ‘Identification, Expression and Functional Characterization of the GRAL Gene’. Journal of Neurochemistry 95 (2): 361-76. doi 10.1111/j.1471-4159.2005.03372.x.
² Emmerson, Paul J, Feng Wang, Yong Du, Qian Liu, Richard T Pickard, Malgorzata D Gonciarz, Tamer Coskun, et al. 2017. ‘The Metabolic Effects of GDF15 Are Mediated by the Orphan Receptor GFRAL’. Nature Medicine 23 (10): 1215-19. doi 10.1038/nm.4393
³ Hsu, Jer-Yuan, Suzanne Crawley, Michael Chen, Dina A. Ayupova, Darrin A. Lindhout, Jared Higbee, Alan Kutach, et al. 2017. ‘Non-Homeostatic Body Weight Regulation through a Brainstem-Restricted Receptor for GDF15’. Nature 550 (7675): 255-59. doi 10.1038/nature24042 and see also Hsu, et al. (2017). Erratum. Nature 551, 398
⁴ Mullican, Shannon E, Xiefan Lin-Schmidt, Chen-Ni Chin, Jose A Chavez, Jennifer L Furman, Anthony A Armstrong, Stephen C Beck, et al. 2017. ‘GFRAL Is the Receptor for GDF15 and the Ligand Promotes Weight Loss in Mice and Nonhuman Primates’. Nature Medicine 23 (10): 1150-57. doi 10.1038/nm.4392
⁵ Yang, Linda, Chih-Chuan Chang, Zhe Sun, Dennis Madsen, Haisun Zhu, Soren B Padkjor, Xiaoai Wu, et al. 2017. ‘GFRAL Is the Receptor for GDF15 and Is Required for the Anti-Obesity Effects of the Ligand’. Nature Medicine 23 (10): 1158-66. doi 10.1038/nm.4394
⁶ Xiong, Yumei, Kenneth Walker, Xiaoshan Min, Clarence Hale, Thanhvien Tran, Renee Komorowski, Jerry Yang, et al. 2017. ‘Long-Acting MIC-1/GDF15 Molecules to Treat Obesity: Evidence from Mice to Monkeys’. Science Translational Medicine 9 (412) doi 10.1126/scitranslmed.aan8732
⁷ Hsiao, Edward C., Leonidas G. Koniaris, Teresa Zimmers-Koniaris, Suzanne M. Sebald, Thanh V. Huynh, and Se-Jin Lee. 2000. ‘Characterization of Growth-Differentiation Factor 15, a Transforming Growth Factor β Superfamily Member Induced Following Liver Injury’. Molecular and Cellular Biology 20 (10): 3742-3751
⁸ Park, H., Kim, C. H., Jeong, J. H., Park, M., and Kim, K. S. (2016). GDF15 contributes to radiation-induced senescence through the ROS-mediated p16 pathway in human endothelial cells. Oncotarget 7, 9634-9644
⁹ Zheng, H., Wu, Y., Guo, T., Liu, F., Xu, Y., and Cai, S. (2020). Hypoxia Induces Growth Differentiation Factor 15 to Promote the Metastasis of Colorectal Cancer via PERK-elF2alpha Signaling. Biomed Res Int 2020, 5958272
¹⁰ Abulizi, Palida, Neruja Loganathan, Duo Zhao, Tina Mele, Yixin Zhang, Terry Zwiep, Kexiang Liu, and Xiufen Zheng. 2017. ‘Growth Differentiation Factor-15 Deficiency Augments Inflammatory Response and Exacerbates Septic Heart and Renal Injury Induced by Lipopolysaccharide’. Scientific Reports 7 (1): 1037. doi 10.1038/s41598-017-00902-5
¹¹ Johnen, Heiko, Shu Lin, Tamara Kuffner, David A Brown, Vicky Wang-Wei Tsai, Asne R Bauskin, Liyun Wu, et al. 2007. ‘Tumor-Induced Anorexia and Weight Loss Are Mediated by the TGF-β Superfamily Cytokine MIC-1’. Nature Medicine 13 (11): 1333-40. doi 10.1038/nm1677
¹² Brown, David A., John Moore, Heiko Johnen, Tom J. Smeets, Asne R. Bauskin, Tamara Kuffner, Helen Weedon, et al. 2007. ‘Serum Macrophage Inhibitory Cytokine 1 in Rheumatoid Arthritis: A Potential Marker of Erosive Joint Destruction’. Arthritis & Rheumatism 56 (3): 753-64. doi 10.1 002/art.2241 0
¹³ Breit, Samuel N., Juan J. Carrero, Vicky Wang-Wei Tsai, Nasreen Yagoutifam, Wei Luo, Tamara Kuffner, Asne R. Bauskin, et al. 2012. ‘Macrophage Inhibitory Cytokine-1 (MIC-1/GDF15) and Mortality in End-Stage Renal Disease’. Nephrology Dialysis Transplantation 27 (1): 70-75. doi 10.1 093/ndt/gfr575
¹⁴ Ruparelia, Neil, Joshua T. Chai, Edward A. Fisher, and Robin P. Choudhury. 2017. ‘Inflammatory Processes in Cardiovascular Disease: A Route to Targeted Therapies’. Nature Reviews Cardiology 14 (3): 133-44. doi 10.1038/nrcardio.2016.185
¹⁵ Hesselink, Matthijs K. C., Vera Schrauwen-Hinderling, and Patrick Schrauwen. 2016. ‘Skeletal Muscle Mitochondria as a Target to Prevent or Treat Type 2 Diabetes Mellitus’. Nature Reviews Endocrinology 12 (11): 633-45. doi 10.1038/nrendo.2016.104
¹⁶ Okazaki, Ryuji, Yuseok Moon, Toshiyuki Norimura, and Thomas Eling. 2006. ‘Ionizing Radiation Enhances the Expression of the Nonsteroidal Anti-Inflammatory Drug-Activated Gene (NAG1) by Increasing the Expression of TP53 in Human Colon Cancer Cells’. Radiation Research 165 (2): 125-30. doi 10.1667/RR3492.1
¹⁷ Moritake, Takashi, Hidetoshi Fujita, Mitsuru Yanagisawa, Miyako Nakawatari, Kaori Imadome, Etsuko Nakamura, Mayumi Iwakawa, and Takashi Imai. 2012. ‘Strain-Dependent Damage in Mouse Lung After Carbon Ion Irradiation’. International Journal of Radiation Oncology*Biology*Physics 84 (1): e95-102. doi 10.1016/j.ijrobp.2012.02.013
¹⁸ Tsai, Vicky Wang-Wei, Laurence Macia, Heiko Johnen, Tamara Kuffner, Rakesh Manadhar, Sebastian Beck Jorgensen, Ka Ki Michelle Lee-Ng, et al. 2013. ‘TGF-b Superfamily Cytokine MIC-1/GDF15 Is a Physiological Appetite and Body Weight Regulator’. Edited by Christopher Morrison. PLoS ONE8 (2): e55174. doi 10.1371/journal.pone.0055174
¹⁹ Fearon, Kenneth, Florian Strasser, Stefan D Anker, Ingvar Bosaeus, Eduardo Bruera, Robin L Fainsinger, Aminah Jatoi, et al. 2011. ‘Definition and Classification of Cancer Cachexia: An International Consensus’. The Lancet Oncology 12 (5): 489-95. doi 10.1016/S1470-2045(10)70218-7
²⁰ Anker, Markus S., Richard Holcomb, Maurizio Muscaritoli, Stephan von Haehling, Wilhelm Haverkamp, Aminah Jatoi, John E. Morley, et al. 2019. ‘Orphan Disease Status of Cancer Cachexia in the USA and in the European Union: A Systematic Review’. Journal of Cachexia, Sarcopenia and Muscle 10 (1): 22-34. doi 10.1002/jcsm.12402
²¹ Zhou, Ting, Bangyan Wang, Huiquan Liu, Kaixiang Yang, Sudip Thapa, Haowen Zhang, Lu Li, and Shiying Yu. 2018. ‘Development and Validation of a Clinically Applicable Score to Classify Cachexia Stages in Advanced Cancer Patients’. Journal of Cachexia, Sarcopenia and Muscle 9 (2): 306-14 doi 10.1002/jcsm.12275
²² Bourgeois, Brianna, Bo Fan, Neil Johannsen, Maria Cristina Gonzalez, Bennett K. Ng, Markus J. Sommer, John A. Shepherd, and Steven B. Heymsfield. 2019. ‘Improved Strength Prediction Combining Clinically Available Measures of Skeletal Muscle Mass and Quality’. Journal of Cachexia, Sarcopenia and Muscle 10 (1): 84-94. doi 10.1 002/jcsm.12353
²³ Senesse, Pierre, Eric Assenat, Stéphane Schneider, Cyrus Chargari, Nicolas Magné, David Azria, and Xavier Hébuterne. 2008. ‘Nutritional Support during Oncologic Treatment of Patients with Gastrointestinal Cancer: Who Could Benefit?’ Cancer Treatment Reviews 34 (6): 568-75. doi 10.1016/j.ctrv.2008.03.003
²⁴ Lerner, Lorena, Teresa G Hayes, Nianjun Tao, Brian Krieger, Bin Feng, Zhenhua Wu, Richard Nicoletti, M Isabel Chiu, Jeno Gyuris, and Jose M Garcia. 2015. ‘Plasma Growth Differentiation Factor 15 Is Associated with Weight Loss and Mortality in Cancer Patients’. Journal of Cachexia, Sarcopenia and Muscle 6 (4): 317-24. doi 10.1002/jcsm.12033
²⁵ Rocha, Ilanna Marques Gomes da, Aline Marcadenti, Galtieri Otdvio Cunha de Medeiros, Ricardo Andrade Bezerra, Juliana Florinda de Mendonga Rego, Maria Cristina Gonzalez, and Ana Paula Trussardi Fayh. 2019. ‘Is Cachexia Associated with Chemotherapy Toxicities in Gastrointestinal Cancer Patients? A Prospective Study’. Journal of Cachexia, Sarcopenia and Muscle 10 (2): 445-54. doi 10.1002/jcsm.12391
²⁶ Nicolini, Andrea, Paola Ferrari, Maria Chiara Masoni, Milena Fini, Stefania Pagani, Ottavio Giampietro, and Angelo Carpi. 2013. ‘Malnutrition, Anorexia and Cachexia in Cancer Patients: A Mini-Review on Pathogenesis and Treatment’. Biomedicine & Pharmacotherapy 67 (8): 807-17. doi 10.1016/j.biopha.2013.08.005
²⁷ Baracos, Vickie E., Lisa Martin, Murray Korc, Denis C. Guttridge, and Kenneth C. H. Fearon. 2018. ‘Cancer-Associated Cachexia’. Nature Reviews Disease Primers 4 (1): 1-18 doi 10.1038/nrdp.2017.105
²⁸ Pausch, Thomas, Werner Hartwig, Ulf Hinz, Thomas Swolana, Bogota D. Bundy, Thilo Hackert, Lars Grenacher, Markus W. Buchler, and Jens Werner. 2012. ‘Cachexia but Not Obesity Worsens the Postoperative Outcome after Pancreatoduodenectomy in Pancreatic Cancer’. Surgery, Topics in Pancreatic Surgery: A Festschrift Honoring Andrew L. Warshaw, 152 (3, Supplement): S81-88. doi 10.1016/j.surg.2012.05.028
²⁹ Bo, Yacong, Mingjie Yao, Ling Zhang, Wolde Bekalo, Weiquan Lu, and Quanjun Lu. 2015. ‘Preoperative Nutritional Risk Index to Predict Postoperative Survival Time in Primary Liver Cancer Patients’. Asia Pacific Journal of Clinical Nutrition 24 (4): 591-97. doi 10.6133/apjcn.2015.24.4.26
³⁰ Lerner, Lorena, Julie Tao, Qing Liu, Richard Nicoletti, Bin Feng, Brian Krieger, Elizabeth Mazsa, et al. 2016. ‘MAP3K11/GDF15 Axis Is a Critical Driver of Cancer Cachexia’. Journal of Cachexia, Sarcopenia and Muscle 7 (4): 467-82 2016 doi 10.1002/jcsm.12077
³¹ Tsai, Vicky W. W., Yasmin Husaini, Rakesh Manandhar, K. K. Michelle Lee-Ng, Hong Ping Zhang, Kate Harriott, Lele Jiang, et al. 2012. ‘Anorexia/Cachexia of Chronic Diseases: A Role for the TGF-β Family Cytokine MIC-1/GDF15’. Journal of Cachexia, Sarcopenia and Muscle 3 (4): 239-43. doi 10.1007/s13539-012-0082-6
³² Suriben, Rowena, Michael Chen, Jared Higbee, Julie Oeffinger, Richard Ventura, Betty Li, Kalyani Mondal, et al. 2020. ‘Antibody-Mediated Inhibition of GDF15-GFRAL Activity Reverses Cancer Cachexia in Mice’. Nature Medicine 26 (8): 1264-70. doi 10.1038/s41591-020-0945-x
³³ Macia, Laurence, Vicky Wang-Wei Tsai, Amy D. Nguyen, Heiko Johnen, Tamara Kuffner, Yan-Chuan Shi, Shu Lin, et al. 2012. ‘Macrophage Inhibitory Cytokine 1 (MIC-1/GDF15) Decreases Food Intake, Body Weight and Improves Glucose Tolerance in Mice on Normal & Obesogenic Diets’. Edited by Marcia B. Aguila. PLoS ONE 7 (4): e34868 2012 doi 10.1371/journal.pone.0034868
³⁴ Borner, Tito, Myrtha Arnold, Johan Ruud, Samuel N. Breit, Wolfgang Langhans, Thomas A. Lutz, Anders Blomqvist, and Thomas Riediger. 2017. ‘Anorexia-cachexia Syndrome in Hepatoma Tumour-bearing Rats Requires the Area Postrema but Not Vagal Afferents and Is Paralleled by Increased MIC-1/GDF15’. Journal of Cachexia, Sarcopenia and Muscle 8 (3): 417-27. doi 10.1002/jcsm.12169
³⁵ Tito Borner, Evan D. Shaulson, Misgana Y. Ghidewon, and Bart C. De Jonghe. 2020. ‘GDF15 Induces Anorexia through Nausea and Emesis’. Cell Metab, February. doi 10.1016/j.cmet.2019.12.004
³⁶ Fejzo, Marlena S., Olga V. Sazonova, J. Fah Sathirapongsasuti, Ingileif B. Hallgrímsdóttir, Vladimir Vacic, Kimber W. MacGibbon, Frederic P. Schoenberg, et al. 2018. ‘Placenta and Appetite Genes GDF15 and IGFBP7 Are Associated with Hyperemesis Gravidarum’. Nature Communications 9 (1). doi 10.1038/s41467-018-03258-0
³⁷ Rasmuson T, et al Increased serum cortisol levels are associated with high tumour grade in patients with renal cell carcinoma. ActaOncol 2001; 40: 83-87
³⁸ Drott C et al. Increased urinary excretion of cortisol and catecholamines in malnourished cancer patients. Ann Surg 1988; 208:645/50.27
³⁹ Schaur R J, et al. Tumor host relations. II. Influence of tumor extent and tumor site on plasma cortisol of patients with malignant diseases. J Cancer Res Clin Oncol 1979; 93:287/92
⁴⁰ Herman, J. P., McKlveen, J. M., Ghosal, S., Kopp, B., Wulsin, A., Makinson, R., Scheimann, J., and Myers, B. (2016). Regulation of the Hypothalamic-Pituitary-Adrenocortical Stress Response. Compr Physiol 6, 603-621
⁴¹ Turnbull, A. V., and Rivier, C. L. (1999). Regulation of the hypothalamic-pituitary-adrenal axis by cytokines: actions and mechanisms of action. Physiol Rev 79, 1-71
⁴² Lockhart, S. M., Saudek, V., and O'Rahilly, S. (2020). GDF15: A Hormone Conveying Somatic Distress to the Brain. Endocr Rev 41
⁴³ Shimada, K., and Mitchison, T. J. (2019). Unsupervised identification of disease states from high-dimensional physiological and histopathological profiles. Mol Syst Biol 15, e8636
⁴⁴ Breit, S. N., Brown, D. A., and Tsai, V. W. (2020). The GDF15-GFRAL Pathway in Health and Metabolic Disease: Friend or Foe? Annu Rev Physiol
⁴⁵ Akiyama, Y., Yoshimura, M., Ueno, H., Sanada, K., Tanaka, K., Sonoda, S., Nishimura, H., Nishimura, K., Motojima, Y., Saito, R., et al. (2020). Peripherally administered cisplatin activates a parvocellular neuronal subtype expressing arginine vasopressin and enhanced green fluorescent protein in the paraventricular nucleus of a transgenic rat. J Physiol Sci 70, 35
⁴⁶ Kenward, H., Pelligand, L., and Elliott, J. (2014). Assessment of low-dose cisplatin as a model of nausea and emesis in beagle dogs, potential for repeated administration. Exp Brain Res 232, 2685-2697
⁴⁷ Breen, D. M., Kim, H., Bennett, D., Calle, R. A., Collins, S., Esquejo, R. M., He, T., Joaquim, S., Joyce, A., Lambert, M., et al. (2020). GDF-15 Neutralization Alleviates Platinum-Based Chemotherapy-Induced Emesis, Anorexia, and Weight Loss in Mice and Nonhuman Primates. Cell Metab 32, 938-950 e936
⁴⁸ Kim, K. H., Kim, S. H., Han, D. H., Jo, Y. S., Lee, Y. H., and Lee, M. S. (2018). Growth differentiation factor 15 ameliorates nonalcoholic steatohepatitis and related metabolic disorders in mice. Sci Rep 8, 6789
⁴⁹ Patel, S., Alvarez-Guaita, A., Melvin, A., Rimmington, D., Dattilo, A., Miedzybrodzka, E. L., Cimino, I., Maurin, A. C., Roberts, G. P., Meek, C. L., et al. (2019). GDF15 Provides an Endocrine Signal of Nutritional Stress in Mice and Humans. Cell Metab 29, 707-718 e708
⁵⁰ Lin C L, Wu T J, Machacek D A, et al. 1997 Urine free cortisol and cortisone determined by high performance liquid chromatography in the diagnosis of Cushing syndrome. J Clin Endocrinol Metab. 82:151-155
⁵¹ Tsai et al PLOS ONE 10, e0133362. doi:10.1371/journal.pone.0133362
⁵² Bodine S C, Baehr L M. Am J Physiol Endocrinol Metab. 2014 Sep. 15; 307(6):E469-84
⁵³ Obayu et al., FASEB J. 2022 February; 36(2):e22152. doi: 10.1096/fj.202101385RR.

### Sequence Information

TABLE A
Table A below shows variable domain sequences of antibodies described in this
specification. All QUEL VH domains, QUEL VL domains, QUEL CDRs, QUEL heavy chains and
QUEL light chains, antibodies comprising them, as well as their encoding nucleic acids,
represent embodiments of the present invention.

| ID | Name | Sequence |
|---|---|---|
| 1 | QUEL-0101 | CAGGTTCAACTGGTGGAGTCTGGGGGAGGCGTGGTCCAGCCTGG |
| | VH domain nucleotide | GAGGTCCCTGAGACTCTCCTGTGCAGCCTCTGGATTCGCCTTCA |
| | | ATAACTATGGCATGCACTGGGTCCGCCAGGGTCCAGGCAGGGGG |
| | | CTGGAATGGGTGGCTATTATATCATATGATGGAACAACTCAATA |
| | | CTCTGTAGACTCCGTGAAGGGCCGATTCACCATCTCCAGAGACA |
| | | ATTCCAAGAACACGTTGTATCTGCAAATGAACAGCCTGAGAACT |
| | | GAGGACACGGCTCTATATTACTGTGCGAAAGAGGCGGGGGATTT |
| | | CTATGGTTCGGGGAATTATGGGTACCACAAGTATGGTTTGGACG |
| | | TCTGGGGCCAAGGGACCACGGTCACCGTCTCCTCAG |
| 2 | QUEL-0101 | QVQLVESGGGVVQPGRSLRLSCAASGFAFNNYGMHWVRQGPGRG |
| | VH domain amino acid | LEWVAIISYDGTTQYSVDSVKGRFTISRDNSKNTLYLQMNSLRT |
| | | EDTALYYCAKEAGDFYGSGNYGYHKYGLDVWGQGTTVTVSS |
| 3 | QUEL-0101 HCDR1 | GFAFNNYG |
| 4 | QUEL-0101 HCDR2 | ISYDGTTQ |
| 5 | QUEL-0101 HCDR3 | AKEAGDFYGSGNYGYHKYGLDV |
| 6 | QUEL-0101 | GACATCCAGATGACCCAGTCTCCATCCTCCCTGTCTGCATCTGT |
| | VL domain nucleotide | AGGAGACAGAGTCACCATCACTTGCCGGGCGAGTCAGGACATTA |
| | | GCAATTATTTAGCCTGGTATCAGCAGAAGCCAGGGAAAATTCCT |
| | | AAGCTCCTGATCTATGCTGCATCCACTTTGCAATCAGGGGTCCC |
| | | ATCTCGGTTCAGTGGCAGTGGATCTGGGACAGATTTCACTCTCA |
| | | CCATCAGCAGCCTGCAGCCTGAAGATGTTGCAAGTTATTACTGT |
| | | CAAAAATATAGCAGTGCCCCGCTCACTTTCGGCGGAGGGACCAA |
| | | GGTGGAGATCAAAC |
| 7 | QUEL-0101 | DIQMTQSPSSLSASVGDRVTITCRASQDISNYLAWYQQKPGKIP |
| | VL domain amino acid | KLLIYAASTLQSGVPSRFSGSGSGTDFTLTISSLQPEDVASYYC |
| | | QKYSSAPLTFGGGTKVEIK |
| 8 | QUEL-0101 LCDR1 | QDISNY |
| 9 | QUEL-0101 LCDR2 | AAS |
| 10 | QUEL-0101 LCDR3 | QKYSSAPLT |
| 97 | QUEL-0102 | CAGGTTCAACTGGTGGAGTCTGGGGGAGGCGTGGTCCAGCCTGG |
| | VH domain nucleotide | GAGGTCCCTGAGACTCTCCTGTGCAGCCTCTGGATTCACCCTCA |
| | | AAAACTATGGCATGCACTGGGTCCGCCAGGGTCCAGGCAAGGGG |
| | | CTGGAGTGGGTGGCAATTATATCATATGATGGAACATTTAAATA |
| | | TTCTGTAGACTCCGTGAAGGGCCGATTCACCATCTCCAGAGACA |
| | | ATTCCAAGAACACGTTGTATCTGCAAATGAACAGCCTGAGAACT |
| | | GAGGACACGGCTCTGTATTACTGTGCGAAAGAGGCGGGGGATTT |
| | | CTATGGTTCGGGGAGTTATGGGTACCACAAGTATGGTTTGGACG |
| | | TCTGGGGCCAAGGGACCACGGTCACCGTCTCCTCAG |
| 98 | QUEL-0102 | QVQLVESGGGVVQPGRSLRLSCAASGFTLKNYGMHWVRQGPGKG |
| | VH domain amino acid | LEWVAIISYDGTFKYSVDSVKGRFTISRDNSKNTLYLQMNSLRT |
| | | EDTALYYCAKEAGDFYGSGSYGYHKYGLDVWGQGTTVTVSS |
| 99 | QUEL-0102 HCDR1 | GFTLKNYG |
| 100 | QUEL-0102 HCDR2 | ISYDGTFK |
| 101 | QUEL-0102 HCDR3 | AKEAGDFYGSGSYGYHKYGLDV |
| 102 | QUEL-0102 | GACGTCCAGATGACCCAGTCTCCATCCTCCCTGTCTGCATCTGT |
| | VL domain nucleotide | AGGAGACAGAGTCACCATCACTTGCCGGGCGAGTCAGGACATTA |
| | | GCAATTATTTAGCCTGGTATCAGCAGCAACCAGGGAAAGTTCCT |
| | | AAACTCCTGATCTATGCTGCATCCACTTTGCAATCAGGGGTCCC |
| | | ATCTCGGTTCAGTGGCAGTGGATCTGGGACAGATTTCACTCTCA |
| | | CCATCAGCAGCCTGCAGCCTGAAGATGTTGCAAGTTATTACTGT |
| | | CAAAAATATAACAGTGCCCCGCTCACTTTCGGCGGAGGGACCGA |
| | | GGTGGAGATCAAGC |
| 103 | QUEL-0102 | DVQMTQSPSSLSASVGDRVTITCRASQDISNYLAWYQQQPGKVP |
| | VL domain amino acid | KLLIYAASTLQSGVPSRFSGSGSGTDFTLTISSLQPEDVASYYC |
| | | QKYNSAPLTFGGGTEVEIK |
| 8 | QUEL-0102 LCDR1 | QDISNY |
| 9 | QUEL-0102 LCDR2 | AAS |
| 104 | QUEL-0102 LCDR3 | QKYNSAPLT |
| 105 | QUEL-0103 | CAGGTTCAACTGGTGGAGTCTGGGGGAGGCGTGGTCCAGCCTGG |
| | VH domain nucleotide | GAGGTCCCTGAGACTCTCCTGTGCAGCCTCTGGATTCACCTTCA |
| | | ATAACTATGGCATGCACTGGGTCCGCCAGGGTCCAGGCAAGGGG |
| | | CTGGAGTGGGTGGCTATTATATCATATGATGGAACAACTAAATA |
| | | CTCTGTAGACTCCGTGAAGGGCCGATTCACCATCTCCAGAGACA |
| | | ATTCCAAGAACACGTTGTATCTACAAATGAACAGCCTGAGAACT |
| | | GAGGACACGGCTCTGTATTTCTGTGCGAAAGAGGCGGGGGATTT |
| | | CTATGGTTCGGGGAATTATGGGTACCACAAGTATGGTTTGGACG |
| | | TCTGGGGCCAAGGGACCACGGTCACCGTCTCCTCAG |
| 106 | QUEL-0103 | QVQLVESGGGVVQPGRSLRLSCAASGFTFNNYGMHWVRQGPGKG |
| | VH domain amino acid | LEWVAIISYDGTTKYSVDSVKGRFTISRDNSKNTLYLQMNSLRT |
| | | EDTALYFCAKEAGDFYGSGNYGYHKYGLDVWGQGTTVTVSS |
| 107 | QUEL-0103 HCDR1 | GFTFNNYG |
| 108 | QUEL-0103 HCDR2 | ISYDGTTK |
| 5 | QUEL-0103 HCDR3 | AKEAGDFYGSGNYGYHKYGLDV |
| 109 | QUEL-0103 | GACATCCAGATGACCCAGTCTCCATCCTCCCTGTCTGCATCTGT |
| | VL domain nucleotide | AGGAGACAGAGTCACCATCACTTGCCGGGCGAGTCAGGACATTA |
| | | GCAATTATTTAGCCTGGTATCAGCAGAAACCAGGGAAAGTTCCT |
| | | AAGCTCCTGATCTATGCTGCATCCACTTTGCAATCAGGGGTCCC |
| | | ATCTCGGTTCAGTGGCAGTGGATCTGGGACAGATTTCACTCTCA |
| | | CCATCAGCAGCCTGCAGCCTGAAGATGTTGCAAGTTATTACTGT |
| | | CAAAAATATAACAGTGCCCCGCTCACTTTCGGCGGAGGGACCGA |
| | | GGTGGAGATCAAGC |
| 110 | QUEL-0103 | DIQMTQSPSSLSASVGDRVTITCRASQDISNYLAWYQQKPGKVP |
| | VL domain amino acid | KLLIYAASTLQSGVPSRFSGSGSGTDFTLTISSLQPEDVASYYC |
| | | QKYNSAPLTFGGGTEVEIK |
| 8 | QUEL-0103 LCDR1 | QDISNY |
| 9 | QUEL-0103 LCDR2 | AAS |
| 104 | QUEL-0103 LCDR3 | QKYNSAPLT |
| 111 | QUEL-0104 | CAGGTTCAACTGGTGGAGTCTGGGGGAGGCGTGGTCCAGCCTGG |
| | VH domain nucleotide | GAGGTCCCTGAGACTCTCCTGTGCAGCCTCTGGATTCACCTTCA |
| | | ATAACTATGGCATGCACTGGGTCCGCCAGGGTCCAGGCAAGGGG |
| | | CTGGAGTGGGTGGCTATTATATCATATGATGGAACAACTAAATA |
| | | CTCTGTAGACTCCGTGAAGGGCCGATTCACTATCTCCAGAGACA |
| | | ATTCCAAGAACACGTTGTATATGCAAATGGACAGCCTGAGAACG |
| | | GAGGACACGGCTCTGTATTACTGTGCGAAGGAGGCGGGGGATTT |
| | | CTATGGTTCGGGGAATTATGGGTACCTCTACTATGGTTTGGACG |
| | | TCTGGGGCCAAGGGACCACGGTCACCGTCTCCTCAG |
| 112 | QUEL-0104 | QVQLVESGGGVVQPGRSLRLSCAASGFTENNYGMHWVRQGPGKG |
| | VH domain amino acid | LEWVAIISYDGTTKYSVDSVKGRFTISRDNSKNTLYMQMDSLRT |
| | | EDTALYYCAKEAGDFYGSGNYGYLYYGLDVWGQGTTVTVSS |
| 107 | QUEL-0104 HCDR1 | GFTENNYG |
| 108 | QUEL-0104 HCDR2 | ISYDGTTK |
| 113 | QUEL-0104 HCDR3 | AKEAGDFYGSGNYGYLYYGLDV |
| 114 | QUEL-0104 | GACATCCAGATGACCCAGTCTCCATCCTCCCTGTCTGCATCTGT |
| | VL domain nucleotide | AGGAGACAGAGTCACCATCACTTGCCGGGCGAGTCAGGACATTA |
| | | GCAATTATTTAGCCTGGTATCAGCAGAAACCAGGGAAAGTTCCT |
| | | AACCTCCTGATCTTTGCTGCATCCACTTTGCAATCAGGGGTCCC |
| | | ATCTCGGTTCAGTGGCAGTGGATCTGGGACAGATTTCACTCTCA |
| | | CCATCAGCAGCCTGCAGCCTGAAGATGTTGCAACTTATTACTGT |
| | | CAAAAGTATAATAGTGCCCCGCTCACTTTCGGCGGAGGGACCAA |
| | | GGTGGAGATCAACC |
| 115 | QUEL-0104 | DIQMTQSPSSLSASVGDRVTITCRASQDISNYLAWYQQKPGKVP |
| | VL domain amino acid | NLLIFAASTLQSGVPSRFSGSGSGTDFTLTISSLQPEDVATYYC |
| | | QKYNSAPLTFGGGTKVEIN |
| 8 | QUEL-0104 LCDR1 | QDISNY |
| 9 | QUEL-0104 LCDR2 | AAS |
| 104 | QUEL-0104 LCDR3 | QKYNSAPLT |
| 116 | QUEL-0105 | CAGGTGCAACTGGTGGAGTCTGGGGGAGACGTGGTCCAGCCTGG |
| | VH domain nucleotide | GAGGTCCCTGAGACTCTCCTGTGCAGCCTCTGGTTTCACCTTCA |
| | | AAAACTATGGCATGCACTGGGTCCGCCAGGGTCTAGGCAAGGGG |
| | | CTGGAGTGGGTGGCTATTATATCATATGATGGAACAACTAAATA |
| | | CTCTGTAGACTCCGTGAAGGGCCGATTCACCATCTCCAGAGACA |
| | | ATTCCAAGAACACGTTGTATCTGCAAATGAACAGCCTGAGAACT |
| | | GAGGACACGGCTCTGTATTACTGTGCGAAAGAGGCGGGGGATTT |
| | | CTATGGTTCGGGGAATTATGGGTACTACTACTATGGTTTGGACG |
| | | TCTGGGGCCAAGGGACCACGGTCACCGTCTCCTCAG |
| 117 | QUEL-0105 | QVQLVESGGDVVQPGRSLRLSCAASGFTFKNYGMHWVRQGLGKG |
| | VH domain amino acid | LEWVAIISYDGTTKYSVDSVKGRFTISRDNSKNTLYLQMNSLRT |
| | | EDTALYYCAKEAGDFYGSGNYGYYYYGLDVWGQGTTVTVSS |
| 118 | QUEL-0105 HCDR1 | GFTFKNYG |
| 108 | QUEL-0105 HCDR2 | ISYDGTTK |
| 119 | QUEL-0105 HCDR3 | AKEAGDFYGSGNYGYYYYGLDV |
| 120 | QUEL-0105 | GACATCCAGATGACCCAGTCTCCATCCTCCCTGTCTGCATCTGT |
| | VL domain nucleotide | AGGAGACAGAGTCACCATCACTTGCCGGGCGAATCAGGACATTA |
| | | GCAATTATTTAGCCTGGTATCAGCAGAAACCAGGGAAATTTCCT |
| | | AAGCTCCTGATCTATGCTGCATCCACTTTGCAATCAGGGGTCCC |
| | | ATCTCGGTTCAGTGGCAGTGGATCTGGGACAGATTTCACTCTCA |
| | | CCATCAGCAGCCTGCAGCCTGAAGATGTTGCAACTTATTACTGT |
| | | CAAAAGTATAACAGTGCCCCGCTCACTTTCGGCGGAGGGACCAA |
| | | GGTGGAGATCAAAC |
| 121 | QUEL-0105 | DIQMTQSPSSLSASVGDRVTITCRANQDISNYLAWYQQKPGKFP |
| | VL domain amino acid | KLLIYAASTLQSGVPSRFSGSGSGTDFTLTISSLQPEDVATYYC |
| | | QKYNSAPLTFGGGTKVEIK |
| 8 | QUEL-0105 LCDR1 | QDISNY |
| 9 | QUEL-0105 LCDR2 | AAS |
| 104 | QUEL-0105 LCDR3 | QKYNSAPLT |
| 11 | QUEL-0201 | CAGGTCCAGCTTGTGCAGTCTGGGGCTGAGGTGAAGGAGCCTGG |
| | VH domain nucleotide | GGCCTCAGTGAGGGTTTCCTGTAAGGCTTCTGGATACACCTTCA |
| | | TTAGTCACGCTATACATTGGGTGCGCCAGGCCCCCGGACAAAGA |
| | | CTTGAGTGGATGGGATGGATCAACGCTGTCAATGGAAACACAAA |
| | | ATATTCACAGAAGTTCCAGGACAGAGTCACCTTTACCAGGGACA |
| | | CATCCGCGAGCACAGCCTACATGGACCTGAACAGCCTGAGAGCT |
| | | GAAGACACGGCTCTTTATTACTGTGCGAGAGAGGGATACACCTA |
| | | TGGTGTCCACTACTCGTACGGTATGGACGTCTGGGGCCAAGGGA |
| | | CCACGGTCACCGTCTCGTCAG |
| 12 | QUEL-0201 | QVQLVQSGAEVKEPGASVRVSCKASGYTFISHAIHWVRQAPGQR |
| | VH domain amino acid | LEWMGWINAVNGNTKYSQKFQDRVTFTRDTSASTAYMDLNSLRA |
| | | EDTALYYCAREGYTYGVHYSYGMDVWGQGTTVTVSS |
| 13 | QUEL-0201 HCDR1 | GYTFISHA |
| 14 | QUEL-0201 HCDR2 | INAVNGNT |
| 15 | QUEL-0201 HCDR3 | AREGYTYGVHYSYGMDV |
| 16 | QUEL-0201 | CAGTCTGTGCTGACGCAGCCGCCCTCAGTGTCTGGGGCCCCAGG |
| | VL domain nucleotide | GCAGAGGGTCACCATCTCCTGCACTGGGAGCAGTTCCAATATCG |
| | | GGGCAGGTTATGATGTACACTGGTACCAGCAGCTTCCAGGAACA |
| | | GTCCTCAAACTCCTCATTTATGGTAACAACAATCGGCCCTCAGG |
| | | GGTCCCTGACCGATTCTCTGGCTCCAACTCTGGCACCTCAGCCT |
| | | CCCTGGCCATCACTGGGCTCCAGCCTGAGGATGAGGCTGATTAT |
| | | TACTGCCAGTCCTATGACAGCAGCCTGAGTGGTTATTGGGTGTT |
| | | CGGCGGAGGGACCAAGCTGACCGTCCTAG |
| 17 | QUEL-0201 | QSVLTQPPSVSGAPGQRVTISCTGSSSNIGAGYDVHWYQQLPGT |
| | VL domain amino acid | VLKLLIYGNNNRPSGVPDRESGSNSGTSASLAITGLQPEDEADY |
| | | YCQSYDSSLSGYWVFGGGTKLTVL |
| 18 | QUEL-0201 LCDR1 | SSNIGAGYD |
| 19 | QUEL-0201 LCDR2 | GNN |
| 20 | QUEL-0201 LCDR3 | QSYDSSLSGYWV |
| 21 | QUEL-0301 | GAGGTGCAACTGGTGGAGTCTGGGGGAGGCTTGGTCCAGCCTGG |
| | VH domain nucleotide | GGGGTCCCTGAGACTCTCCTGCGCAGCTTCTGGATTCACCTTTA |
| | | GTAACTCTTGGATGAACTGGGTCCGCCAGACTCCAGGGAAGGGG |
| | | CTGGAGTGGGTGGCCAATATAGACCAAGATGGAGATCAGAAATA |
| | | CTATGTGGACTCTGTGAAGGGCCGATTCACCATCTCCAGAGACA |
| | | ACGCCAAGAACTCACTGTATCTGCAAATGAACAGCCTGAGAGCC |
| | | GAGGACACGGCTGTCTATTACTGTGCGAGAGAAATAACTGGAAC |
| | | TACATTTGACTACTGGGGCCAGGGAACCCTGGTCACCGTCTCCT |
| | | CAG |
| 22 | QUEL-0301 | EVQLVESGGGLVQPGGSLRLSCAASGFTFSNSWMNWVRQTPGKG |
| | VH domain amino acid | LEWVANIDQDGDQKYYVDSVKGRFTISRDNAKNSLYLQMNSLRA |
| | | EDTAVYYCAREITGTTEDYWGQGTLVTVSS |
| 23 | QUEL-0301 HCDR1 | GFTFSNSW |
| 24 | QUEL-0301 HCDR2 | IDQDGDQK |
| 25 | QUEL-0301 HCDR3 | AREITGTTEDY |
| 26 | QUEL-0301 | CAGTCTGTGCTGACTCAGCCACCCTCAGCGTCTGGGACCCCCGG |
| | VL domain nucleotide | GCAGAGGGTCACCATCTCTTGTTCTGGAAGCAGCTCCAACATCG |
| | | GAAGTAATATTGTAAACTGGTACCAGCAACTCCCAGGAACGGCC |
| | | CCCAAACTCCTCATCTCTAGTAATAATCAGCGGCCCTCAGGGGT |
| | | CCCTGACCGATTCTCTGGCTCCAAGTCTGGCACCTCAGCCTCCC |
| | | TGGCCATCAGTGGGCTCCAGTCTGAGGATGAGGCTGATTATTAC |
| | | TGTACAACATGGGATGACAGCCTGGATGGTCCGGTGTTCGGCGG |
| | | AGGGACCAAGCTGACCGTCCTGG |
| 27 | QUEL-0301 | QSVLTQPPSASGTPGQRVTISCSGSSSNIGSNIVNWYQQLPGTA |
| | VL domain amino acid | PKLLISSNNQRPSGVPDRFSGSKSGTSASLAISGLQSEDEADYY |
| | | CTTWDDSLDGPVFGGGTKLTVL |
| 28 | QUEL-0301 LCDR1 | SSNIGSNI |
| 29 | QUEL-0301 LCDR2 | SNN |
| 30 | QUEL-0301 LCDR3 | TTWDDSLDGPV |
| 122 | QUEL-0302 | GAGGTGCAGCTGGTGGAGTCTGGGGGAGGCTTGGTCCAGCCTGG |
| | VH domain nucleotide | GGGGTCCCTGAGACTCTCCTGTGCAGCCTCTGGATTCACCTTTA |
| | | GTAACTCTTGGATGAACTGGGTCCGCCAAGCTCCAGGGAAGGGG |
| | | CTGGAGTGGGTGGCCAACATAAACCAAGATGGCAGTGAGAGATA |
| | | CTATGTGGACTCTGTGAAGGGCCGATTCACCATCTCCAGAGACA |
| | | ACTCCAAGAACTCACTGTCTCTGCAAATGAACAGCCTGAGAGCC |
| | | GAGGACACGGCTATCTATTACTGTGCGAGAGAAATAACTGGAAC |
| | | CTACTTTGACTACTGGGGCCAGGGAACCCTGGTCACCGTCTCCT |
| | | CAG |
| 123 | QUEL-0302 | EVQLVESGGGLVQPGGSLRLSCAASGFTFSNSWMNWVRQAPGKG |
| | VH domain amino acid | LEWVANINQDGSERYYVDSVKGRFTISRDNSKNSLSLQMNSLRA |
| | | EDTAIYYCAREITGTYFDYWGQGTLVTVSS |
| 23 | QUEL-0302 HCDR1 | GFTFSNSW |
| 124 | QUEL-0302 HCDR2 | INQDGSER |
| 125 | QUEL-0302 HCDR3 | AREITGTYFDY |
| 126 | QUEL-0302 | CAGTCTGTGCTGACTCAGCCACCCTCAGCGTCTGGGACCCCCGG |
| | VL domain nucleotide | GCAGAGGGTCACCATCTCTTGTTCTGGAAGCAGCTCCAACATCG |
| | | GAAATAATTATGTATACTGGTACCAACAGCTCCCAGGAACGGCC |
| | | CCCAAACTCCTCATCTATAGGAATAATCAGCGGCCCTCAGGGGT |
| | | CCCTGACCGATTCTCTGGCTCCAAGTCTGGCACCTCAGCCTCCC |
| | | TGGCCATCAGTGGGCTCCGGTCCGAGGATGAGGCTGAATATTAC |
| | | TGTGCAGCATGGGATGACAGCCTGAGTGGTCCGGTGTTCGGCGG |
| | | AGGGACCAAGCTGACCGTCCTAG |
| 127 | QUEL-0302 | QSVLTQPPSASGTPGQRVTISCSGSSSNIGNNYVYWYQQLPGTA |
| | VL domain amino acid | PKLLIYRNNQRPSGVPDRESGSKSGTSASLAISGLRSEDEAEYY |
| | | CAAWDDSLSGPVFGGGTKLTVL |
| 128 | QUEL-0302 LCDR1 | SSNIGNNY |
| 129 | QUEL-0302 LCDR2 | RNN |
| 130 | QUEL-0302 LCDR3 | AAWDDSLSGPV |
| 131 | QUEL-0303 | GAGGTGCAGCTGGTGGAGTCTGGGGGAGGCTTGGTCCAGCCTGG |
| | VH domain nucleotide | GGGGTCCCTGAGACTCTCCTGTGCAGCCTCTGGATTCACCTTTA |
| | | GTAACTATTGGATGAACTGGGTCCGCCAGGCTCCAGGGAAGGGG |
| | | CTGGAGTGGGTGGCCAACATAGACCAAGATGGAAGTGAGAAATA |
| | | CTATGTGGACTCTTTGAAGGGCCGATTCACCATCTCCAGAGACA |
| | | ACGCCAAGAACTCACTGTATCTGCAAATGAACAGCCTGAGAGCC |
| | | GAGGACTCGGCTGTGTATTACTGTGCGAGAGAGCTAACTGGAAC |
| | | TACTTTTGACTACTGGGGCCAGGGAACCCTGGTCACCGTCTCCT |
| | | CAG |
| 132 | QUEL-0303 | EVQLVESGGGLVQPGGSLRLSCAASGFTFSNYWMNWVRQAPGKG |
| | VH domain amino acid | LEWVANIDQDGSEKYYVDSLKGRFTISRDNAKNSLYLQMNSLRA |
| | | EDSAVYYCARELTGTTFDYWGQGTLVTVSS |
| 133 | QUEL-0303 HCDR1 | GFTFSNYW |
| 134 | QUEL-0303 HCDR2 | IDQDGSEK |
| 135 | QUEL-0303 HCDR3 | ARELTGTTEDY |
| 136 | QUEL-0303 | CAGTCTGTGCTGACTCAGCCACCCTCAGCGTCTGGGACCCCCGG |
| | VL domain nucleotide | GCAGAGGGTCACCATCTCTTGTTCTGGAAGCAGCTCCAACATCG |
| | | GAAGTAATACTATAAACTGGTACCAACTGCTCCCAGGAACGGCC |
| | | CCCAAACTCCTCATCTATAGTAATAATCAGCGGCCCTCAGGGGT |
| | | CCCTGACCGATTCTCTGGCTCCAAGTCTGGCACCTCAGCCTCCC |
| | | TGGCCATCAGTGGGCTCCAGTCTGAGGATGAGGCTGATTATTAC |
| | | TGTGCAGCATGGGATGACAGCCTGAATGGTCCGGTGTTCGGCGG |
| | | AGGGACCAAGCTGACCGTCCTAG |
| 137 | QUEL-0303 | QSVLTQPPSASGTPGQRVTISCSGSSSNIGSNTINWYQLLPGTA |
| | VL domain amino acid | PKLLIYSNNQRPSGVPDRESGSKSGTSASLAISGLQSEDEADYY |
| | | CAAWDDSLNGPVFGGGTKLTVL |
| 138 | QUEL-0303 LCDR1 | SSNIGSNT |
| 29 | QUEL-0303 LCDR2 | SNN |
| 139 | QUEL-0303 LCDR3 | AAWDDSLNGPV |
| 140 | QUEL-0304 | GAGGTGCAGCTGGTGGAGTCTGGGGGAGGCTTGGTCCAGCCTGG |
| | VH domain nucleotide | GGGGTCCCTGAGACTCTCCTGTGCAGCCTCTGGATTCACATTTA |
| | | GTAACTATTGGATGAGCTGGGTCCGCCAGGCTCCAGGGAAGGGG |
| | | CTGGAGTGGGTGGCCAACATGAACCATGATGGAAGTGATAAATA |
| | | CTATGTGGACTCTGTGAAGGGCCGATTCACCATCTCCAGAGACA |
| | | ACGCCAAGAGTTTAATGTATCTGCAAATGAACAGCCTGAGAGCC |
| | | GAGGACACGGCTGTATATTACTGTGCGAGAGATCGGACTGGAAC |
| | | TATCTTTGACTACTGGGGCCAGGGAACCTTGGTCACCGTCTCCT |
| | | CAG |
| 141 | QUEL-0304 | EVQLVESGGGLVQPGGSLRLSCAASGFTFSNYWMSWVRQAPGKG |
| | VH domain amino acid | LEWVANMNHDGSDKYYVDSVKGRFTISRDNAKSLMYLQMNSLRA |
| | | EDTAVYYCARDRTGTIFDYWGQGTLVTVSS |
| 133 | QUEL-0304 HCDR1 | GFTFSNYW |
| 142 | QUEL-0304 HCDR2 | MNHDGSDK |
| 143 | QUEL-0304 HCDR3 | ARDRTGTIFDY |
| 144 | QUEL-0304 | CAGTCTGTGCTGACTCAGCCACCCTCAGCGTCTGGGACCCCCGG |
| | VL domain nucleotide | GCAGAGGGTCACCATCTCTTGTTCTGGAAGCAGCTCCAACATCG |
| | | GAAGTAATACTGTAAACTGGTACCAGCAACTCCCAGGAACGGCC |
| | | CCCAAAGTCCTCATCTATAGTAATAATCAGCGGCCCTCAGGGGT |
| | | CCCTGACCGATTCTCTGGCTCCAAGTCTGGCACCTCAGCCTCCC |
| | | TGGCCATCAGTGGGCTCCAGTCTGAGGATGAGGCTAATTATTAC |
| | | TGTGCAACATGGGATGACAGCCTGAATGGTCCGGTGTTCGGCGG |
| | | AGGGACCAAGCTGACCGTCCTAG |
| 145 | QUEL-0304 | QSVLTQPPSASGTPGQRVTISCSGSSSNIGSNTVNWYQQLPGTA |
| | VL domain amino acid | PKVLIYSNNQRPSGVPDRESGSKSGTSASLAISGLQSEDEANYY |
| | | CATWDDSLNGPVFGGGTKLTVL |
| 138 | QUEL-0304 LCDR1 | SSNIGSNT |
| 29 | QUEL-0304 LCDR2 | SNN |
| 146 | QUEL-0304 LCDR3 | ATWDDSLNGPV |

TABLE C
Table C below shows sequences of antibody heavy and light chain constant regions and nucleic acids encoding them. These are examples
of constant regions that may be used in antibodies of the present invention.

| ID | | | Description | Sequence |
|---|---|---|---|---|
| 33 | Human | IGHG1*01 | Human Heavy Chain Constant | gcctccaccaagggcccatcggtcttccccctggcaccctcctccaagagcacctctg |
| | IgG1 | | Region (IGHG1*01) | ggggcacagcggccctgggctgcctggtcaaggactacttccccgaaccggtgacggt |
| | constant | | Nucleotide Sequence | gtcgtggaactcaggcgccctgaccagcggcgtgcacaccttcccggctgtcctacag |
| | region | | | tcctcaggactctactccctcagcagcgtggtgaccgtgccctccagcagcttgggca |
| | | | | cccagacctacatctgcaacgtgaatcacaagcccagcaacaccaaggtggacaagaa |
| | | | | agttgagcccaaatcttgtgacaaaactcacacatgcccaccgtgcccagcacctgaa |
| | | | | ctcctggggggaccgtcagtcttcctcttccccccaaaacccaaggacaccctcatga |
| | | | | tctcccggacccctgaggtcacatgcgtggtggtggacgtgagccacgaagaccctga |
| | | | | ggtcaagttcaactggtacgtggacggcgtggaggtgcataatgccaagacaaagccg |
| | | | | cgggaggagcagtacaacagcacgtaccgggtggtcagcgtcctcaccgtcctgcacc |
| | | | | aggactggctgaatggcaaggagtacaagtgcaaggtctccaacaaagccctcccagc |
| | | | | ccccatcgagaaaaccatctccaaagccaaagggcagccccgagaaccacaggtgtac |
| | | | | accctgcccccatcccgggatgagctgaccaagaaccaggtcagcctgacctgcctgg |
| | | | | tcaaaggcttctatcccagcgacatcgccgtggagtgggagagcaatgggcagccgga |
| | | | | gaacaactacaagaccacgcctcccgtgctggactccgacggctccttcttcctctac |
| | | | | agcaagctcaccgtggacaagagcaggtggcagcaggggaacgtcttctcatgctccg |
| | | | | tgatgcatgaggctctgcacaaccactacacgcagaagagcctctccctgtctccggg |
| | | | | taaa |
| 34 | | | Human Heavy Chain Constant | ASTKGPSVFPLAPSSKSTSGGTAALGCLVKDYFPEPVTVSWNSGALTSGVHTFPAVLQ |
| | | | Region (IGHG1*01) Protein | SSGLYSLSSVVTVPSSSLGTQTYICNVNHKPSNTKVDKKVEPKSCDKTHTCPPCPAPE |
| | | | Sequence (P01857) | LLGGPSVFLFPPKPKDTLMISRTPEVTCVVVDVSHEDPEVKENWYVDGVEVHNAKTKP |
| | | | | REEQYNSTYRVVSVLTVLHQDWLNGKEYKCKVSNKALPAPIEKTISKAKGQPREPQVY |
| | | | | TLPPSRDELTKNQVSLTCLVKGFYPSDIAVEWESNGQPENNYKTTPPVLDSDGSFFLY |
| | | | | SKLTVDKSRWQQGNVFSCSVMHEALHNHYTQKSLSLSPGK |
| 35 | Human | IGHG1*02 | Human Heavy Chain Constant | gcctccaccaagggcccatcggtcttccccctggcaccctcctccaagagcacctctg |
| | IgG1 | or | Region (IGHG1*02 or | ggggcacagcggccctgggctgcctggtcaaggactacttccccgaaccggtgacggt |
| | constant | IGHG1*05 | IGHG1*05) Nucleotide | gtcgtggaactcaggcgccctgaccagcggcgtgcacaccttcccggctgtcctacag |
| | region | | Sequence | tcctcaggactctactccctcagcagcgtggtgaccgtgccctccagcagcttgggca |
| | | | | cccagacctacatctgcaacgtgaatcacaagcccagcaacaccaaggtggacaagaa |
| | | | | agttgagcccaaatcttgtgacaaaactcacacatgcccaccgtgcccagcacctgaa |
| | | | | ctcctggggggaccgtcagtcttcctcttccccccaaaacccaaggacaccctcatga |
| | | | | tctcccggacccctgaggtcacatgcgtggtggtggacgtgagccacgaagaccctga |
| | | | | ggtcaagttcaactggtacgtggacggcgtggaggtgcataatgccaagacaaagccg |
| | | | | cgggaggagcagtacaacagcacgtaccgtgtggtcagcgtcctcaccgtcctgcacc |
| | | | | aggactggctgaatggcaaggagtacaagtgcaaggtctccaacaaagccctcccagc |
| | | | | ccccatcgagaaaaccatctccaaagccaaagggcagccccgagaaccacaggtgtac |
| | | | | accctgcccccatcccgggatgagctgaccaagaaccaggtcagcctgacctgcctgg |
| | | | | tcaaaggcttctatcccagcgacatcgccgtggagtgggagagcaatgggcagccgga |
| | | | | gaacaactacaagaccacgcctcccgtgctggactccgacggctccttcttcctctac |
| | | | | agcaagctcaccgtggacaagagcaggtggcagcaggggaacgtcttctcatgctccg |
| | | | | tgatgcatgaggctctgcacaaccactacacgcagaagagcctctccctgtctccggg |
| | | | | taaa |
| 36 | | | Human Heavy Chain Constant | ASTKGPSVFPLAPSSKSTSGGTAALGCLV |
| | | | Region (IGHG1*02) Protein | KDYFPEPVTVSWNSGALTSGVHTFPAVLQ |
| | | | Sequence | SSGLYSLSSVVTVPSSSLGTQTYICNVNH |
| | | | | KPSNTKTDKKVEPKSCDKTHTCPPCPAPE |
| | | | | LLGGPSVFLFPPKPKDTLMISRTPEVTCV |
| | | | | VVDVSHEDPEVKFNWYVDGVEVHNAKTKP |
| | | | | REEQYNSTYRVVSVLTVLHQDWLNGKEYK |
| | | | | CKVSNKALPAPIEKTISKAKGQPREPQVY |
| | | | | TLPPSRDELTKNQVSLTCLVKGFYPSDIA |
| | | | | VEWESNGQPENNYKTTPPVLDSDGSFFLY |
| | | | | SKLTVDKSRWQQGNVFSCSVMHEALHNHY |
| | | | | TQKSLSLSPGK |
| 37 | Human | IGHG1*03 | Human Heavy Chain Constant | gcctccaccaagggcccatcggtcttccccctggcaccctcctccaagagcacctctg |
| | IgG1 | | Region (IGHG1*03) | ggggcacagcggccctgggctgcctggtcaaggactacttccccgaaccggtgacggt |
| | constant | | Nucleotide Sequence | gtcgtggaactcaggcgccctgaccagcggcgtgcacaccttcccggctgtcctacag |
| | region | | (Y14737) | tcctcaggactctactccctcagcagcgtggtgaccgtgccctccagcagcttgggca |
| | | | | cccagacctacatctgcaacgtgaatcacaagcccagcaacaccaaggggacaagag |
| | | | | agttgagcccaaatcttgtgacaaaactcacacatgcccaccgtgcccagcacctgaa |
| | | | | ctcctggggggaccgtcagtcttcctcttccccccaaaacccaaggacaccctcatga |
| | | | | tctcccggacccctgaggtcacatgcgtggtggtggacgtgagccacgaagaccctga |
| | | | | ggtcaagttcaactggtacgtggacggcgtggaggtgcataatgccaagacaaagccg |
| | | | | cgggaggagcagtacaacagcacgtaccgtgtggtcagcgtcctcaccgtcctgcacc |
| | | | | aggactggctgaatggcaaggagtacaagtgcaaggtctccaacaaagccctcccagc |
| | | | | ccccatcgagaaaaccatctccaaagccaaagggcagccccgagaaccacaggtgtac |
| | | | | accctgcccccatcccgggaggagatgaccaagaaccaggtcagcctgacctgcctgg |
| | | | | tcaaaggcttctatcccagcgacatcgccgtggagtgggagagcaatgggcagccgga |
| | | | | gaacaactacaagaccacgcctcccgtgctggactccgacggctccttcttcctctat |
| | | | | agcaagctcaccgtggacaagagcaggtggcagcaggggaacgtcttctcatgctccg |
| | | | | tgatgcatgaggctctgcacaaccactacacgcagaagagcctctccctgtccccggg |
| | | | | taaa |
| 38 | | | Human Heavy Chain Constant | ASTKGPSVFPLAPSSKSTSGGTAALGCLV |
| | | | Region (IGHG1*03) Protein | KDYFPEPVTVSWNSGALTSGVHTFPAVLQ |
| | | | Sequence | SSGLYSLSSVVTVPSSSLGTQTYICNVNH |
| | | | | KPSNTKVDKRVEPKSCDKTHTCPPCPAPE |
| | | | | LLGGPSVFLFPPKPKDTLMISRTPEVTCV |
| | | | | VVDVSHEDPEVKFNWYVDGVEVHNAKTKP |
| | | | | REEQYNSTYRVVSVLTVLHQDWLNGKEYK |
| | | | | CKVSNKALPAPIEKTISKAKGQPREPQVY |
| | | | | TLPPSREEMTKNQVSLTCLVKGFYPSDIA |
| | | | | VEWESNGQPENNYKTTPPVLDSDGSFFLY |
| | | | | SKLTVDKSRWQQGNVFSCSVMHEALHNHY |
| | | | | TQKSLSLSPGK |
| 39 | Human | IGHG1*04 | Human Heavy Chain Constant | gcctccaccaagggcccatcggtcttccccctggcaccctcctccaagagcacctctg |
| | IgG1 | | Region (IGHG1*04) | ggggcacagcggccctgggctgcctggtcaaggactacttccccgaaccggtgacggt |
| | constant | | Nucleotide Sequence | gtcgtggaactcaggcgccctgaccagcggcgtgcacaccttcccggctgtcctacag |
| | region | | | tcctcaggactctactccctcagcagcgtggtgaccgtgccctccagcagcttgggca |
| | | | | cccagacctacatctgcaacgtgaatcacaagcccagcaacaccaaggtggacaagaa |
| | | | | agttgagcccaaatcttgtgacaaaactcacacatgcccaccgtgcccagcacctgaa |
| | | | | ctcctggggggaccgtcagtcttcctcttccccccaaaacccaaggacaccctcatga |
| | | | | tctcccggacccctgaggtcacatgcgtggtggtggacgtgagccacgaagaccctga |
| | | | | ggtcaagttcaactggtacgtggacggcgtggaggtgcataatgccaagacaaagccg |
| | | | | cgggaggagcagtacaacagcacgtaccgtgtggtcagcgtcctcaccgtcctgcacc |
| | | | | aggactggctgaatggcaaggagtacaagtgcaaggtctccaacaaagccctcccagc |
| | | | | ccccatcgagaaaaccatctccaaagccaaagggcagccccgagaaccacaggtgtac |
| | | | | accctgcccccatcccgggatgagctgaccaagaaccaggtcagcctgacctgcctgg |
| | | | | tcaaaggcttctatcccagcgacatcgccgtggagtgggagagcaatgggcagccgga |
| | | | | gaacaactacaagaccacgcctcccgtgctggactccgacggctccttcttcctctac |
| | | | | agcaagctcaccgtggacaagagcaggtggcagcaggggaacatcttctcatgctccg |
| | | | | tgatgcatgaggctctgcacaaccactacacgcagaagagcctctccctgtctccggg |
| | | | | taaa |
| 40 | | | Human Heavy Chain Constant | ASTKGPSVFPLAPSSKSTSGGTAALGCLVKDYFPEPVTVSWNSGALTSGVHTFPAVLQ |
| | | | Region (IGHG1*04) Protein | SSGLYSLSSVVTVPSSSLGTQTYICNVNHKPSNTKVDKKVEPKSCDKTHTCPPCPAPE |
| | | | Sequence | LLGGPSVFLFPPKPKDTLMISRTPEVTCVVVDVSHEDPEVKENWYVDGVEVHNAKTKP |
| | | | | REEQYNSTYRVVSVLTVLHQDWLNGKEYKCKVSNKALPAPIEKTISKAKGQPREPQVY |
| | | | | TLPPSRDELTKNQVSLTCLVKGFYPSDIAVEWESNGQPENNYKTTPPVLDSDGSFFLY |
| | | | | SKLTVDKSRWQQGNIFSCSVMHEALHNHYTQKSLSLSPGK |
| 41 | Disabled | Disabled | Disabled Human IGHG1*01 | gcctccaccaagggcccatcggtcttccccctggcaccctcctccaagagcacctctg |
| | Human | human | Heavy Chain Constant | ggggcacagcggccctgggctgcctggtcaaggactacttccccgaaccggtgacggt |
| | IgG1 | IGHG1*01 | Region Nucleotide | gtcgtggaactcaggcgccctgaccagcggcgtgcacaccttcccggctgtcctacag |
| | heavy | | Sequence. | tcctcaggactctactccctcagcagcgtggtgaccgtgccctccagcagcttgggca |
| | | | | cccagacctacatctgcaacgtgaatcacaagcccagcaacaccaaggtggacaagaa |
| | | | | agtggagcccaaatcttgtgacaaaactcacacatgcccaccgtgcccagcacctgaa |
| | | | | ctcgcgggggcaccgtcagtcttcctcttccccccaaaacccaaggacaccctcatga |
| | | | | tctcccggacccctgaggtcacatgcgtggtggtggacgtgagccacgaagaccctga |
| | | | | ggtcaagttcaactggtacgtggacggcgtggaggtgcataatgccaagacaaagccg |
| | | | | cgggaggagcagtacaacagcacgtaccgtgtggtcagcgtcctcaccgtcctgcacc |
| | | | | aggactggctgaatggcaaggagtacaagtgcaaggtctccaacaaagccctcccagc |
| | | | | ccccatcgagaaaaccatctccaaagccaaagggcagccccgagaaccacaggtgtac |
| | | | | accctgcccccatcccgggatgagctgaccaagaaccaggtcagcctgacctgcctgg |
| | | | | tcaaaggcttctatcccagcgacatcgccgtggagtgggagagcaatgggcagccgga |
| | | | | gaacaactacaagaccacgcctcccgtgctggactccgacggctccttcttcctctac |
| | | | | agcaagctcaccgtggacaagagcaggtggcagcaggggaacgtcttctcatgctccg |
| | | | | tgatgcatgaggctctgcacaaccactacacgcagaagagcctctccctgtctccggg |
| | | | | taaa |
| 42 | chain | | Disabled Human IGHG1*01 | ASTKGPSVFPLAPSSKSTSGGTAALGCLVKDYFPEPVTVSWNSGALTSGVHTFPAVLQ |
| | constant | | Heavy Chain Constant | SSGLYSLSSVVTVPSSSLGTQTYICNVNHKPSNTKVDKKVEPKSCDKTHTCPPCPAPE |
| | region | | Region Amino Acid | LAGAPSVFLFPPKPKDTLMISRTPEVTCVVVDVSHEDPEVKENWYVDGVEVHNAKTKP |
| | | | Sequence. Two residues | REEQYNSTYRVVSVLTVLHQDWLNGKEYKCKVSNKALPAPIEKTISKAKGQPREPQVY |
| | | | that differ from the wild- | TLPPSRDELTKNQVSLTCLVKGFYPSDIAVEWESNGQPENNYKTTPPVLDSDGSFFLY |
| | | | type sequence are | SKLTVDKSRWQQGNVFSCSVMHEALHNHYTQKSLSLSPGK |
| | | | identified in bold. | |
| 43 | Human | IGHG2*01 | Human Heavy Chain Constant | gcctccaccaagggcccatcggtcttccccctggcgccctgctccaggagcacctccg |
| | IgG2 | or | Region (IGHG2*01 or | agagcacagccgccctgggctgcctggtcaaggactacttccccgaaccggtgacggt |
| | constant | IGHG2*04 | IGHG2*03 or IGHG2*05) | gtcgtggaactcaggcgctctgaccagcggcgtgcacaccttcccagctgtcctacag |
| | region | or | Nucleotide Sequence | tcctcaggactctactccctcagcagcgtggtgaccgtgccctccagcaacttcggca |
| | | IGHG2*05 | | cccagacctacacctgcaacgtagatcacaagcccagcaacaccaaggtggacaagac |
| | | | | agttgagcgcaaatgttgtgtcgagtgcccaccgtgcccagcaccacctgtggcagga |
| | | | | ccgtcagtcttcctcttccccccaaaacccaaggacaccctcatgatctcccggaccc |
| | | | | ctgaggtcacgtgcgtggtggtggacgtgagccacgaagaccccgaggtccagttcaa |
| | | | | ctggtacgtggacggcgtggaggtgcataatgccaagacaaagccacgggaggagcag |
| | | | | ttcaacagcacgttccgtgtggtcagcgtcctcaccgttgtgcaccaggactggctga |
| | | | | acggcaaggagtacaagtgcaaggtctccaacaaaggcctcccagcccccatcgagaa |
| | | | | aaccatctccaaaaccaaagggcagccccgagaaccacaggtgtacaccctgccccca |
| | | | | tcccgggaggagatgaccaagaaccaggtcagcctgacctgcctggtcaaaggcttct |
| | | | | accccagcgacatcgccgtggagtgggagagcaatgggcagccggagaacaactacaa |
| | | | | gaccacacctcccatgctggactccgacggctccttcttcctctacagcaagctcacc |
| | | | | gtggacaagagcaggtggcagcaggggaacgtcttctcatgctccgtgatgcatgagg |
| | | | | ctctgcacaaccactacacgcagaagagcctctccctgtctccgggtaaa |
| 44 | | | Human Heavy Chain Constant | ASTKGPSVFPLAPCSRSTSESTAALGCLVKDYFPEPVTVSWNSGALTSGVHTFPAVLQ |
| | | | Region (IGHG2*01) Protein | SSGLYSLSSVVTVPSSNFGTQTYTCNVDHKPSNTKVDKTVERKCCVECPPCPAPPVAG |
| | | | Sequence | PSVFLFPPKPKDTLMISRTPEVTCVVVDVSHEDPEVQFNWYVDGVEVHNAKTKPREEQ |
| | | | | FNSTFRVVSVLTVVHQDWLNGKEYKCKVSNKGLPAPIEKTISKTKGQPREPQVYTLPP |
| | | | | SREEMTKNQVSLTCLVKGFYPSDIAVEWESNGQPENNYKTTPPMLDSDGSFFLYSKLT |
| | | | | VDKSRWQQGNVFSCSVMHEALHNHYTQKSLSLSPGK |
| 45 | Human | IGHG2*02 | Human Heavy Chain Constant | GCCTCCACCAAGGGCCCATCGGTCTTCCCCCTGGCGCCCTGCTCCAGGAGCACCTCCG |
| | IqG2 | | Region (IGHG2*02) | AGAGCACAGCGGCCCTGGGCTGCCTGGTCAAGGACTACTTCCCCGAACCGGTGACGGT |
| | constant | | Nucleotide Sequence | GTCGTGGAACTCAGGCGCTCTGACCAGCGGCGTGCACACCTTCCCGGCTGTCCTACAG |
| | region | | | TCCTCAGGACTCTACTCCCTCAGCAGCGTGGTGACCGTGACCTCCAGCAACTTCGGCA |
| | | | | CCCAGACCTACACCTGCAACGTAGATCACAAGCCCAGCAACACCAAGGTGGACAAGAC |
| | | | | AGTTGAGCGCAAATGTTGTGTCGAGTGCCCACCGTGCCCAGCACCACCTGTGGCAGGA |
| | | | | CCGTCAGTCTTCCTCTTCCCCCCAAAACCCAAGGACACCCTCATGATCTCCCGGACCC |
| | | | | CTGAGGTCACGTGCGTGGTGGTGGACGTGAGCCACGAAGACCCCGAGGTCCAGTTCAA |
| | | | | CTGGTACGTGGACGGCATGGAGGTGCATAATGCCAAGACAAAGCCACGGGAGGAGCAG |
| | | | | TTCAACAGCACGTTCCGTGTGGTCAGCGTCCTCACCGTCGTGCACCAGGACTGGCTGA |
| | | | | ACGGCAAGGAGTACAAGTGCAAGGTCTCCAACAAAGGCCTCCCAGCCCCCATCGAGAA |
| | | | | AACCATCTCCAAAACCAAAGGGCAGCCCCGAGAACCACAGGTGTACACCCTGCCCCCA |
| | | | | TCCCGGGAGGAGATGACCAAGAACCAGGTCAGCCTGACCTGCCTGGTCAAAGGCTTCT |
| | | | | ACCCCAGCGACATCGCCGTGGAGTGGGAGAGCAATGGGCAGCCGGAGAACAACTACAA |
| | | | | GACCACACCTCCCATGCTGGACTCCGACGGCTCCTTCTTCCTCTACAGCAAGCTCACC |
| | | | | GTGGACAAGAGCAGGTGGCAGCAGGGGAACGTCTTCTCATGCTCCGTGATGCATGAGG |
| | | | | CTCTGCACAACCACTACACACAGAAGAGCCTCTCCCTGTCTCCGGGTAAA |
| 46 | | | Human Heavy Chain Constant | ASTKGPSVFPLAPCSRSTSESTAALGCLVKDYFPEPVTVSWNSGALTSGVHTFPAVLQ |
| | | | Region (IGHG2*02) Protein | SSGLYSLSSVVTVTSSNFGTQTYTCNVDHKPSNTKVDKTVERKCCVECPPCPAPPVAG |
| | | | Sequence | PSVFLFPPKPKDTLMISRTPEVTCVVVDVSHEDPEVQFNWYVDGMEVHNAKTKPREEQ |
| | | | | FNSTFRVVSVLTVVHQDWLNGKEYKCKVSNKGLPAPIEKTISKTKGQPREPQVYTLPP |
| | | | | SREEMTKNQVSLTCLVKGFYPSDIAVEWESNGQPENNYKTTPPMLDSDGSFFLYSKLT |
| | | | | VDKSRWQQGNVFSCSVMHEALHNHYTQKSLSLSPGK |
| 47 | Human | IGHG2*04 | Human Heavy Chain Constant | gcctccaccaagggcccatcggtcttccccctggcgccctgctccaggagcacctccg |
| | IgG2 | | Region (IGHG2*04) | agagcacagcggccctgggctgcctggtcaaggactacttccccgaaccggtgacggt |
| | constant | | Nucleotide Sequence | gtcgtggaactcaggcgctctgaccagcggcgtgcacaccttcccagctgtcctacag |
| | region | | | tcctcaggactctactccctcagcagcgtggtgaccgtgccctccagcagcttgggca |
| | | | | cccagacctacacctgcaacgtagatcacaagcccagcaacaccaaggtggacaagac |
| | | | | agttgagcgcaaatgttgtgtcgagtgcccaccgtgcccagcaccacctgtggcagga |
| | | | | ccgtcagtcttcctcttccccccaaaacccaaggacaccctcatgatctcccggaccc |
| | | | | ctgaggtcacgtgcgtggtggtggacgtgagccacgaagaccccgaggtccagttcaa |
| | | | | ctggtacgtggacggcgtggaggtgcataatgccaagacaaagccacgggaggagcag |
| | | | | ttcaacagcacgttccgtgtggtcagcgtcctcaccgttgtgcaccaggactggctga |
| | | | | acggcaaggagtacaagtgcaaggtctccaacaaaggcctcccagcccccatcgagaa |
| | | | | aaccatctccaaaaccaaagggcagccccgagaaccacaggtgtacaccctgccccca |
| | | | | tcccgggaggagatgaccaagaaccaggtcagcctgacctgcctggtcaaaggcttct |
| | | | | accccagcgacatcgccgtggagtgggagagcaatgggcagccggagaacaactacaa |
| | | | | gaccacacctcccatgctggactccgacggctccttcttcctctacagcaagctcacc |
| | | | | gtggacaagagcaggtggcagcaggggaacgtcttctcatgctccgtgatgcatgagg |
| | | | | ctctgcacaaccactacacgcagaagagcctctccctgtctccgggtaaa |
| 48 | | | Human Heavy Chain Constant | ASTKGPSVFPLAPCSRSTSESTAALGCLVKDYFPEPVTVSWNSGALTSGVHTFPAVLQ |
| | | | Region (IGHG2*04) Protein | SSGLYSLSSVVTVPSSSLGTQTYTCNVDHKPSNTKVDKTVERKCCVECPPCPAPPVAG |
| | | | Sequence | PSVFLFPPKPKDTLMISRTPEVTCVVVDVSHEDPEVQFNWYVDGVEVHNAKTKPREEQ |
| | | | | FNSTFRVVSVLTVVHQDWLNGKEYKCKVSNKGLPAPIEKTISKTKGQPREPQVYTLPP |
| | | | | SREEMTKNQVSLTCLVKGFYPSDIAVEWESNGQPENNYKTTPPMLDSDGSFFLYSKLT |
| | | | | VDKSRWQQGNVFSCSVMHEALHNHYTQKSLSLSPGK |
| 49 | Human | IGHG2*06 | Human Heavy Chain Constant | GCCTCCACCAAGGGCCCATCGGTCTTCCCCCTGGCGCCCTGCTCCAGGAGCACCTCCG |
| | IgG2 | | Region (IGHG2*06) | AGAGCACAGCGGCCCTGGGCTGCCTGGTCAAGGACTACTTCCCCGAACCGGTGACGGT |
| | constant | | Nucleotide Sequence | GTCGTGGAACTCAGGCGCTCTGACCAGCGGCGTGCACACCTTCCCGGCTGTCCTACAG |
| | region | | | TCCTCAGGACTCTACTCCCTCAGCAGCGTGGTGACCGTGCCCTCCAGCAACTTCGGCA |
| | | | | CCCAGACCTACACCTGCAACGTAGATCACAAGCCCAGCAACACCAAGGTGGACAAGAC |
| | | | | AGTTGAGCGCAAATGTTGTGTCGAGTGCCCACCGTGCCCAGCACCACCTGTGGCAGGA |
| | | | | CCGTCAGTCTTCCTCTTCCCCCCAAAACCCAAGGACACCCTCATGATCTCCCGGACCC |
| | | | | CTGAGGTCACGTGCGTGGTGGTGGACGTGAGCCACGAAGACCCCGAGGTCCAGTTCAA |
| | | | | CTGGTACGTGGACGGCGTGGAGGTGCATAATGCCAAGACAAAGCCACGGGAGGAGCAG |
| | | | | TTCAACAGCACGTTCCGTGTGGTCAGCGTCCTCACCGTCGTGCACCAGGACTGGCTGA |
| | | | | ACGGCAAGGAGTACAAGTGCAAGGTCTCCAACAAAGGCCTCCCAGCCCCCATCGAGAA |
| | | | | AACCATCTCCAAAACCAAAGGGCAGCCCCGAGAACCACAGGTGTACACCCTGCCCCCA |
| | | | | TCCCGGGAGGAGATGACCAAGAACCAGGTCAGCCTGACCTGCCTGGTCAAAGGCTTCT |
| | | | | ACCCCAGCGACATCTCCGTGGAGTGGGAGAGCAATGGGCAGCCGGAGAACAACTACAA |
| | | | | GACCACACCTCCCATGCTGGACTCCGACGGCTCCTTCTTCCTCTACAGCAAGCTCACC |
| | | | | GTGGACAAGAGCAGGTGGCAGCAGGGGAACGTCTTCTCATGCTCCGTGATGCATGAGG |
| | | | | CTCTGCACAACCACTACACACAGAAGAGCCTCTCCCTGTCTCCGGGTAAA |
| 50 | | | Human Heavy Chain Constant | ASTKGPSVFPLAPCSRSTSESTAALGCLVKDYFPEPVTVSWNSGALTSGVHTFPAVLQ |
| | | | Region (IGHG2*06) Protein | SSGLYSLSSVVTVPSSNFGTQTYTCNVDHKPSNTKVDKTVERKCCVECPPCPAPPVAG |
| | | | Sequence | PSVFLFPPKPKDTLMISRTPEVTCVVVDVSHEDPEVQFNWYVDGVEVHNAKTKPREEQ |
| | | | | FNSTFRVVSVLTVVHQDWLNGKEYKCKVSNKGLPAPIEKTISKTKGQPREPQVYTLPP |
| | | | | SREEMTKNQVSLTCLVKGFYPSDISVEWESNGQPENNYKTTPPMLDSDGSFFLYSKLT |
| | | | | VDKSRWQQGNVFSCSVMHEALHNHYTQKSLSLSPGK |
| 51 | Human | IGHG4*01 | Human Heavy Chain Constant | gcttccaccaagggcccatccgtcttccccctggcgccctgctccaggagcacctccg |
| | IgG4 | or | Region (IGHG4*01 or | agagcacagccgccctgggctgcctggtcaaggactacttccccgaaccggtgacggt |
| | | IGHG4*04 | IGHG4*04) Nucleotide | gtcgtggaactcaggcgccctgaccagcggcgtgcacaccttcccggctgtcctacag |
| | | | Sequence | tcctcaggactctactccctcagcagcgtggtgaccgtgccctccagcagcttgggca |
| | | | | cgaagacctacacctgcaacgtagatcacaagcccagcaacaccaaggtggacaagag |
| | | | | agttgagtccaaatatggtcccccatgcccatcatgcccagcacctgagttcctgggg |
| | | | | ggaccatcagtcttcctgttccccccaaaacccaaggacactctcatgatctcccgga |
| | | | | cccctgaggtcacgtgcgtggtggtggacgtgagccaggaagaccccgaggtccagtt |
| | | | | caactggtacgtggatggcgtggaggtgcataatgccaagacaaagccgcgggaggag |
| | | | | cagttcaacagcacgtaccgtgtggtcagcgtcctcaccgtcctgcaccaggactggc |
| | | | | tgaacggcaaggagtacaagtgcaaggtctccaacaaaggcctcccgtcctccatcga |
| | | | | gaaaaccatctccaaagccaaagggcagccccgagagccacaggtgtacaccctgccc |
| | | | | ccatcccaggaggagatgaccaagaaccaggtcagcctgacctgcctggtcaaaggct |
| | | | | tctaccccagcgacatcgccgtggagtgggagagcaatgggcagccggagaacaacta |
| | | | | caagaccacgcctcccgtgctggactccgacggctccttcttcctctacagcaggcta |
| | | | | accgtggacaagagcaggtggcaggaggggaatgtcttctcatgctccgtgatgcatg |
| | | | | aggctctgcacaaccactacacacagaagagcctctccctgtctctgggtaaa |
| 52 | constant | | Human Heavy Chain Constant | ASTKGPSVFPLAPCSRSTSESTAALGCLVKDYFPEPVTVSWNSGALTSGVHTFPAVLQ |
| | region | | Region (IGHG4*01) Protein | SSGLYSLSSVVTVPSSSLGTKTYTCNVDHKPSNTKVDKRVESKYGPPCPSCPAPEFLG |
| | | | Sequence (P01861) | GPSVFLFPPKPKDTLMISRTPEVTCVVVDVSQEDPEVQFNWYVDGVEVHNAKTKPREE |
| | | | | QFNSTYRVVSVLTVLHQDWLNGKEYKCKVSNKGLPSSIEKTISKAKGQPREPQVYTLP |
| | | | | PSQEEMTKNQVSLTCLVKGFYPSDIAVEWESNGQPENNYKTTPPVLDSDGSFFLYSRL |
| | | | | TVDKSRWQEGNVFSCSVMHEALHNHYTQKSLSLSLGK |
| 53 | Human | IGHG4*02 | Human Heavy Chain Constant | gcttccaccaagggcccatccgtcttccccctggcgccctgctccaggagcacctccg |
| | IgG4 | | Region (IGHG4*02) | agagcacagccgccctgggctgcctggtcaaggactacttccccgaaccggtgacggt |
| | | | Nucleotide Sequence | gtcgtggaactcaggcgccctgaccagcggcgtgcacaccttcccggctgtcctacag |
| | | | | tcctcaggactctactccctcagcagcgtggtgaccgtgccctccagcagcttgggca |
| | | | | cgaagacctacacctgcaacgtagatcacaagcccagcaacaccaaggtggacaagag |
| | | | | agttgagtccaaatatggtcccccgtgcccatcatgcccagcacctgagttcctgggg |
| | | | | ggaccatcagtcttcctgttccccccaaaacccaaggacactctcatgatctcccgga |
| | | | | cccctgaggtcacgtgcgtggtggtggacgtgagccaggaagaccccgaggtccagtt |
| | | | | caactggtacgtggatggcgtggaggtgcataatgccaagacaaagccgcgggaggag |
| | | | | cagttcaacagcacgtaccgtgtggtcagcgtcctcaccgtcgtgcaccaggactggc |
| | | | | tgaacggcaaggagtacaagtgcaaggtctccaacaaaggcctcccgtcctccatcga |
| | | | | gaaaaccatctccaaagccaaagggcagccccgagagccacaggtgtacaccctgccc |
| | | | | ccatcccaggaggagatgaccaagaaccaggtcagcctgacctgcctggtcaaaggct |
| | | | | tctaccccagcgacatcgccgtggagtgggagagcaatgggcagccggagaacaacta |
| | | | | caagaccacgcctcccgtgctggactccgacggctccttcttcctctacagcaggcta |
| | | | | accgtggacaagagcaggtggcaggaggggaatgtcttctcatgctccgtgatgcatg |
| | | | | aggctctgcacaaccactacacgcagaagagcctctccctgtctctgggtaaa |
| 54 | constant | | Human Heavy Chain Constant | ASTKGPSVFPLAPCSRSTSESTAALGCLVKDYFPEPVTVSWNSGALTSGVHTFPAVLQ |
| | region | | Region (IGHG4*02) Protein | SSGLYSLSSVVTVPSSSLGTKTYTCNVDHKPSNTKVDKRVESKYGPPCPSCPAPEFLG |
| | | | Sequence | GPSVFLFPPKPKDTLMISRTPEVTCVVVDVSQEDPEVQFNWYVDGVEVHNAKTKPREE |
| | | | | QFNSTYRVVSVLTVVHQDWLNGKEYKCKVSNKGLPSSIEKTISKAKGQPREPQVYTLP |
| | | | | PSQEEMTKNQVSLTCLVKGFYPSDIAVEWESNGQPENNYKTTPPVLDSDGSFFLYSRL |
| | | | | TVDKSRWQEGNVFSCSVMHEALHNHYTQKSLSLSLGK |
| 55 | Human | IGHG4*03 | Human Heavy Chain Constant | gcttccaccaagggcccatccgtcttccccctggcgccctgctccaggagcacctccg |
| | IgG4 | | Region (IGHG4*03) | agagcacagccgccctgggctgcctggtcaaggactacttccccgaaccggtgacggt |
| | constant | | Nucleotide Sequence | gtcgtggaactcaggcgccctgaccagcggcgtgcacaccttcccggctgtcctacag |
| | region | | | tcctcaggactctactccctcagcagcgtggtgaccgtgccctccagcagcttgggca |
| | | | | cgaagacctacacctgcaacgtagatcacaagcccagcaacaccaaggtggacaagag |
| | | | | agttgagtccaaatatggtcccccatgcccatcatgcccagcacctgagttcctgggg |
| | | | | ggaccatcagtcttcctgttccccccaaaacccaaggacactctcatgatctcccgga |
| | | | | cccctgaggtcacgtgcgtggtggtggacgtgagccaggaagaccccgaggtccagtt |
| | | | | caactggtacgtggatggcgtggaggtgcataatgccaagacaaagccgcgggaggag |
| | | | | cagttcaacagcacgtaccgtgtggtcagcgtcctcaccgtcctgcaccaggactggc |
| | | | | tgaacggcaaggagtacaagtgcaaggtctccaacaaaggcctcccgtcctccatcga |
| | | | | gaaaaccatctccaaagccaaagggcagccccgagagccacaggtgtacaccctgccc |
| | | | | ccatcccaggaggagatgaccaagaaccaggtcagcctgacctgcctggtcaaaggct |
| | | | | tctaccccagcgacatcgccgtggagtgggagagcaatgggcagccggagaacaacta |
| | | | | caagaccacgcctcccgtgctggactccgacggctccttcttcctctacagcaagctc |
| | | | | accgtggacaagagcaggtggcaggaggggaacgtcttctcatgctccgtgatgcatg |
| | | | | aggctctgcacaaccactacacgcagaagagcctctccctgtctctgggtaaa |
| 56 | | | Human Heavy Chain Constant | ASTKGPSVFPLAPCSRSTSESTAALGCLVKDYFPEPVTVSWNSGALTSGVHTFPAVLQ |
| | | | Region (IGHG4*03) Protein | SSGLYSLSSVVTVPSSSLGTKTYTCNVDHKPSNTKVDKRVESKYGPPCPSCPAPEFLG |
| | | | Sequence | GPSVFLFPPKPKDTLMISRTPEVTCVVVDVSQEDPEVQFNWYVDGVEVHNAKTKPREE |
| | | | | QFNSTYRVVSVLTVLHQDWLNGKEYKCKVSNKGLPSSIEKTISKAKGQPREPQVYTLP |
| | | | | PSQEEMTKNQVSLTCLVKGFYPSDIAVEWESNGQPENNYKTTPPVLDSDGSFFLYSKL |
| | | | | TVDKSRWQEGNVFSCSVMHEALHNHYTQKSLSLSLGK |
| 57 | Human | IGHG4- | Human Heavy Chain Constant | gcctccaccaagggcccatccgtcttccccctggcgccctgctccaggagcacctccg |
| | IgG4-PE | PE | Region (IGHG4-PE) | agagcacggccgccctgggctgcctggtcaaggactacttccccgaaccagtgacggt |
| | constant | | Nucleotide Sequence | gtcgtggaactcaggcgccctgaccagcggcgtgcacaccttcccggctgtcctacag |
| | region | | Version A | tcctcaggactctactccctcagcagcgtggtgaccgtgccctccagcagcttgggca |
| | | | | cgaagacctacacctgcaacgtagatcacaagcccagcaacaccaaggtggacaagag |
| | | | | agttgagtccaaatatggtcccccatgcccaccatgcccagcgcctgaatttgagggg |
| | | | | ggaccatcagtcttcctgttccccccaaaacccaaggacactctcatgatctcccgga |
| | | | | cccctgaggtcacgtgcgtggtggtggacgtgagccaggaagaccccgaggtccagtt |
| | | | | caactggtacgtggatggcgtggaggtgcataatgccaagacaaagccgcgggaggag |
| | | | | cagttcaacagcacgtaccgtgtggtcagcgtcctcaccgtcctgcaccaggactggc |
| | | | | tgaacggcaaggagtacaagtgcaaggtctccaacaaaggcctcccgtcatcgatcga |
| | | | | gaaaaccatctccaaagccaaagggcagccccgagagccacaggtgtacaccctgccc |
| | | | | ccatcccaggaggagatgaccaagaaccaggtcagcctgacctgcctggtcaaaggct |
| | | | | tctaccccagcgacatcgccgtggagtgggagagcaatgggcagccggagaacaacta |
| | | | | caagaccacgcctcccgtgctggactccgacggatccttcttcctctacagcaggcta |
| | | | | accgtggacaagagcaggtggcaggaggggaatgtcttctcatgctccgtgatgcatg |
| | | | | aggctctgcacaaccactacacacagaagagcctctccctgtctctgggtaaa |
| 58 | | | Human Heavy Chain Constant | gcctccaccaagggacctagcgtgttccctctcgccccctgttccaggtccacaagcg |
| | | | Region (IGHG4-PE) | agtccaccgctgccctcggctgtctggtgaaagactactttcccgagcccgtgaccgt |
| | | | Nucleotide Sequence | ctcctggaatagcggagccctgacctccggcgtgcacacatttcccgccgtgctgcag |
| | | | Version B | agcagcggactgtatagcctgagcagcgtggtgaccgtgcccagctccagcctcggca |
| | | | | ccaaaacctacacctgcaacgtggaccacaagccctccaacaccaaggtggacaagcg |
| | | | | ggtggagagcaagtacggccccccttgccctccttgtcctgcccctgagttcgaggga |
| | | | | ggaccctccgtgttcctgtttccccccaaacccaaggacaccctgatgatctcccgga |
| | | | | cacccgaggtgacctgtgtggtcgtggacgtcagccaggaggaccccgaggtgcagtt |
| | | | | caactggtatgtggacggcgtggaggtgcacaatgccaaaaccaagcccagggaggag |
| | | | | cagttcaattccacctacagggtggtgagcgtgctgaccgtcctgcatcaggattggc |
| | | | | tgaacggcaaggagtacaagtgcaaggtgtccaacaagggactgcccagctccatcga |
| | | | | gaagaccatcagcaaggctaagggccagccgagggagccccaggtgtataccctgcct |
| | | | | cctagccaggaagagatgaccaagaaccaagtgtccctgacctgcctggtgaagggat |
| | | | | tctacccctccgacatcgccgtggagtgggagagcaatggccagcccgagaacaacta |
| | | | | caaaacaacccctcccgtgctcgatagcgacggcagcttctttctctacagccggctg |
| | | | | acagtggacaagagcaggtggcaggagggcaacgtgttctcctgttccgtgatgcacg |
| | | | | aggccctgcacaatcactacacccagaagagcctctccctgtccctgggcaag |
| 59 | | | Human Heavy Chain Constant | gccagcaccaagggcccttccgtgttccccctggccccttgcagcaggagcacctccg |
| | | | Region (IGHG4-PE) | aatccacagctgccctgggctgtctggtgaaggactactttcccgagcccgtgaccgt |
| | | | Nucleotide Sequence | gagctggaacagcggcgctctgacatccggcgtccacacctttcctgccgtcctgcag |
| | | | Version C | tcctccggcctctactccctgtcctccgtggtgaccgtgcctagctcctccctcggca |
| | | | | ccaagacctacacctgtaacgtggaccacaaaccctccaacaccaaggtggacaaacg |
| | | | | ggtcgagagcaagtacggccctccctgccctccttgtcctgcccccgagttcgaaggc |
| | | | | ggacccagcgtgttcctgttccctcctaagcccaaggacaccctcatgatcagccgga |
| | | | | cacccgaggtgacctgcgtggtggtggatgtgagccaggaggaccctgaggtccagtt |
| | | | | caactggtatgtggatggcgtggaggtgcacaacgccaagacaaagccccgggaagag |
| | | | | cagttcaactccacctacagggtggtcagcgtgctgaccgtgctgcatcaggactggc |
| | | | | tgaacggcaaggagtacaagtgcaaggtcagcaataagggactgcccagcagcatcga |
| | | | | gaagaccatctccaaggctaaaggccagccccgggaacctcaggtgtacaccctgcct |
| | | | | cccagccaggaggagatgaccaagaaccaggtgagcctgacctgcctggtgaagggat |
| | | | | tctacccttccgacatcgccgtggagtgggagtccaacggccagcccgagaacaatta |
| | | | | taagaccacccctcccgtcctcgacagcgacggatccttctttctgtactccaggctg |
| | | | | accgtggataagtccaggtggcaggaaggcaacgtgttcagctgctccgtgatgcacg |
| | | | | aggccctgcacaatcactacacccagaagtccctgagcctgtccctgggaaag |
| 60 | | | Human Heavy Chain Constant | ASTKGPSVFPLAPCSRSTSESTAALGCLVKDYFPEPVTVSWNSGALTSGVHTFPAVLQ |
| | | | Region (IGHG4-PE) Protein | SSGLYSLSSVVTVPSSSLGTKTYTCNVDHKPSNTKVDKRVESKYGPPCPPCPAPEFEG |
| | | | Sequence | GPSVFLFPPKPKDTLMISRTPEVTCVVVDVSQEDPEVQFNWYVDGVEVHNAKTKPREE |
| | | | | QFNSTYRVVSVLTVLHQDWLNGKEYKCKVSNKGLPSSIEKTISKAKGQPREPQVYTLP |
| | | | | PSQEEMTKNQVSLTCLVKGFYPSDIAVEWESNGQPENNYKTTPPVLDSDGSFFLYSRL |
| | | | | TVDKSRWQEGNVFSCSVMHEALHNHYTQKSLSLSLGK |
| 61 | Inactiva | Inactiva | Inactivated Human Heavy | gcctccaccaagggcccatccgtcttccccctggcgccctgctccaggagcacctccg |
| | ted | ted | Chain Constant Region | agagcacggccgccctgggctgcctggtcaaggactacttccccgaaccagtgacggt |
| | Human | IGHG4 | (IGHG4) Nucleotide | gtcgtggaactcaggcgccctgaccagcggcgtgcacaccttcccggctgtcctacag |
| | IgG4 | | Sequence | tcctcaggactctactccctcagcagcgtggtgaccgtgccctccagcagcttgggca |
| | constant | | | cgaagacctacacctgcaacgtagatcacaagcccagcaacaccaaggtggacaagag |
| | region | | | agttgagtccaaatatggtcccccatgcccaccatgcccagcgcctccagttgcgggg |
| | | | | ggaccatcagtcttcctgttccccccaaaacccaaggacactctcatgatctcccgga |
| | | | | cccctgaggtcacgtgcgtggtggtggacgtgagccaggaagaccccgaggtccagtt |
| | | | | caactggtacgtggatggcgtggaggtgcataatgccaagacaaagccgcgggaggag |
| | | | | cagttcaacagcacgtaccgtgtggtcagcgtcctcaccgtcctgcaccaggactggc |
| | | | | tgaacggcaaggagtacaagtgcaaggtctccaacaaaggcctcccgtcatcgatcga |
| | | | | gaaaaccatctccaaagccaaagggcagccccgagagccacaggtgtacaccctgccc |
| | | | | ccatcccaggaggagatgaccaagaaccaggtcagcctgacctgcctggtcaaaggct |
| | | | | tctaccccagcgacatcgccgtggagtgggagagcaatgggcagccggagaacaacta |
| | | | | caagaccacgcctcccgtgctggactccgacggatccttcttcctctacagcaggcta |
| | | | | accgtggacaagagcaggtggcaggaggggaatgtcttctcatgctccgtgatgcatg |
| | | | | aggctctgcacaaccactacacacagaagagcctctccctgtctctgggtaaa |
| 62 | | | Inactivated Human Heavy | ASTKGPSVFPLAPCSRSTSESTAALGCLVKDYFPEPVTVSWNSGALTSGVHTFPAVLQ |
| | | | Chain Constant Region | SSGLYSLSSVVTVPSSSLGTKTYTCNVDHKPSNTKVDKRVESKYGPPCPPCPAPPVAG |
| | | | (IGHG4) Protein Sequence | GPSVFLFPPKPKDTLMISRTPEVTCVVVDVSQEDPEVQFNWYVDGVEVHNAKTKPREE |
| | | | (inactivating mutations | QFNSTYRVVSVLTVLHQDWLNGKEYKCKVSNKGLPSSIEKTISKAKGQPREPQVYTLP |
| | | | from human IgG4 shown in | PSQEEMTKNQVSLTCLVKGFYPSDIAVEWESNGQPENNYKTTPPVLDSDGSFFLYSRL |
| | | | bold) | TVDKSRWQEGNVFSCSVMHEALHNHYTQKSLSLSLGK |
| 63 | Human Cκ | IGKC*01 | Human Cκ Light Chain | cgtacggtggccgctccctccgtgttcatcttcccaccttccgacgagcagctgaagt |
| | constant | | Constant Region (IGKC*01) | ccggcaccgcttctgtcgtgtgcctgctgaacaacttctacccccgcgaggccaaggt |
| | region | | Nucleotide Sequence | gcagtggaaggtggacaacgccctgcagtccggcaactcccaggaatccgtgaccgag |
| | | | | caggactccaaggacagcacctactccctgtcctccaccctgaccctgtccaaggccg |
| | | | | actacgagaagcacaaggtgtacgcctgcgaagtgacccaccagggcctgtctagccc |
| | | | | cgtgaccaagtctttcaaccggggcgagtgt |
| 64 | | | Cκ Light Chain Constant | RTVAAPSVFIFPPSDEQLKSGTASVVCLLNNFYPREAKVQWKVDNALQSGNSQESVTE |
| | | | Region (IGKC*01) Amino | QDSKDSTYSLSSTLTLSKADYEKHKVYACEVTHQGLSSPVTKSENRGEC |
| | | | Acid Sequence | |
| 65 | Human Cκ | IGKC*02 | Cκ Light Chain Constant | cgaactgtggctgcaccatctgtcttcatcttcccgccatctgatgagcagttgaaat |
| | constant | | Region (IGKC*02) | ctggaactgcctctgttgtgtgcctgctgaataacttctatcccagagaggccaaagt |
| | region | | Nucleotide Sequence | acagtggaaggtggataacgccctccaatcgggtaactcccaggagagtgtcacagag |
| | | | | caggagagcaaggacagcacctacagcctcagcagcaccctgacgctgagcaaagcag |
| | | | | actacgagaaacacaaagtctacgccggcgaagtcacccatcagggcctgagctcgcc |
| | | | | cgtcacaaagagcttcaacaggggagagtgt |
| 66 | | | Cκ Light Chain Constant | RTVAAPSVFIFPPSDEQLKSGTASVVCLLNNFYPREAKVQWKVDNALQSGNSQESVTE |
| | | | Region (IGKC*02) Amino | QESKDSTYSLSSTLTLSKADYEKHKVYAGEVTHQGLSSPVTKSENRGEC |
| | | | Acid Sequence | |
| 67 | Human Cκ | IGKC*03 | Cκ Light Chain Constant | cgaactgtggctgcaccatctgtcttcatcttcccgccatctgatgagcagttgaaat |
| | constant | | Region (IGKC*03) | ctggaactgcctctgttgtgtgcctgctgaataacttctatcccagagaggccaaagt |
| | region | | Nucleotide Sequence | acagcggaaggtggataacgccctccaatcgggtaactcccaggagagtgtcacagag |
| | | | | caggagagcaaggacagcacctacagcctcagcagcaccctgacgctgagcaaagcag |
| | | | | actacgagaaacacaaagtctacgcctgcgaagtcacccatcagggcctgagctcgcc |
| | | | | cgtcacaaagagcttcaacaggggagagtgt |
| 68 | | | Cκ Light Chain Constant | RTVAAPSVFIFPPSDEQLKSGTASVVCLLNNFYPREAKVQRKVDNALQSGNSQESVTE |
| | | | Region (IGKC*03) Amino | QESKDSTYSLSSTLTLSKADYEKHKVYACEVTHQGLSSPVTKSENRGEC |
| | | | Acid Sequence | |
| 69 | Human Cκ | IGKC*04 | Cκ Light Chain Constant | cgaactgtggctgcaccatctgtcttcatcttcccgccatctgatgagcagttgaaat |
| | constant | | Region (IGKC*04) | ctggaactgcctctgttgtgtgcctgctgaataacttctatcccagagaggccaaagt |
| | region | | Nucleotide Sequence | acagtggaaggtggataacgccctccaatcgggtaactcccaggagagtgtcacagag |
| | | | | caggacagcaaggacagcacctacagcctcagcagcaccctgacgctgagcaaagcag |
| | | | | actacgagaaacacaaactctacgcctgcgaagtcacccatcagggcctgagctcgcc |
| | | | | cgtcacaaagagcttcaacaggggagagtgt |
| 70 | | | Cκ Light Chain Constant | RTVAAPSVFIFPPSDEQLKSGTASVVCLLNNFYPREAKVQWKVDNALQSGNSQESVTE |
| | | | Region (IGKC*04) Amino | QDSKDSTYSLSSTLTLSKADYEKHKLYACEVTHQGLSSPVTKSENRGEC |
| | | | Acid Sequence | |
| 71 | Human Cκ | IGKC*05 | Cκ Light Chain Constant | cgaactgtggctgcaccatctgtcttcatcttcccgccatctgatgagcagttgaaat |
| | constant | | Region (IGKC*05) | ctggaactgcctctgttgtgtgcctgctgaataacttctatcccagagaggccaaagt |
| | region | | Nucleotide Sequence | acagtggaaggtggataacgccctccaatcgggtaactcccaggagagtgtcacagag |
| | | | | caggacagcaaggacagcacctacagcctcagcaacaccctgacgctgagcaaagcag |
| | | | | actacgagaaacacaaagtctacgcctgcgaagtcacccatcagggcctgagctcgcc |
| | | | | cgtcacaaagagcttcaacaggggagagtgc |
| 72 | | | Cκ Light Chain Constant | RTVAAPSVFIFPPSDEQLKSGTASVVCLLNNFYPREAKVQWKVDNALQSGNSQESVTE |
| | | | Region (IGKC*05) Amino | QDSKDSTYSLSNTLTLSKADYEKHKVYACEVTHQGLSSPVTKSENRGEC |
| | | | Acid Sequence | |
| 73 | Human Cλ | IGLC1*01 | Cλ Light Chain Constant | cccaaggccaaccccacggtcactctgttcccgccctcctctgaggagctccaagcca |
| | constant | | Region (IGLC1*01) | acaaggccacactagtgtgtctgatcagtgacttctacccgggagctgtgacagtggc |
| | region | | Nucleotide Sequence | ttggaaggcagatggcagccccgtcaaggcgggagtggagacgaccaaaccctccaaa |
| | | | (ENST00000390321.2) | cagagcaacaacaagtacgcggccagcagctacctgagcctgacgcccgagcagtgga |
| | | | | agtcccacagaagctacagctgccaggtcacgcatgaagggagcaccgtggagaagac |
| | | | | agtggcccctacagaatgttca |
| 74 | | | Cλ Light Chain Constant | PKANPTVTLFPPSSEELQANKATLVCLISDFYPGAVTVAWKADGSPVKAGVETTKPSK |
| | | | Region (IGLC1*01) Amino | QSNNKYAASSYLSLTPEQWKSHRSYSCQVTHEGSTVEKTVAPTECS |
| | | | Acid Sequence (A0A075B6K8) | |
| 75 | Human Cλ | IGLC1*02 | Cλ Light Chain Constant | ggtcagcccaaggccaaccccactgtcactctgttcccgccctcctctgaggagctcc |
| | constant | | Region (IGLC1*02) | aagccaacaaggccacactagtgtgtctgatcagtgacttctacccgggagctgtgac |
| | region | | Nucleotide Sequence | agtggcctggaaggcagatggcagccccgtcaaggcgggagtggagaccaccaaaccc |
| | | | Version A | tccaaacagagcaacaacaagtacgcggccagcagctacctgagcctgacgcccgagc |
| | | | | agtggaagtcccacagaagctacagctgccaggtcacgcatgaagggagcaccgtgga |
| | | | | gaagacagtggcccctacagaatgttca |
| 76 | | | Cλ Light Chain Constant | ggtcagcccaaggccaaccccactgtcactctgttcccgccctcctctgaggagctcc |
| | | | Region (IGLC1*02) | aagccaacaaggccacactagtgtgtctgatcagtgacttctacccgggagctgtgac |
| | | | Nucleotide Sequence | agtggcctggaaggcagatggcagccccgtcaaggcgggagtggagaccaccaaaccc |
| | | | Version B | tccaaacagagcaacaacaagtacgcggccagcagctacctgagcctgacgcccgagc |
| | | | | agtggaagtcccacagaagctacagctgccaggtcacgcatgaagggagcaccgtgga |
| | | | | gaagacagtggcccctacagaatgttca |
| 77 | | | Cλ Light Chain Constant | GQPKANPTVTLFPPSSEELQANKATLVCLISDFYPGAVTVAWKADGSPVKAGVETTKP |
| | | | Region (IGLC1*02) Amino | SKQSNNKYAASSYLSLTPEQWKSHRSYSCQVTHEGSTVEKTVAPTECS |
| | | | Acid Sequence | |
| 78 | Human Cλ | IGLC2*01 | Cλ Light Chain Constant | ggccagcctaaggccgctccttctgtgaccctgttccccccatcctccgaggaactgc |
| | constant | | Region (IGLC2*01) | aggctaacaaggccaccctcgtgtgcctgatcagcgacttctaccctggcgccgtgac |
| | region | | Nucleotide Sequence | cgtggcctggaaggctgatagctctcctgtgaaggccggcgtggaaaccaccacccct |
| | | | Version A | tccaagcagtccaacaacaaatacgccgcctcctcctacctgtccctgacccctgagc |
| | | | | agtggaagtcccaccggtcctacagctgccaagtgacccacgagggctccaccgtgga |
| | | | | aaagaccgtggctcctaccgagtgctcc |
| 79 | | | Cλ Light Chain Constant | ggccagcctaaagctgcccccagcgtcaccctgtttcctccctccagcgaggagctcc |
| | | | Region (IGLC2*01) | aggccaacaaggccaccctcgtgtgcctgatctccgacttctatcccggcgctgtgac |
| | | | Nucleotide Sequence | cgtggcttggaaagccgactccagccctgtcaaagccggcgtggagaccaccacaccc |
| | | | Version B | tccaagcagtccaacaacaagtacgccgcctccagctatctctccctgacccctgagc |
| | | | | agtggaagtcccaccggtcctactcctgtcaggtgacccacgagggctccaccgtgga |
| | | | | aaagaccgtcgcccccaccgagtgctcc |
| 80 | | | Cλ Light Chain Constant | GQPKAAPSVTLFPPSSEELQANKATLVCLISDFYPGAVTVAWKADSSPVKAGVETTTP |
| | | | Region (IGLC1*02) Amino | SKQSNNKYAASSYLSLTPEQWKSHRSYSCQVTHEGSTVEKTVAPTECS |
| | | | Acid Sequence | |
| 81 | Human Cλ | IGLC2*02 | Cλ Light Chain Constant | ggtcagcccaaggctgccccctcggtcactctgttcccgccctcctctgaggagcttc |
| | constant | or | Region (IGLC2*02 or | aagccaacaaggccacactggtgtgtctcataagtgacttctacccgggagccgtgac |
| | region | IGLC2*03 | IGLC2*03) Nucleotide | agtggcctggaaggcagatagcagccccgtcaaggcgggagtggagaccaccacaccc |
| | | | Sequence | tccaaacaaagcaacaacaagtacgcggccagcagctatctgagcctgacgcctgagc |
| | | | | agtggaagtcccacagaagctacagctgccaggtcacgcatgaagggagcaccgtgga |
| | | | | gaagacagtggcccctacagaatgttca |
| 82 | | | Cλ Light Chain Constant | GQPKAAPSVTLFPPSSEELQANKATLVCLISDFYPGAVTVAWKADSSPVKAGVETTTP |
| | | | Region (IGLC2*02) Amino | SKQSNNKYAASSYLSLTPEQWKSHRSYSCQVTHEGSTVEKTVAPTECS |
| | | | Acid Sequence | |
| 83 | Human Cλ | IGLC3*01 | Cλ Light Chain Constant | cccaaggctgccccctcggtcactctgttcccaccctcctctgaggagcttcaagcca |
| | constant | | Region (IGLC3*01) | acaaggccacactggtgtgtctcataagtgacttctacccgggagccgtgacagttgc |
| | region | | Nucleotide Sequence | ctggaaggcagatagcagccccgtcaaggcgggggtggagaccaccacaccctccaaa |
| | | | | caaagcaacaacaagtacgcggccagcagctacctgagcctgacgcctgagcagtgga |
| | | | | agtcccacaaaagctacagctgccaggtcacgcatgaagggagcaccgtggagaagac |
| | | | | agttgcccctacggaatgttca |
| 84 | | | Cλ Light Chain Constant | PKAAPSVTLFPPSSEELQANKATLVCLISDFYPGAVTVAWKADSSPVKAGVETTTPSK |
| | | | Region (IGLC3*01) Amino | QSNNKYAASSYLSLTPEQWKSHKSYSCQVTHEGSTVEKTVAPTECS |
| | | | Acid Sequence | |
| 85 | Human Cλ | IGLC3*02 | Cλ Light Chain Constant | ggtcagcccaaggctgccccctcggtcactctgttcccaccctcctctgaggagcttc |
| | constant | | Region (IGLC3*02) | aagccaacaaggccacactggtgtgtctcataagtgacttctacccggggccagtgac |
| | region | | Nucleotide Sequence | agttgcctggaaggcagatagcagccccgtcaaggcgggggtggagaccaccacaccc |
| | | | | tccaaacaaagcaacaacaagtacgcggccagcagctacctgagcctgacgcctgagc |
| | | | | agtggaagtcccacaaaagctacagctgccaggtcacgcatgaagggagcaccgtgga |
| | | | | gaagacagtggcccctacggaatgttca |
| 86 | | | Cλ Light Chain Constant | GQPKAAPSVTLFPPSSEELQANKATLVCLISDFYPGPVTVAWKADSSPVKAGVETTTP |
| | | | Region (IGLC1*02) Amino | SKQSNNKYAASSYLSLTPEQWKSHKSYSCQVTHEGSTVEKTVAPTECS |
| | | | Acid Sequence | |
| 87 | Human Cλ | IGLC3*03 | Cλ Light Chain Constant | ggtcagcccaaggctgccccctcggtcactctgttcccaccctcctctgaggagcttc |
| | constant | | Region (IGLC3*03) | aagccaacaaggccacactggtgtgtctcataagtgacttctacccgggagccgtgac |
| | region | | Nucleotide Sequence | agtggcctggaaggcagatagcagccccgtcaaggcgggagtggagaccaccacaccc |
| | | | | tccaaacaaagcaacaacaagtacgcggccagcagctacctgagcctgacgcctgagc |
| | | | | agtggaagtcccacaaaagctacagctgccaggtcacgcatgaagggagcaccgtgga |
| | | | | gaagacagtggcccctacagaatgttca |
| 88 | | | Cλ Light Chain Constant | GQPKAAPSVTLFPPSSEELQANKATLVCLISDFYPGAVTVAWKADSSPVKAGVETTTP |
| | | | Region (IGLC3*03) Amino | SKQSNNKYAASSYLSLTPEQWKSHKSYSCQVTHEGSTVEKTVAPTECS |
| | | | Acid Sequence | |
| 89 | Human Cλ | IGLC3*04 | Cλ Light Chain Constant | ggtcagcccaaggctgccccctcggtcactctgttcccgccctcctctgaggagcttc |
| | constant | | Region (IGLC3*04) | aagccaacaaggccacactggtgtgtctcataagtgacttctacccgggagccgtgac |
| | region | | Nucleotide Sequence | agtggcctggaaggcagatagcagccccgtcaaggcgggagtggagaccaccacaccc |
| | | | | tccaaacaaagcaacaacaagtacgcggccagcagctacctgagcctgacgcctgagc |
| | | | | agtggaagtcccacagaagctacagctgccaggtcacgcatgaagggagcaccgtgga |
| | | | | gaagacagtggcccctacagaatgttca |
| 90 | | | Cλ Light Chain Constant | GQPKAAPSVTLFPPSSEELQANKATLVCLISDFYPGAVTVAWKADSSPVKAGVETTTP |
| | | | Region (IGLC3*04) Amino | SKQSNNKYAASSYLSLTPEQWKSHRSYSCQVTHEGSTVEKTVAPTECS |
| | | | Acid Sequence | |
| 91 | Human Cλ | IGLC6*01 | Cλ Light Chain Constant | ggtcagcccaaggctgccccatcggtcactctgttcccgccctcctctgaggagcttc |
| | constant | | Region (IGLC6*01) | aagccaacaaggccacactggtgtgcctgatcagtgacttctacccgggagctgtgaa |
| | region | | Nucleotide Sequence | agtggcctggaaggcagatggcagccccgtcaacacgggagtggagaccaccacaccc |
| | | | | tccaaacagagcaacaacaagtacgcggccagcagctacctgagcctgacgcctgagc |
| | | | | agtggaagtcccacagaagctacagctgccaggtcacgcatgaagggagcaccgtgga |
| | | | | gaagacagtggcccctgcagaatgttca |
| 92 | | | Cλ Light Chain Constant | GQPKAAPSVTLFPPSSEELQANKATLVCLISDFYPGAVKVAWKADGSPVNTGVETTTP |
| | | | Region (IGLC6*01) Amino | SKQSNNKYAASSYLSLTPEQWKSHRSYSCQVTHEGSTVEKTVAPAECS |
| | | | Acid Sequence | |
| 93 | Human Cλ | IGLC7*01 | Cλ Light Chain Constant | ggtcagcccaaggctgccccatcggtcactctgttcccaccctcctctgaggagcttc |
| | constant | or | Region (IGLC7*01 or | aagccaacaaggccacactggtgtgtctcgtaagtgacttctacccgggagccgtgac |
| | region | | IGLC7*02) Nucleotide | agtggcctggaaggcagatggcagccccgtcaaggtgggagtggagaccaccaaaccc |
| | | | Sequence | tccaaacaaagcaacaacaagtatgcggccagcagctacctgagcctgacgcccgagc |
| | | | | agtggaagtcccacagaagctacagctgccgggtcacgcatgaagggagcaccgtgga |
| | | | | gaagacagtggcccctgcagaatgctct |
| 94 | | IGLC7*02 | Cλ Light Chain Constant | GQPKAAPSVTLFPPSSEELQANKATLVCLVSDFYPGAVTVAWKADGSPVKVGVETTKP |
| | | | Region (IGLC7*01) Amino | SKQSNNKYAASSYLSLTPEQWKSHRSYSCRVTHEGSTVEKTVAPAECS |
| | | | Acid Sequence | |
| 95 | Human Cλ | IGLC7*03 | Cλ Light Chain Constant | GGTCAGCCCAAGGCTGCCCCCTCGGTCACTCTGTTCCCACCCTCCTCTGAGGAGCTTC |
| | constant | | Region (IGLC7*03) | AAGCCAACAAGGCCACACTGGTGTGTCTCGTAAGTGACTTCAACCCGGGAGCCGTGAC |
| | region | | Nucleotide Sequence | AGTGGCCTGGAAGGCAGATGGCAGCCCCGTCAAGGTGGGAGTGGAGACCACCAAACCC |
| | | | | TCCAAACAAAGCAACAACAAGTATGCGGCCAGCAGCTACCTGAGCCTGACGCCCGAGC |
| | | | | AGTGGAAGTCCCACAGAAGCTACAGCTGCCGGGTCACGCATGAAGGGAGCACCGTGGA |
| | | | | GAAGACAGTGGCCCCTGCAGAATGCTCT |
| 96 | | | Cλ Light Chain Constant | GQPKAAPSVTLFPPSSEELQANKATLVCLVSDENPGAVTVAWKADGSPVKVGVETTKP |
| | | | Region (IGLC7*03) Amino | SKQSNNKYAASSYLSLTPEQWKSHRSYSCRVTHEGSTVEKTVAPAECS |
| | | | Acid Sequence | |

TABLE G
Human germline V, D and J gene segments generate VH domains of antibodies through recombination,
and human germline V and J gene segments generate the VL domains of antibodies through
recombination. Nucleotide and/or amino acid sequences of antibody variable domains can be
compared against human germline gene segments to identify the closest matching gene segments.
Table G1 below shows the germline gene segments identified for the QUEL antibodies.

| | IGH V gene | IGH D gene | IGH J gene | IGL V gene | IGL J gene |
|---|---|---|---|---|---|
| QUEL-0101 | IGHV3-30*18 | IGHD3-10*01 | IGHJ6*02 | IGKV1-27*01 | IGKJ4*01 |
| QUEL-0102 | ″ | ″ | ″ | ″ | ″ |
| QUEL-0103 | ″ | ″ | ″ | ″ | ″ |
| QUEL-0104 | ″ | ″ | ″ | ″ | ″ |
| QUEL-0105 | ″ | ″ | ″ | ″ | ″ |
| QUEL-0201 | IGHV1-3*01 | IGHD5-18*01 | IGHJ6*02 | IGLV1-40*01 | IGLJ3*02 |
| QUEL-0301 | IGHV3-7*01 | IGHD1-7*01 | IGHJ4*02 | IGLV1-44*01 | IGLJ3*02 |
| QUEL-0302 | ″ | IGHD1-20*01 | ″ | IGLV1-47*01 | ″ |
| QUEL-0303 | ″ | IGHD1-7*01 | ″ | IGLV1-44*01 | ″ |
| QUEL-0304 | ″ | IGHD1-7*01 | ″ | IGLV1-44*01 | ″ |

Table G2 below shows variable domains with germline v and j framework regions corresponding to
QUEL-0101, QUEL-0201 and QUEL-0301. Antibodies of the present invention optionally comprise
framework regions with germline residues as shown here.

| | VH_JH germline | VL_JL germline |
|---|---|---|
| QUEL-0101 | QVQLVESGGG VVQPGRSLRL | DIQMTQSPSS LSASVGDRVT |
| | SCAASGFTES SYGMHWVRQA | ITCRASQGIS NYLAWYQQKP |
| | PGKGLEWVAV ISYDGSNKYY | GKVPKLLIYA ASTLQSGVPS |
| | ADSVKGRFTI SRDNSKNTLY | RFSGSGSGTD FTLTISSLQP |
| | LQMNSLRAED TAVYYCAKDT | EDVATYYCQK YNSAPPSFGG |
| | TTTTVWTWGQ GTTVTVSS | GTKVEIK |
| | (SEQ ID NO: 1) | (SEQ ID NO: 7) |
| QUEL-0201 | QVQLVQSGAE VKKPGASVKV | QSVLTQPPSV SGAPGQRVTI |
| | SCKASGYTFT SYAMHWVRQA | SCTGSSSNIG AGYDVHWYQQ |
| | PGQRLEWMGW INAGNGNTKY | LPGTAPKLLI YGNSNRPSGV |
| | SQKFQGRVTI TRDTSASTAY | PDRFSGSKSG TSASLAITGL |
| | MELSSLRSED TAVYYCARDT | QAEDEADYYC QSYDSSLSGS |
| | TTTTVWTWGQ GTTVTVSS | GFGGGTKLTV L |
| | (SEQ ID NO: 12) | (SEQ ID NO: 17) |
| QUEL-0301 | EVQLVESGGG LVQPGGSLRL | QSVLTQPPSA SGTPGQRVTI |
| | SCAASGFTFS SYWMSWVRQA | SCSGSSSNIG SNTVNWYQQL |
| | PGKGLEWVAN IKQDGSEKYY | PGTAPKLLIY SNNQRPSGVP |
| | VDSVKGRFTI SRDNAKNSLY | DRFSGSKSGT SASLAISGLQ |
| | LOMNSLRAED TAVYYCARDT | SEDEADYYCA AWDDSLNGPG |
| | LTWGQGTLVT VSS | FGGGTKLTVL |
| | (SEQ ID NO: 22) | (SEQ ID NO: 27) |

1. An antibody that binds human GFRAL, comprising an antibody heavy chain variable (VH) domain and an antibody light chain variable (VL) domain, wherein:
(i) the VH domain comprises the QUEL-0201 set of heavy chain complementarity determining regions (HCDRs) HCDR1 SEQ ID NO: 13, HCDR2 SEQ ID NO: 14 and HCDR3 SEQ ID NO: 15, and
the VL domain comprises the QUEL-0201 set of light chain complementarity determining regions (LCDRs) LCDR1 SEQ ID NO: 18, LCDR2 SEQ ID NO: 19 and LCDR3 SEQ ID NO: 20,
(ii) the VH domain comprises the QUEL-0301 set of heavy chain complementarity determining regions (HCDRs) HCDR1 SEQ ID NO: 23, HCDR2 SEQ ID NO: 24 and HCDR3 SEQ ID NO: 25, and
the VL domain comprises the QUEL-0301 set of light chain complementarity determining regions (LCDRs) LCDR1 SEQ ID NO: 28, LCDR2 SEQ ID NO: 29 and LCDR3 SEQ ID NO: 30,
(iii) the VH domain comprises the QUEL-0101 set of heavy chain complementarity determining regions (HCDRs) HCDR1 SEQ ID NO: 3, HCDR2 SEQ ID NO: 4 and HCDR3 SEQ ID NO: 5, and
the VL domain comprises the QUEL-0101 set of light chain complementarity determining regions (LCDRs) LCDR1 SEQ ID NO: 8, LCDR2 SEQ ID NO: 9 and LCDR3 SEQ ID NO: 10,
(iv) the VH domain has at least 90% amino acid sequence identity to the QUEL-0201 VH domain SEQ ID NO: 12 and
the VL domain has at least 90% amino acid sequence identity to the QUEL-0201 VL domain SEQ ID NO: 17,
(v) the VH domain has at least 90% amino acid sequence identity to the QUEL-0301 VH domain SEQ ID NO: 22 and
the VL domain has at least 90% amino acid sequence identity to the QUEL-0301 VL domain SEQ ID NO: 27, or
(vi) the VH domain has at least 90% amino acid sequence identity to the QUEL-0101 VH domain SEQ ID NO: 2 and
the VL domain having at least 90% amino acid sequence identity to the QUEL-0101 VL domain SEQ ID NO: 7.

2-5. (canceled)

6. The antibody according to claim 1, which
(i) binds human GFRAL with an affinity of 1 nM or stronger as determined by surface plasmon resonance,
(ii) cross-reacts with mouse GFRAL, having an affinity for mouse GFRAL within 10-fold of its affinity for human GFRAL, and/or
(iii) inhibits GFRAL with a potency of 15 nM or stronger, wherein the potency is determined as IC50 in an in vitro assay of ERK phosphorylation in response to GDF15.

7. The antibody according to claim 6, wherein:
the VH domain comprises the QUEL-0201 set of heavy chain complementarity determining regions (HCDRs) HCDR1 SEQ ID NO: 13, HCDR2 SEQ ID NO: 14 and HCDR3 SEQ ID NO: 15 and the VL domain comprises the QUEL-0201 set of light chain complementarity determining regions (LCDRs) LCDR1 SEQ ID NO: 18, LCDR2 SEQ ID NO: 19 and LCDR3 SEQ ID NO: 20, and
the antibody binds human GFRAL with an affinity of 100 pM or stronger or with an affinity in the range of 50 pM-200 pM, as determined by surface plasmon resonance.

8-11. (canceled)

12. The antibody according to claim 1, comprising
(i) a VH domain having at least 95% amino acid sequence identity to the QUEL-0201 VH domain SEQ ID NO: 12 and
a VL domain having at least 95% amino acid sequence identity to the QUEL-0201 VL domain SEQ ID NO: 17,
(ii) a VH domain having at least 95% amino acid sequence identity to the QUEL-0301 VH domain SEQ ID NO: 22 and
a VL domain having at least 95% amino acid sequence identity to the QUEL-0301 VL domain SEQ ID NO: 27, or
(iii) a VH domain having at least 95% amino acid sequence identity to the QUEL-0101 VH domain SEQ ID NO: 2 and
a VL domain having at least 95% amino acid sequence identity to the QUEL-0101 VL domain SEQ ID NO: 7.

13. The antibody according to claim 1, comprising
(i) a VH domain having at least 98% amino acid sequence identity to the QUEL-0201 VH domain SEQ ID NO: 12 and
a VL domain having at least 98% amino acid sequence identity to the QUEL-0201 VL domain SEQ ID NO: 17.
(ii) a VH domain having at least 98% amino acid sequence identity to the QUEL-0301 VH domain SEQ ID NO: 22 and
a VL domain having at least 98% amino acid sequence identity to the QUEL-0301 VL domain SEQ ID NO: 27, or
(iii) a VH domain having at least 98% amino acid sequence identity to the QUEL-0101 VH domain SEQ ID NO: 2 and
a VL domain having at least 98% amino acid sequence identity to the QUEL-0101 VL domain SEQ ID NO: 7.

14. The antibody according to claim 13, comprising
(i) the QUEL-0201 VH domain SEQ ID NO: 12, optionally with one or two amino acid alterations and
the QUEL-0201 VL domain SEQ ID NO: 17, optionally with one or two amino acid alterations,
(ii) the QUEL-0301 VH domain SEQ ID NO: 22, optionally with one or two amino acid alterations and
the QUEL-0301 VL domain SEQ ID NO: 27, optionally with one or two amino acid alterations, or
(iii) the QUEL-0101 VH domain SEQ ID NO: 2, optionally with one or two amino acid alterations and
the QUEL-0101 VL domain SEQ ID NO: 7, optionally with one or two amino acid alterations.

15. The antibody according to claim 14, comprising
(i) the QUEL-0201 VH domain SEQ ID NO: 12 comprising the QUEL-0201 set of HCDRs HCDR1 SEQ ID NO: 13, HCDR2 SEQ ID NO: 14 and HCDR3 SEQ ID NO: 15, optionally with one or two amino acid alterations in the VH domain framework, and
the QUEL-0201 VL domain SEQ ID NO: 17 comprising the QUEL-0201 set of LCDRs LCDR1 SEQ ID NO: 18, LCDR2 SEQ ID NO: 19 and LCDR3 SEQ ID NO: 20, optionally with one or two amino acid alterations in the VL domain framework,
(ii) the QUEL-0301 VH domain SEQ ID NO: 22 comprising the QUEL-0301 set of HCDRs HCDR1 SEQ ID NO: 23, HCDR2 SEQ ID NO: 24 and HCDR3 SEQ ID NO: 25, optionally with one or two amino acid alterations in the VH domain framework, and
the QUEL-0301 VL domain SEQ ID NO: 27 comprising the QUEL-0301 set of LCDRs LCDR1 SEQ ID NO: 28, LCDR2 SEQ ID NO: 29 and LCDR3 SEQ ID NO: 30, optionally with one or two amino acid alterations in the VL domain framework, or
(iii) the QUEL-0101 VH domain SEQ ID NO: 2 comprising the QUEL-0101 set of HCDRs HCDR1 SEQ ID NO: 3, HCDR2 SEQ ID NO: 4 and HCDR3 SEQ ID NO: 5, optionally with one or two amino acid alterations in the VH domain framework, and
the QUEL-0101 VL domain SEQ ID NO: 7 comprising the QUEL-0101 set of LCDRs LCDR1 SEQ ID NO: 8, LCDR2 SEQ ID NO: 9 and LCDR3 SEQ ID NO: 10, optionally with one or two amino acid alterations in the VL domain framework.

16. The antibody according to claim 15, wherein the one or two amino acid alterations are conservative substitutions.

17. The antibody according to claim 1, comprising
(i) the QUEL-0201 VH domain SEQ ID NO: 12 and the QUEL-0201 VL domain SEQ ID NO: 17,
(ii) the QUEL-0301 VH domain SEQ ID NO: 22 and the QUEL-0301 VL domain SEQ ID NO: 27, or
(iii) the QUEL-0101 VH domain SEQ ID NO: 2 and the QUEL-0101 VL domain SEQ ID NO: 7.

18-70. (canceled)

71. The antibody according to claim 1, comprising a heavy chain Fc region.

72. The antibody according to claim 71, which is a human IgG.

73. The antibody according to claim 72, which is a human IgG4.

74. The antibody according to claim 73 comprising heavy chain constant region SEQ ID NO: 60.

75. A nucleic acid encoding an antibody as defined in claim 1.

76. A host cell in vitro comprising the nucleic acid as defined in claim 75.

77. A composition comprising an antibody according to claim 1, formulated with a pharmaceutically acceptable excipient.

78. The composition according to claim 77, formulated for intravenous or subcutaneous injection.

79. A method of treating a medical condition associated with the GDF15-GFRAL pathway in a patient, comprising administering a composition according to claim 77 to the patient.

80. (canceled)

81. (canceled)

82. The method according to claim 79, wherein the medical condition is hyperemesis gravidarum, anorexia, cachexia, conditioned taste aversion and/or a side effect of chemotherapy treatment for cancer.

83. The method according to claim 82, wherein the patient is also to receive, or has received, treatment with an anti-cancer chemotherapeutic agent.

84. The method according to claim 82, wherein the medical condition is anorexia or cachexia, in a cancer patient.

85. (canceled)

86. The method according to claim 82, wherein the medical condition is a side effect of treatment with an anti-cancer chemotherapeutic agent.

87. The method according to claim 86, wherein the treatment comprises preventing conditioned taste aversion caused by treatment with an anti-cancer chemotherapeutic agent.

88. The method according to claim 82, wherein the treatment comprises reducing body weight loss and extending survival in a cancer patient.

89. A method of reducing glucocorticoid level in a patient, comprising administering an inhibitor of GDF15 signalling to the patient, wherein the inhibitor is an anti-GFRAL antibody as defined in claim 1.

90. (canceled)

91. (canceled)

92. A method according to claim 89, for normalising the blood level of glucocorticoid in a patient who has been determined to have an elevated blood glucocorticoid level.

93. A method according to claim 89, wherein the patient is a cancer patient.

94. A method according to claim 89, wherein the patient is also to receive, or has received, treatment with a cytotoxic and/or antineoplastic agent.

95-97. (canceled)

98. The antibody according to claim 6, wherein:
the VH domain comprises the QUEL-0301 set of heavy chain complementarity determining regions (HCDRs) HCDR1 SEQ ID NO: 23, HCDR2 SEQ ID NO: 24 and HCDR3 SEQ ID NO: 25 and the VL domain comprises the QUEL-0301 set of light chain complementarity determining regions (LCDRs) LCDR1 SEQ ID NO: 28, LCDR2 SEQ ID NO: 29 and LCDR3 SEQ ID NO: 30, and
the antibody binds human GFRAL with an affinity of 1 pM or stronger or with an affinity in the range of 0.5 pM-2 pM as determined by surface plasmon resonance and/or cross-reacts with mouse GFRAL, having an affinity for mouse GFRAL of 10 nM or stronger as determined by surface plasmon resonance.

99. The antibody according to claim 6, wherein:
the VH domain comprises the QUEL-0101 set of heavy chain complementarity determining regions (HCDRs) HCDR1 SEQ ID NO: 3, HCDR2 SEQ ID NO: 4 and HCDR3 SEQ ID NO: 5 and the VL domain comprises the QUEL-0101 set of light chain complementarity determining regions (LCDRs) LCDR1 SEQ ID NO: 8, LCDR2 SEQ ID NO: 9 and LCDR3 SEQ ID NO: 10, and
the antibody binds human GFRAL with an affinity of 200 pM or stronger or with an affinity in the range of 100 pM-400 pM as determined by surface plasmon resonance and/or inhibits GFRAL with a potency of 5 nM or stronger.

100. The antibody according to claim 6, which inhibits GFRAL with a potency of 10 nM or stronger, wherein the potency is determined as IC50 in an in vitro assay of ERK phosphorylation in response to GDF15.

101. The method according to claim 89, wherein glucocorticoid levels are cortisol levels.
